SEQUENCE LISTING (79 sequences)

SEQ ID NO: 1 - DNA, 1978 bp, Vitis vinifera
  source 1..1978: mol_type = unassigned DNA
agctatggcttctttgccttggtcgctcacaaccttcaccgccatcgccaacaccaccaacatttcagccttcccaccttctcccttgtttcaaagggcttctcatgtccccgtagccagaaaccgaagccgcagatttgctcctagtaaggtgtcgtgcaattctgcgaatggtgatccaaactcggattctacctccgacgttcgagaaacttcctcagggaagttagataggaggaatgtgcttcttggcataggagggctgtatggtgctgctggcggtctcggcgccactaagccattagcctttggggctcccatccaggcaccggatatatccaagtgtggtaccgccaccgtgcctgatggtgtaacgcccacaaattgttgcccgccagtcaccacaaagattatagatttccagctaccttcctcaggttcccccatgcgtaccaggccagctgctcacttggtcagcaaagagtacttagccaagtataaaaaggccattgagctgcagaaagctcttcctgatgatgacccgcgtagtttcaagcaacaggctaatgtccattgcacctattgccaaggggcttatgatcaggttgggtataccgacctagaactccaggttcatgcttcatggcttttcctccctttccaccgttactatctctacttcaatgagagaattcttgcaaagttgatcgacgatcccaccatcgctttggcctattgggcttgggataaccctgatggcatgtatatgccgaccatctatgctagttccccatcatcactctacgacgagaagcgcaacgccaagcacctgcctccgactgtgatcgatctcgactacgatggcaccgaacccacaatccctgatgacgaactaaaaaccgacaatctggcaatcatgtacaaacaaattgtgtcgggtgccacgactcctaagcttttccttggttacccataccgcgccggcgatgcgattgaccctggagcgggtacccttgagcacgtcccacataatatagtccacaaatggactggtcttgctgataagcctagtgaggacatgggaaacttctatactgccggcagagaccccatattcttcggtcaccacgccaatgtcgatcggatgtggaatatatggaaaactataggaggtaaaaatagaaaggatttcacggatacggattggcttgacgccacgttcgtcttctacgacgagaacaaacaacttgttaaagtcaaggtctcggactgtgtcgacacttccaagctgagataccaatatcaggatattcctattccatggctaccaaaaaagacgaaggccaaagcgaagacgaccaccaaaagttccaagtcgggagtagcgaaagcggccgaactcccaaagacgacgatcagcagcatcggagacttcccaaaagctcttaactcagtgataagagtagaagttccaaggccaaagaaatcaagaagcaagaaggagaaagaggatgaggaagaggtgttactgataaaaggaatagagctagatagagagaatttcgtgaagtttgatgtgtacatcaacgacgaagattattcagtgagtaggcctaagaatagtgagtttgcaggaagctttgtgaacgtaccacacaagcatatgaaagaaatgaagacgaagaccaatctgaggttcgcgataaatgagctgttagaggacttgggagccgaagatgatgagagtgtgatcgtgactatagtccctcgtgctgggggcgatgatgtcaccattggtggaattgagatcgagtttgtttccgattgatcccatctttcaatgattatccattatatgtatgtatcaggtaagtcacatctttatgtgactaatggaaaatgtgagacttctctgtactctcccgtcaagtcttttattaatttagagctgtttggtttaaaaaaaaaaaaaaaaaaa

SEQ ID NO: 2 - DNA, 1824 bp, Vitis vinifera
  source 1..1824: mol_type = unassigned DNA
  misc_feature 538..1155: function = Tyrosinase Domain
atggcttctttgccttggtcgctcacaaccttcaccgccatcgccaacaccaccaacatttcagccttcccaccttctcccttgtttcaaagggcttctcatgtccccgtagccagaaaccgaagccgcagatttgctcctagtaaggtgtcgtgcaattctgcgaatggtgatccaaactcggattctacctccgacgttcgagaaacttcctcagggaagttagataggaggaatgtgcttcttggcataggagggctgtatggtgctgctggcggtctcggcgccactaagccattagcctttggggctcccatccaggcaccggatatatccaagtgtggtaccgccaccgtgcctgatggtgtaacgcccacaaattgttgcccgccagtcaccacaaagattatagatttccagctaccttcctcaggttcccccatgcgtaccaggccagctgctcacttggtcagcaaagagtacttagccaagtataaaaaggccattgagctgcagaaagctcttcctgatgatgacccgcgtagtttcaagcaacaggctaatgtccattgcacctattgccaaggggcttatgatcaggttgggtataccgacctagaactccaggttcatgcttcatggcttttcctccctttccaccgttactatctctacttcaatgagagaattcttgcaaagttgatcgacgatcccaccatcgctttggcctattgggcttgggataaccctgatggcatgtatatgccgaccatctatgctagttccccatcatcactctacgacgagaagcgcaacgccaagcacctgcctccgactgtgatcgatctcgactacgatggcaccgaacccacaatccctgatgacgaactaaaaaccgacaatctggcaatcatgtacaaacaaattgtgtcgggtgccacgactcctaagcttttccttggttacccataccgcgccggcgatgcgattgaccctggagcgggtacccttgagcacgtcccacataatatagtccacaaatggactggtcttgctgataagcctagtgaggacatgggaaacttctatactgccggcagagaccccatattcttcggtcaccacgccaatgtcgatcggatgtggaatatatggaaaactataggaggtaaaaatagaaaggatttcacggatacggattggcttgacgccacgttcgtcttctacgacgagaacaaacaacttgttaaagtcaaggtctcggactgtgtcgacacttccaagctgagataccaatatcaggatattcctattccatggctaccaaaaaagacgaaggccaaagcgaagacgaccaccaaaagttccaagtcgggagtagcgaaagcggccgaactcccaaagacgacgatcagcagcatcggagacttcccaaaagctcttaactcagtgataagagtagaagttccaaggccaaagaaatcaagaagcaagaaggagaaagaggatgaggaagaggtgttactgataaaaggaatagagctagatagagagaatttcgtgaagtttgatgtgtacatcaacgacgaagattattcagtgagtaggcctaagaatagtgagtttgcaggaagctttgtgaacgtaccacacaagcatatgaaagaaatgaagacgaagaccaatctgaggttcgcgataaatgagctgttagaggacttgggagccgaagatgatgagagtgtgatcgtgactatagtccctcgtgctgggggcgatgatgtcaccattggtggaattgagatcgagtttgtttccgattga

SEQ ID NO: 3 - protein, 607 aa, Vitis vinifera
  REGION 180..385: note = Tyrosinase Domain
MASLPWSLTTFTAIANTTNISAFPPSPLFQRASHVPVARNRSRRFAPSKVSCNSANGDPNSDSTSDVRETSSGKLDRRNVLLGIGGLYGAAGGLGATKPLAFGAPIQAPDISKCGTATVPDGVTPTNCCPPVTTKIIDFQLPSSGSPMRTRPAAHLVSKEYLAKYKKAIELQKALPDDDPRSFKQQANVHCTYCQGAYDQVGYTDLELQVHASWLFLPFHRYYLYFNERILAKLIDDPTIALAYWAWDNPDGMYMPTIYASSPSSLYDEKRNAKHLPPTVIDLDYDGTEPTIPDDELKTDNLAIMYKQIVSGATTPKLFLGYPYRAGDAIDPGAGTLEHVPHNIVHKWTGLADKPSEDMGNFYTAGRDPIFFGHHANVDRMWNIWKTIGGKNRKDFTDTDWLDATFVFYDENKQLVKVKVSDCVDTSKLRYQYQDIPIPWLPKKTKAKAKTTTKSSKSGVAKAAELPKTTISSIGDFPKALNSVIRVEVPRPKKSRSKKEKEDEEEVLLIKGIELDRENFVKFDVYINDEDYSVSRPKNSEFAGSFVNVPHKHMKEMKTKTNLRFAINELLEDLGAEDDESVIVTIVPRAGGDDVTIGGIEIEFVSD

SEQ ID NO: 4 - DNA, 2121 bp, Vitis vinifera
  source 1..2121: mol_type = unassigned DNA
gtcctaggcctttttcatatccatatcgcctgatcagcaccatggcttctctttctcctccggccaccaccaccactaccaacatctcctccctgcaaccttctcctttgtttcacaaagcttctcaagtctccctagttggaaagcgaaatcctagatttgtgcgtagagtagtatcgtgtaacgctacaaatggtgatgaaaggtcaacctctagctccaaaagtggggaatctccactaggaaagcttgataggaggaatgtgcttcttggcttgggagggctttatggtgtctccggtcttggtgccgctgatccattcgcctttgccgccccgatatctccaccggatctaagcgagtgtggggctgcagacttgcctgctggtgcgcagcccaccaattgttgcccgccagtgagcagcaagatcatagatttcaagctgcctcctaagaattccccatttcgcattaggccggcggctcacttggccagcaaagagtacatagccaagtataaaaaggccattgagctcatgaaagctcttcctgatgatgatccgcggagcttcatgcaacaggccgatgttcattgcgcctactgcaatggggcctaccaccaagttgggtttcccgatttagacctccaagtccacaattcatggctcttcctcccataccaccgttactatctttacttctacgagaaaatcctgggaaagttgattaatgatcccaccttcgctttgccgttttggaactgggatgctcctgctggcatgcaaatgcctgccatgtttgctgaccctaactcaccactctacgacaagcttcgcgacgcccaaacatcagccgccaaagctgatcgatctcgactacacctcaccgacagcaatgataccaatgagcaacaaatttccagcaatctctccattatgtaccggcaaattgtatcgagtgccaagactactagtcttttcatgggagccgcttatcgtgccggcgacgaacctgaccccggccagggtacccttgagaacatcccacatggtccagtccacatatggtgtggtgaccgcacccagccgaatattgaggacatgggcaacttctactctgcggccagagacccaatcttcttctctcaccattctaatgtggaccgcatgtggacggtgtggaaagggttaggaggcaagagaaaggacttcaccgacccagattggctagacgctgggttcgtcttctacgacgagaatgcccaacttgttcgcgtgaaggtccgcgactgtctcgaccctaccaaactcaggtatgcctatcaggaggttgatattccatggctgaaatcccgtccaaagccccgtcaaatattgaagaaggtcgtcaataaattgaagcacaagtctggagtagcgaatgcagccgaaattccaagtagcagcagcatcgtcttccccataaaattggactcaacgttgagagttgatgttccaaggccaaggaaatcaaggagtaagaaggagaaggaggatgaagaagaagtgctggtgatcaaaggaatagagctagaaagggataagttcgtgaagtttgacatattcatcaacgacgaagacgatccagtcagcaggcctgacaacacggagtttgccggaagctttgtgaacgtgccacaccaacactcccatgggaaaaagaagaacacgatcctcaggatagggattagtgaattgttggaggatttggaagccgaagatgatgacagtgtggtggtgactttggtccctcgttatggggccgatgttatcaccattggcggaattgaaattgagcttgcctcttgatcacctcactactcttcatcagaataatggcgccctgatttccatccaggattgcaatgatttccagctctttttgtgtgtgcatcaaatcgaatttcgtcgacacttcctccctttatgtgattggaatgtctgtaataccccatttttggatttgaactatggatttctttgatccactgggacatgatgtaatggggaatgaataaattcaacgttatgtatggaacattagtatgcttaattcgatagccatgattctgcactttgtgta

SEQ ID NO: 5 - DNA, 1806 bp, Vitis vinifera
  source 1..1806: mol_type = unassigned DNA
  misc_feature 520..1140: function = Tyrosinase Domain
atggcttctctttctcctccggccaccaccaccactaccaacatctcctccctgcaaccttctcctttgtttcacaaagcttctcaagtctccctagttggaaagcgaaatcctagatttgtgcgtagagtagtatcgtgtaacgctacaaatggtgatgaaaggtcaacctctagctccaaaagtggggaatctccactaggaaagcttgataggaggaatgtgcttcttggcttgggagggctttatggtgtctccggtcttggtgccgctgatccattcgcctttgccgccccgatatctccaccggatctaagcgagtgtggggctgcagacttgcctgctggtgcgcagcccaccaattgttgcccgccagtgagcagcaagatcatagatttcaagctgcctcctaagaattccccatttcgcattaggccggcggctcacttggccagcaaagagtacatagccaagtataaaaaggccattgagctcatgaaagctcttcctgatgatgatccgcggagcttcatgcaacaggccgatgttcattgcgcctactgcaatggggcctaccaccaagttgggtttcccgatttagacctccaagtccacaattcatggctcttcctcccataccaccgttactatctttacttctacgagaaaatcctgggaaagttgattaatgatcccaccttcgctttgccgttttggaactgggatgctcctgctggcatgcaaatgcctgccatgtttgctgaccctaactcaccactctacgacaagcttcgcgacgcccaaacatcagccgccaaagctgatcgatctcgactacacctcaccgacagcaatgataccaatgagcaacaaatttccagcaatctctccattatgtaccggcaaattgtatcgagtgccaagactactagtcttttcatgggagccgcttatcgtgccggcgacgaacctgaccccggccagggtacccttgagaacatcccacatggtccagtccacatatggtgtggtgaccgcacccagccgaatattgaggacatgggcaacttctactctgcggccagagacccaatcttcttctctcaccattctaatgtggaccgcatgtggacggtgtggaaagggttaggaggcaagagaaaggacttcaccgacccagattggctagacgctgggttcgtcttctacgacgagaatgcccaacttgttcgcgtgaaggtccgcgactgtctcgaccctaccaaactcaggtatgcctatcaggaggttgatattccatggctgaaatcccgtccaaagccccgtcaaatattgaagaaggtcgtcaataaattgaagcacaagtctggagtagcgaatgcagccgaaattccaagtagcagcagcatcgtcttccccataaaattggactcaacgttgagagttgatgttccaaggccaaggaaatcaaggagtaagaaggagaaggaggatgaagaagaagtgctggtgatcaaaggaatagagctagaaagggataagttcgtgaagtttgacatattcatcaacgacgaagacgatccagtcagcaggcctgacaacacggagtttgccggaagctttgtgaacgtgccacaccaacactcccatgggaaaaagaagaacacgatcctcaggatagggattagtgaattgttggaggatttggaagccgaagatgatgacagtgtggtggtgactttggtccctcgttatggggccgatgttatcaccattggcggaattgaaattgagcttgcctcttga

SEQ ID NO: 6 - protein, 601 aa, Vitis vinifera
  REGION 174..380: note = Tyrosinase Domain
MASLSPPATTTTTNISSLQPSPLFHKASQVSLVGKRNPRFVRRVVSCNATNGDERSTSSSKSGESPLGKLDRRNVLLGLGGLYGVSGLGAADPFAFAAPISPPDLSECGAADLPAGAQPTNCCPPVSSKIIDFKLPPKNSPFRIRPAAHLASKEYIAKYKKAIELMKALPDDDPRSFMQQADVHCAYCNGAYHQVGFPDLDLQVHNSWLFLPYHRYYLYFYEKILGKLINDPTFALPFWNWDAPAGMQMPAMFADPNSPLYDKLRDAQTSAAKADRSRLHLTDSNDTNEQQISSNLSIMYRQIVSSAKTTSLFMGAAYRAGDEPDPGQGTLENIPHGPVHIWCGDRTQPNIEDMGNFYSAARDPIFFSHHSNVDRMWTVWKGLGGKRKDFTDPDWLDAGFVFYDENAQLVRVKVRDCLDPTKLRYAYQEVDIPWLKSRPKPRQILKKVVNKLKHKSGVANAAEIPSSSSIVFPIKLDSTLRVDVPRPRKSRSKKEKEDEEEVLVIKGIELERDKFVKFDIFINDEDDPVSRPDNTEFAGSFVNVPHQHSHGKKKNTILRIGISELLEDLEAEDDDSVVVTLVPRYGADVITIGGIEIELAS

SEQ ID NO: 7 - DNA, 1984 bp, Vitis vinifera
  source 1..1984: mol_type = unassigned DNA
tcatcacttctcctctaaagctatggcttctttgccttggtcgctcacaacctccaccgccatcgccaacaccaccaacatttcagccttgccaccttctcccttgtttcacagggtttctcatgtccccatagccagaaaccgaagccgcagatttgctcctagtaaggtgtcgtgcaatgctgcgaatggtgatccaaactcggattctacctccgacattcgagaaacttccccagggaagttagataggaggaatgtgcttcttggcataggagggctgtatggtgctgctgccggtctcggcgccactaagccattagcctttggggctcccatccaggcaccggatatatccaagtgtggtaccgccaccgtgcctgatggtgtaacgcccacaattgttgcccgccagtcaccacaaagattatagatttccagctaccttcctcaggttcccccatgcgtaccaggccagctgctcacttggtcagcaaagagtacttagccaagtataaaaaggccattgagctgcagaaagctcttcctgatgatgatccgcgtagtttcaagcaacaggctaatgtccattgcacctattgccaaggggcttatgatcaggttgggtataccgacctagaactccaggttcatgcttcatggcttttcctccctttccaccgttactatctctacttcaatgagagaattcttgcaaaattgatcgacgatcccaccttcgctttgccctattgggcttgggataaccctgatggcatgtatatgccggccatctatgctagttccccatcatcactttacgacgagaagcgcaacgccaagcacctgcctccgactgtgatcgatctcgactacgatggcaccgaacccacaatccctgatgacgaactaaaagccgacaatctggcaatcatgtacaaacaaattgtgtcgggtgccacgactcctaagcttttctttggttacccataccgcgccggcgatgcgattgaccctggagcgggtacccttgagcttgtcccacataatatagtccacaaatggactggtcttgctgataagcctagtgaggacatgggaaacttctatactgccggcagagaccccatattcttcggtcacacgccaatgtcgatcgaatgtggaatatatggaaaactataggaaggaaaaataaaaaggatttcacgggatccggatggtctggacgccactttggtcttttacgacgggaacaaacaattggtagaagtgaaggtctcggagtggttcgacacttccaagctgagataccaatatcaggatattcttcttccatggctaccaaaaaatacgaaggccaaagcgaagacgaccaccaaaagttccaagtcgggagtagcgaaagcggccgaactcccaaagacgacgatcagcagcatcggagacttcccaaaagctcttaactcagtgataagagtagaagttccaaggccaaagaaatcaagaagcaagaaggagaaagaggacgaggaagaggtgttactgataaaaggaatagagctagatagagagaatttcgtgaagtttgatgtgtacatcaacgacgaagattattcagtgagtaggcctaagaatagtgagtttgcaggaagctttgtgaacgtaccacacaagcatatgaaagaaatgaagacgaagaccaatctgaggttggcgataaatgagctgttagaggacttgggagccgaagatgatgagagtgtgatcgtgactatagtccctcgtgctgggggcgatgatgtcaccattggtggaattgagatcgagtttgtttccgattgatcccacctttcaatgattatccattatatgtatgtatcaggtaagtcacatctttatgtgactaatggaaaatgtgagacttctctgtactctcccgtcaagtcttttattaatttagagctgtttggtttaaatttaa

SEQ ID NO: 8 - DNA, 723 bp, Vitis vinifera
  source 1..723: mol_type = unassigned DNA
  misc_feature 97..684: function = Tyrosinase Domain
atgcgtaccaggccagctgctcacttggtcagcaaagagtacttagccaagtataaaaaggccattgagctgcagaaagctcttcctgatgatgatccgcgtagtttcaagcaacaggctaatgtccattgcacctattgccaaggggcttatgatcaggttgggtataccgacctagaactccaggttcatgcttcatggcttttcctccctttccaccgttactatctctacttcaatgagagaattcttgcaaaattgatcgacgatcccaccttcgctttgccctattgggcttgggataaccctgatggcatgtatatgccggccatctatgctagttccccatcatcactttacgacgagaagcgcaacgccaagcacctgcctccgactgtgatcgatctcgactacgatggcaccgaacccacaatccctgatgacgaactaaaagccgacaatctggcaatcatgtacaaacaaattgtgtcgggtgccacgactcctaagcttttctttggttacccataccgcgccggcgatgcgattgaccctggagcgggtacccttgagcttgtcccacataatatagtccacaaatggactggtcttgctgataagcctagtgaggacatgggaaacttctatactgccggcagagaccccatattcttcggtcacacgccaatgtcgatcgaatgtggaatatatggaaaactatag

SEQ ID NO: 9 - protein, 240 aa, Vitis vinifera
  REGION 33..228: note = Tyrosinase Domain
MRTRPAAHLVSKEYLAKYKKAIELQKALPDDDPRSFKQQANVHCTYCQGAYDQVGYTDLELQVHASWLFLPFHRYYLYFNERILAKLIDDPTFALPYWAWDNPDGMYMPAIYASSPSSLYDEKRNAKHLPPTVIDLDYDGTEPTIPDDELKADNLAIMYKQIVSGATTPKLFFGYPYRAGDAIDPGAGTLELVPHNIVHKWTGLADKPSEDMGNFYTAGRDPIFFGHTPMSIECGIYGKL

SEQ ID NO: 10 - DNA, 1878 bp, Vitis vinifera
  source 1..1878: mol_type = unassigned DNA
gaccgaggccttagcgcgttatcactcatcacttctcctctaaagctatggcttctttgccttggtcgctcacaacctccaccgccatcgccaacaccaccaacatttcagccttgccaccttctcccttgtttcacagggtttctcatgtccccatagccagaaaccgaagccgcagatttgctcctagtaaggtgtcgtgcaatgctgcgaatggtgatccaaactcggattctacctccgacattcgagaaacttccccagggaagttagataggaggaatgtgcttcttggcataggagggctgtatggtgctgctgccggtctcggcgccactaagccattagcctttggggctcccatccaggcaccggatatacgcaagtgtggtaccgccaccgtgcctaatggtgtaattcccacaaattgttgcccgccagtcaccacaaagcctatacctttccagctaccttcctcagattcccccatgcgtaccaggccagctgctcacttggtcagcaaagagtacttagccaagtataaaaaggccattgagctgcagaaagctcttcctgatgatgatccgcgtagtttcaagcaacaggccaatgtccattgcgcctattgccaaggggcttatgatcaggttgggtataccgacctagaactccaggttcatgcttcatgggtttctcatgtccccatagccagaaaccgaagccgcagatttgctcctagtaaggtgtcgtgcaatgctgcgaatggtgatccaaactcggattctacctccgacattcgagaaacttccccagggaagttagataggaggaatgtgcttcttggcataggagggctgtatggtgctgctgccggtctcggcgccactaagccattagcctttggggctcccatccaggcaccggatatatccaagtgtggtaccgccaccgtgcctgatggtgtaacgcccacaattgttgcccgccagccagctgctcacttggtcagcaaagagtacttagccaagtataaaaaggccattgagctgcagaaagctcttcctgatgatgatccgcgtagtttcaagcaacaggctaatgtccattgcacctattgccaaggggcttatgatcaggttgggtataccgacctagaactccagaaagtcaaggtctcggactgtgtcgacacttccaagctgagataccaatatcaggatattncaatcccatggctaccaaaaaatacgaaggccaaagcgaagacgaccaccaaaagttccaagtcgggagtagcgaaagcggccgaactcccaaagacgacgatcagcagcatcggagacttcccaaaagctcttaactcagtgataagagtagaagttccaaggccaaagaaatcaagaagcaagaaggagaaagaggacgaggaagaggtgttactgataaaaggaatagagctagatagagagaatttcgtgaagtttgatgtgtacatcaacgacgaagattattcagtgagtaggcctaagaatagtgagtttgcaggaagctttgtgaacgtaccacacaagcatatgaaagaaatgaagacgaagaccaatctgaggttggcgataaatgagctgttagaggacttgggagccgaagatgatgagagtgtgatcgtgactatagtccctcgtgctgggggcgatgatgtcaccattggtggaattgagatcgagtttgtttccgattgatcccacctttcaatgattatccattatatgtatgtatcaggtaagtcacatctttatgtgactaatggaaaatgtgagacttctctgtactctcccgtcaagtcttttattaatttagagctgtttggtttaaatttaa

SEQ ID NO: 11 - DNA, 1692 bp, Vitis vinifera
  source 1..1692: mol_type = unassigned DNA
  misc_feature 538..642: function = Tyrosinase Domain
atggcttctttgccttggtcgctcacaacctccaccgccatcgccaacaccaccaacatttcagccttgccaccttctcccttgtttcacagggtttctcatgtccccatagccagaaaccgaagccgcagatttgctcctagtaaggtgtcgtgcaatgctgcgaatggtgatccaaactcggattctacctccgacattcgagaaacttccccagggaagttagataggaggaatgtgcttcttggcataggagggctgtatggtgctgctgccggtctcggcgccactaagccattagcctttggggctcccatccaggcaccggatatacgcaagtgtggtaccgccaccgtgcctaatggtgtaattcccacaaattgttgcccgccagtcaccacaaagcctatacctttccagctaccttcctcagattcccccatgcgtaccaggccagctgctcacttggtcagcaaagagtacttagccaagtataaaaaggccattgagctgcagaaagctcttcctgatgatgatccgcgtagtttcaagcaacaggccaatgtccattgcgcctattgccaaggggcttatgatcaggttgggtataccgacctagaactccaggttcatgcttcatgggtttctcatgtccccatagccagaaaccgaagccgcagatttgctcctagtaaggtgtcgtgcaatgctgcgaatggtgatccaaactcggattctacctccgacattcgagaaacttccccagggaagttagataggaggaatgtgcttcttggcataggagggctgtatggtgctgctgccggtctcggcgccactaagccattagcctttggggctcccatccaggcaccggatatatccaagtgtggtaccgccaccgtgcctgatggtgtaacgcccacaattgttgcccgccagccagctgctcacttggtcagcaaagagtacttagccaagtataaaaaggccattgagctgcagaaagctcttcctgatgatgatccgcgtagtttcaagcaacaggctaatgtccattgcacctattgccaaggggcttatgatcaggttgggtataccgacctagaactccagaaagtcaaggtctcggactgtgtcgacacttccaagctgagataccaatatcaggatattncaatcccatggctaccaaaaaatacgaaggccaaagcgaagacgaccaccaaaagttccaagtcgggagtagcgaaagcggccgaactcccaaagacgacgatcagcagcatcggagacttcccaaaagctcttaactcagtgataagagtagaagttccaaggccaaagaaatcaagaagcaagaaggagaaagaggacgaggaagaggtgttactgataaaaggaatagagctagatagagagaatttcgtgaagtttgatgtgtacatcaacgacgaagattattcagtgagtaggcctaagaatagtgagtttgcaggaagctttgtgaacgtaccacacaagcatatgaaagaaatgaagacgaagaccaatctgaggttggcgataaatgagctgttagaggacttgggagccgaagatgatgagagtgtgatcgtgactatagtccctcgtgctgggggcgatgatgtcaccattggtggaattgagatcgagtttgtttccgattga

SEQ ID NO: 12 - protein, 562 aa, Vitis vinifera
  REGION 180..214: note = Tyrosinase Domain
MASLPWSLTTSTAIANTTNISALPPSPLFHRVSHVPIARNRSRRFAPSKVSCNAANGDPNSDSTSDIRETSPGKLDRRNVLLGIGGLYGAAAGLGATKPLAFGAPIQAPDIRKCGTATVPNGVIPTNCCPPVTTKPIPFQLPSSDSPMRTRPAAHLVSKEYLAKYKKAIELQKALPDDDPRSFKQQANVHCAYCQGAYDQVGYTDLELQVHASWVSHVPIARNRSRRFAPSKVSCNAANGDPNSDSTSDIRETSPGKLDRRNVLLGIGGLYGAAAGLGATKPLAFGAPIQAPDISKCGTATVPDGVTPTIVARQPAAHLVSKEYLAKYKKAIELQKALPDDDPRSFKQQANVHCTYCQGAYDQVGYTDLELQKVKVSDCVDTSKLRYQYQDIIPWLPKNTKAKAKTTTKSSKSGVAKAAELPKTTISSIGDFPKALNSVIRVEVPRPKKSRSKKEKEDEEEVLLIKGIELDRENFVKFDVYINDEDYSVSRPKNSEFAGSFVNVPHKHMKEMKTKTNLRLAINELLEDLGAEDDESVIVTIVPRAGGDDVTIGGIEIEFVSD

SEQ ID NO: 13 - DNA, 2195 bp, Vitis vinifera
  source 1..2195: mol_type = unassigned DNA
atggataatgggagatttccatggggggcctatacagtgcagaaaatcagttttccttgtccttttcagctttttatttatttacttttctacggcaagtgttttctcgacatccctaaggtaagaatacgtactacctaccccagttcattctgtacagagactgaggccatagagcggtctcacttatcacccgtaactcctctaaagctatggcttctttgccttggcagctcacaacctccaccgccaccgccaccgccaccgtcaccacctccaacatttcggccttgtcaccttctcccttgtttcacagggtttctcgggggagtcagaaatcgaaaccacagatctgctcctagtaaagtgtcgtgcaattctacaaatggtgatccaaactcggattctacctccgacattcgagaaacttccccagggaagttagataggaggaatgtgcttcttggcataggagggctgtatggtgccgctggcggcctcggcgccactgagccattagcctttggggctcccatccaggcaccggatgtaaacaagtgtggtgccgccaccgtgcctgagggtgtaacgcccacaaattgttgcccgccagccaccacaaagattatagatttccagctaccttccccagattcccccatgcgcattaggccagctgctcacttggtcagcaaagagtacatagccaagtataaaaaggccattgagctaatgaaagctcttcctgatgatgatccacggagcttcatgcagcaggccaatgtccattgcgcctattgcgatggggcttatgatcaggttgggtttccggacctagagctccaggttcatttttcatggcttttcttccctttccaccgtttctattgctactttcatgagagaattcttgcaaaactgattaacgatcccaccttcgctttgccctattggagctgggattcccctcctcctggaatgcaaatgccgggcatctatgctgattccacatcaacactctacgacgcgaagcgcaacgccaaccacctgcctccgactgtaatcgatctcgactacgatttcaccgaacccacaatcccagtttcggaattagttgccaacaacctgtcaatcatgtaccggcaagttgtgtcgggtgccacgactgctaagctattctttggtgccccataccgtgctggcgatcagcctgaccccggaccgggtaccattgagaacgtcccacatggtccagtccacatatggactggactttttgacgagcctcatgaggacatgggaaacttctatactgccggcagagaccccatatttttcggtcaccactccaacgtcgaccgtatgtggacgctatggaaagctataggaggtaaaaatagaacggaattcacggacccagattggctaaacgccgggttcgtcttctacgacgagaacgcccaacttgttcgcgtcaaggtccgagactgcctcgaccatctcaagctgagatacaaatatcaggatattcctattccatggctaaaaaaaagtacgcagcccaaagcgaagacgaccaccaaaagttccaagtcgggagtagcgaaagcggcggaactcccaaagacgacgatcagcagcagtggaggcttcccaaaacctcttaactcagtaataagagtagaagttccgaggccaaagaaatcaagaagcaagaaggagaaagaggatgaagaagaggtgttgctgataaaaggaatagagctagatagagatgatttcgtgaagtttgatgtgtacatcaacgacgaagattattcagtgagtaagcctagtaatagtgagttcgcaggaagcttggtgaacgtaccacacaagcatacgcagagaatgaagacaaagaccagcctgaggttagcgataaatgaattactagataacttgggggctgaagatgatgagagtgtgatcgtgactatagtccctcgtgctgggaccgatggtgtcaccattggtggaattgagatcgagtttgtttccgattgatcccacctttcgattcttatccattatatgtatccggtcacgtgtgtttcgaaatcaatgaaggtcatttctacgcattatgtatcatgtaaatcacatcagtatgtgactaatggaaaatgtgaaatttctttctactttccccttcc

SEQ ID NO: 14 - DNA, 2046 bp, Vitis vinifera
  source 1..2046: mol_type = unassigned DNA
  misc_feature 757..1377: function = Tyrosinase Domain
atggataatgggagatttccatggggggcctatacagtgcagaaaatcagttttccttgtccttttcagctttttatttatttacttttctacggcaagtgttttctcgacatccctaaggtaagaatacgtactacctaccccagttcattctgtacagagactgaggccatagagcggtctcacttatcacccgtaactcctctaaagctatggcttctttgccttggcagctcacaacctccaccgccaccgccaccgccaccgtcaccacctccaacatttcggccttgtcaccttctcccttgtttcacagggtttctcgggggagtcagaaatcgaaaccacagatctgctcctagtaaagtgtcgtgcaattctacaaatggtgatccaaactcggattctacctccgacattcgagaaacttccccagggaagttagataggaggaatgtgcttcttggcataggagggctgtatggtgccgctggcggcctcggcgccactgagccattagcctttggggctcccatccaggcaccggatgtaaacaagtgtggtgccgccaccgtgcctgagggtgtaacgcccacaaattgttgcccgccagccaccacaaagattatagatttccagctaccttccccagattcccccatgcgcattaggccagctgctcacttggtcagcaaagagtacatagccaagtataaaaaggccattgagctaatgaaagctcttcctgatgatgatccacggagcttcatgcagcaggccaatgtccattgcgcctattgcgatggggcttatgatcaggttgggtttccggacctagagctccaggttcatttttcatggcttttcttccctttccaccgtttctattgctactttcatgagagaattcttgcaaaactgattaacgatcccaccttcgctttgccctattggagctgggattcccctcctcctggaatgcaaatgccgggcatctatgctgattccacatcaacactctacgacgcgaagcgcaacgccaaccacctgcctccgactgtaatcgatctcgactacgatttcaccgaacccacaatcccagtttcggaattagttgccaacaacctgtcaatcatgtaccggcaagttgtgtcgggtgccacgactgctaagctattctttggtgccccataccgtgctggcgatcagcctgaccccggaccgggtaccattgagaacgtcccacatggtccagtccacatatggactggactttttgacgagcctcatgaggacatgggaaacttctatactgccggcagagaccccatatttttcggtcaccactccaacgtcgaccgtatgtggacgctatggaaagctataggaggtaaaaatagaacggaattcacggacccagattggctaaacgccgggttcgtcttctacgacgagaacgcccaacttgttcgcgtcaaggtccgagactgcctcgaccatctcaagctgagatacaaatatcaggatattcctattccatggctaaaaaaaagtacgcagcccaaagcgaagacgaccaccaaaagttccaagtcgggagtagcgaaagcggcggaactcccaaagacgacgatcagcagcagtggaggcttcccaaaacctcttaactcagtaataagagtagaagttccgaggccaaagaaatcaagaagcaagaaggagaaagaggatgaagaagaggtgttgctgataaaaggaatagagctagatagagatgatttcgtgaagtttgatgtgtacatcaacgacgaagattattcagtgagtaagcctagtaatagtgagttcgcaggaagcttggtgaacgtaccacacaagcatacgcagagaatgaagacaaagaccagcctgaggttagcgataaatgaattactagataacttgggggctgaagatgatgagagtgtgatcgtgactatagtccctcgtgctgggaccgatggtgtcaccattggtggaattgagatcgagtttgtttccgattga

SEQ ID NO: 15 - protein, 681 aa, Vitis vinifera
  REGION 253..459: note = Tyrosinase Domain
MDNGRFPWGAYTVQKISFPCPFQLFIYLLFYGKCFLDIPKVRIRTTYPSSFCTETEAIERSHLSPVTPLKLWLLCLGSSQPPPPPPPPPSPPPTFRPCHLLPCFTGFLGGVRNRNHRSAPSKVSCNSTNGDPNSDSTSDIRETSPGKLDRRNVLLGIGGLYGAAGGLGATEPLAFGAPIQAPDVNKCGAATVPEGVTPTNCCPPATTKIIDFQLPSPDSPMRIRPAAHLVSKEYIAKYKKAIELMKALPDDDPRSFMQQANVHCAYCDGAYDQVGFPDLELQVHFSWLFFPFHRFYCYFHERILAKLINDPTFALPYWSWDSPPPGMQMPGIYADSTSTLYDAKRNANHLPPTVIDLDYDFTEPTIPVSELVANNLSIMYRQVVSGATTAKLFFGAPYRAGDQPDPGPGTIENVPHGPVHIWTGLFDEPHEDMGNFYTAGRDPIFFGHHSNVDRMWTLWKAIGGKNRTEFTDPDWLNAGFVFYDENAQLVRVKVRDCLDHLKLRYKYQDIPIPWLKKSTQPKAKTTTKSSKSGVAKAAELPKTTISSSGGFPKPLNSVIRVEVPRPKKSRSKKEKEDEEEVLLIKGIELDRDDFVKFDVYINDEDYSVSKPSNSEFAGSLVNVPHKHTQRMKTKTSLRLAINELLDNLGAEDDESVIVTIVPRAGTDGVTIGGIEIEFVSD

SEQ ID NO: 16 - DNA, 5504 bp, Vitis vinifera
  source 1..5504: mol_type = unassigned DNA
atgggaaaatcatctctatttcctttgaatttttttttaaaaaaattagtttggaatttcaattttttgtaatattaattttttcttttaatttgggattttttaaaaaaaaaattattttttataatattaattttaaaaaaacttttgaatttttttttaaatgggaaaatagtctcttgaagagaaaatttcccttttttaaattagtttccatttttatgtaatattaagggattttttttaatttttttttaaattattttttataatattaattttttttaaaaaaaattcaaagggaaatcgtctcttgaagagatgatttccctttaatattaattttggattttgttttttttttttttaaatttaattattttttataatattaattttaaattttttttttaaattagtttggaaaatatatattttttttttaaatggtaaattaaatttttttttttaagggaaattgtctcttcaagaggaaatttatttattttttaaaatggtaaattaaattttttttaaggcaagagacgatttcccttttggaaattttttttttcttttttttaaattagtttgaaaaatatatatattttttaaaatggtaaattaaaatttttttaaagggaaatcatctcttgaagagacagatttcccttttggattttttttttttttaaattagtttggaaaattttttttttaaatggtaaatttaaaaaaaatttaaggaaatttttttttttttttaaatggtaaatttaaaattttttttaagggaaatcgtcatttcaagagatgatttcccttttggaattttttttttttaaattagtttggaaaatatatatattttttaaaatggtaaattaaattttttttttttttaagggaaatcggctcttgaagagacgatttcccttttggaatttttttttaaaaattagtttggaaaatatatatatttttttaaatggtaaattaaaatatttttaaaggaaaatcgtctcttcaagagacacttttggaatttttttttgttttttaaattagtttggaattttttttaaatggtaaattaaaatttttttaagggaattttggatttttttttttttaaatgaagagaagatttccctttgaatttctttttatttccatttatgattttaaaaccgtcttttttaaagacgattttggttttaaaaaaaaaaaattttagcgtgcctctctctcctacctacaaaatgttcttggaataactttttctactaagacaatttaagaataattttttttaattaccgttatcttgtgaaaagtccccgccaccaccaccaacatttcggccttgtcaccttcccccttgtttctcagggtttctcatgtccccatcgtcagaaatcgaaaccacagatctgctccaagtaaaatgacttgcaattctgcaaatggtgatccaaactcggattccacctccgacattcgaggaactatgccaagaagcacattcctcctatctaacttcccttccctagggaagttagattggaggaatgtgcttcttggcataggagggctgtatggtgccgctggcagcctcgcgccactgagccattagccttcggagctcccatccaggcaccggatgtaactaagtgtggtgtcgccaccgtgcctgcgggtgtaacggccacaaattgttgcccgccagccaccacaaagattatagatttccagctgccttccccagattccctcctgcgcattaggccagctgctcacttggtcatcaaagagtacagagccaagtataaaaaggccattcagctaatgaaagctcttcctgatgatgatccatggagcttcaggcaacaggccagtgtccgttgcgcctattgcaatggggctgatgatcaggttgggtttccggatctagaaatccaggttcatgcttcatggcttttcttccctttccaccgtttatatgcctgcttccatgagagaattcttgcagaactgattaacgatcccacctccgctttgccctattggagctgggattcccctcctggtatgcaaatgccggccatctatgctgcttccacatcaacacgctacgacgcgaagcgcaacgccaaacacctgcctccgactgtaatcaatcttgactacgatttcaccgaatccacatccccgatttggaaatagttgccaacaatctgtcaatcgtataccggcaagttgtgtcgggtgccacgactgctaagcttttctttggtagcccataccgtgccggagatcagcctgaccctggagagggtaccattgatagcgtcccggccacatggtccagtccacatatggactggtctttttgacaagcctcatgaggacatgggaaacttctatactgctggcaaagaccccatattcttcggtcaccacgccaacgtcgaccgtatgtggatgctatggaaagctataggaggtaaaaatagaactgaattcacggacccagattcgctaaacgccgggttcgtcttctacgatgagaacgcccaacttgttcgagtcaaggtccgagactgcctggaccatctcaaactgagatacaaatatcaggatattcctattccatagctaaaaaaaagtacgcagcccaaagcgaagacgaccaccaaaagttccaagtcgggagtagcgaaagcggcggaactcccaaagacgacgatcagcagcagtggaggcttcccaaaacctcttaactcagtaataagagtagaagttccatggccaaagaaatcaagaagcaagaagaagaaagaggatgaagaagaggtgttgctgaagactattcagtaagcccagcaatagtgagttaacaggaagctttgttaacgtaccacaagcataagcagagaatgaagacgaagaccagcctgctcagttagcgataaagaggactggggctgaagacgatgacagttgtccctcatgctcggactgatgatgccaccattggtggaattgagatcgagtttatttctgattgattccacctttcaattatcatccataatatatatccgatcaaggaaaagtccatgcggtactgcaccaatgcagtactacacccaatgcagtactacaccaaatgcagtactgtagcctactccgaattcagtactacaccaaatgcagtagtaccaaaacttttttcatctgttgacttggccatccattttaattttttttttttaaaaaagatatgaccggtaccaaagattgagggtatggtaccaaaacatgtggcctattgttattggtggaagattcagggtattgggtacccaagcatgacctgatcatgtgtttttcaaaatcaatgaaggtcatttccgcggatgatgcatcatgtatgtgaaattgctttctaccttcccctccaagtcttttgctaatctagagctgttttctttatattaagggatttttaattagggatagtttcttgtgccttccttctcctttacatcgaaattgaagttgaagccgaagtcaattaaattcataatttttataagacattattgatattataaaaattttattaaatataaaataaatttttatataagttcaaatataatttttataatacaaaatttattttacttttgtgttagttattttttttttttttaagttataaggtctttaaataaaattaatcaaatagatatgtacacattcaaattctaattatagggcttagaaaatagtttttggtttcttaaaaagtcgatccaagtagatcctacatatatcacgggatattcaagatcatatgagtcgaaatttcaaattaggtccggaactagaggcttatttgggcaatgtctttcttgtctctagttttcaagtatggttcctaaatagacctttaaagttcttgagatagaaaattagatatagtagaaaattatagtttataattttttttttaatataaaaaagaaaataagaggaacaaaatcgaaaaagcatcttaaacaaaatttatggagctgaaccaaccttgtgtgcttgtgtttcttctcccttattttcccttttatcaaacttttcaaaattcaaaagtggctttgaaaaccttgttttgcatagagagctagctgattttaatcagagccacaagcaaaaatatcaaatccattgcctaattaatcatttgacttactatccgatttgggttttggatttaatgtgaacaattttcaaactctaactacttgaaacccaattctagattgggcctggcccaagttgaagcggcctttttaacccatgctgaaaggttgctgcttcaatttgaatacctcgcgagggtattgtctttgaaagaacaactttactttagatttatttatatacatagagagagatatgaaaatagatttaaagttaataaattatttttacatgttttttgaaatttgttatacatatttttttatcttccatataaaaattaaataattttaaaatgtataaatgtctaacaaatttgaattatatcttctttttttaatttgaattatatttaattttttatatatttcttattgtaaatggaacttaagaaaacattttacttatcatttctgttctttttattttccttcaaataaaacaaatttaccatttttttacttagtattttccttggaatcaaacatagttcaactctgtcttagaaagaaagaaaaataaatgaaatactagatatgatgatcttcaaagactcataaatcgaatcttaagaaataagtaaaatatttgaggataaatatcaaatttcaatttaaaataatttatcatagttatgagttaaacttgatcattatccttgaaattatttaaggggatatggttgtttcatataggatatcatgatgtccttattgagagtttccatgttgtagcatgttgaataaaataattgtaggaaactttggattacttcattctccgaccttagttcattaaggtgcatgcaatcattaaggtgaagtagtgggatttcgggtttttgagaatatggggaagaagaggagaggttagggattcatggaatttcatgatttttggagaagtgaaagaagttaaggtttcaagaaattttgagaatgaggaagaaaaggaaaggttagatgattcatgggatttttagagaaaaaacgagattagggcttctagagatcttgagaaaagaggtagaattggttaagactttgttggaattttgggtttgtgtctttctcttctttgatttttttttttttcacatgtgggagacttagctggcaatgtggatggcattttttaaattgaattacacacaagctaaaagtatatgaggaaggattttagatcgaatcataaaagttctgttgttcgtgtagatatcttcgaagtctgcttga

SEQ ID NO: 17 - RNA, 20 nt, synthetic construct
  source 1..20: mol_type = other RNA
gcctgttgcatgaagctccg

SEQ ID NO: 18 - RNA, 20 nt, synthetic construct
  source 1..20: mol_type = other RNA
tcattgcgcctactgcaatg

SEQ ID NO: 19 - RNA, 20 nt, synthetic construct
  source 1..20: mol_type = other RNA
gatcattaatcaactttccc

SEQ ID NO: 20 - RNA, 20 nt, synthetic construct
  source 1..20: mol_type = other RNA
ggcaaaggcgaatggatcag

SEQ ID NO: 21 - RNA, 20 nt, synthetic construct
  source 1..20: mol_type = other RNA
atgaacctggagttctaggt

SEQ ID NO: 22 - RNA, 20 nt, synthetic construct
  source 1..20: mol_type = other RNA
tgaagtagagatagtaacgg

SEQ ID NO: 23 - RNA, 20 nt, synthetic construct
  source 1..20: mol_type = other RNA
ggggaactagcatagatggt

SEQ ID NO: 24 - RNA, 20 nt, synthetic construct
  source 1..20: mol_type = other RNA
gtgatcgatctcgactacga

SEQ ID NO: 25 - DNA, 20 bp, synthetic construct
  source 1..20: mol_type = other DNA
gatgatgacccgcgtagttt

SEQ ID NO: 26 - DNA, 24 bp, synthetic construct
  source 1..24: mol_type = other DNA
cccttggcaataggtgcaatggac

SEQ ID NO: 27 - DNA, 22 bp, synthetic construct
  source 1..22: mol_type = other DNA
gaagcatgaacctggagttcta

SEQ ID NO: 28 - DNA, 19 bp, synthetic construct
  source 1..19: mol_type = other DNA
caaacatcagccgccaaag

SEQ ID NO: 29 - DNA, 24 bp, synthetic construct
  source 1..24: mol_type = other DNA
cgactacacctcaccgacagcaat

SEQ ID NO: 30 - DNA, 22 bp, synthetic construct
  source 1..22: mol_type = other DNA
gccggtacataatggagagatt

SEQ ID NO: 31 - DNA, 22 bp, synthetic construct
  source 1..22: mol_type = other DNA
cacaatccctgatgacgaacta

SEQ ID NO: 32 - DNA, 24 bp, synthetic construct
  source 1..24: mol_type = other DNA
aaacaaattgtgtcgggtgccacg

SEQ ID NO: 33 - DNA, 20 bp, synthetic construct
  source 1..20: mol_type = other DNA
gcggtatgggtaaccaaaga

SEQ ID NO: 34 - DNA, 22 bp, synthetic construct
  source 1..22: mol_type = other DNA
cctagtaaggtgtcgtgcaatg

SEQ ID NO: 35 - DNA, 24 bp, synthetic construct
  source 1..24: mol_type = other DNA
tgcgaatggtgatccaaactcgga

SEQ ID NO: 36 - DNA, 23 bp, synthetic construct
  source 1..23: mol_type = other DNA
ccaagaagcacattcctcctatc

SEQ ID NO: 37 - DNA, 20 bp, synthetic construct
  source 1..20: mol_type = other DNA
tcttccctttccaccgtttc

SEQ ID NO: 38 - DNA, 24 bp, synthetic construct
  source 1..24: mol_type = other DNA
taacgatcccaccttcgctttgcc

SEQ ID NO: 39 - DNA, 20 bp, synthetic construct
  source 1..20: mol_type = other DNA
gggaatcccagctccaatag

SEQ ID NO: 40 - DNA, 21 bp, synthetic construct
  source 1..21: mol_type = other DNA
ggcgttctcatcgtagaagac

SEQ ID NO: 41 - DNA, 24 bp, synthetic construct
  source 1..24: mol_type = other DNA
cgtttagcgaatctgggtccgtga

SEQ ID NO: 42 - DNA, 21 bp, synthetic construct
  source 1..21: mol_type = other DNA
cgaccgtatgtggatgctatg

SEQ ID NO: 43 - DNA, 21 bp, synthetic construct
  source 1..21: mol_type = other DNA
ggacccatcagaaatcccttg

SEQ ID NO: 44 - DNA, 24 bp, synthetic construct
  source 1..24: mol_type = other DNA
aacaaccgagaaggcttctgcaca

SEQ ID NO: 45 - DNA, 20 bp, synthetic construct
  source 1..20: mol_type = other DNA
tcttggcaccaccctttatg

SEQ ID NO: 46 - DNA, 1290 bp, Vitis vinifera
  source 1..1290: mol_type = unassigned DNA
atggcgctctattctctcttcagatccgcgccttccgccgccgccgccgccgccgctactgtggtggattcttctcgtccttctgatcgatgcaaggtgtcttgcattggtttcaatagcaatgcggactcactgaaggttcagaccaccatatttggtactccttgtattggaaattcatcttccttacagaaatgcagtgccaggagtctccagcccatcaaagccacagccacagaattgcctcctacagttcaaaagtcatggagcagtgggcagacaaagattggaatcaatggttttggtcgaattggaaggttggttttacgagtagcaacctctagggatgatattgatgtggtagcagtcaatgatccatttgttgatgccaaatacatggcttacatgttcaagtatgattctactcatggagttttcaagggaaccatcaaggtattggatgagtccactttggaaatcaatggcaagcagataaaagttacaagcaaaagggacccatcagaaatcccttggggtgattatgggattgattatgttgttgaatcttctggagttttcacaacaaccgagaaggcttctgcacacataaagggtggtgccaagaaagtggtaatttcagctccatcagctgatgcaccgatgtttgtggtaggagtaaatgagacttcatacaagccaagcatgaatattgtttctaatgcaagctgtaccaccaattgccttgctcctcttgccaaggttgttcatgaggaatttggtatcgttgagggtttaatgacaactgtgcatgccactacagccacacagaagaccgtggatgggccttcaatgaaggattggcgagggggccgaggtgcttcacaaaatatcatcccaagctccactggtgctgcaaaggctgttggaaaagtccttccagaactcaacggaaagctcactggaatggccttccgcgtcccaacacctaatgtctctgttgtggacttaacttgtcgacttaagaagagtgctacatatgaagatgtcaaagcagccgtaaagtatgcctcagaggggccattgaagggtattcttggatacacggatgaggatgttgtctccaatgattttgttggtgactcaaggtcaagtatatttgatgcaaaggccgggatagggctgagtgcttccttcatgaagctcgttgcatggtatgacaacgaatggggatacagcaaccgagtgctggacctcatagagcacatggcattggtggcggccagcaaatga

SEQ ID NO: 47 - protein, 429 aa, Vitis vinifera
MALYSLFRSAPSAAAAAAATVVDSSRPSDRCKVSCIGFNSNADSLKVQTTIFGTPCIGNSSSLQKCSARSLQPIKATATELPPTVQKSWSSGQTKIGINGFGRIGRLVLRVATSRDDIDVVAVNDPFVDAKYMAYMFKYDSTHGVFKGTIKVLDESTLEINGKQIKVTSKRDPSEIPWGDYGIDYVVESSGVFTTTEKASAHIKGGAKKVVISAPSADAPMFVVGVNETSYKPSMNIVSNASCTTNCLAPLAKVVHEEFGIVEGLMTTVHATTATQKTVDGPSMKDWRGGRGASQNIIPSSTGAAKAVGKVLPELNGKLTGMAFRVPTPNVSVVDLTCRLKKSATYEDVKAAVKYASEGPLKGILGYTDEDVVSNDFVGDSRSSIFDAKAGIGLSASFMKLVAWYDNEWGYSNRVLDLIEHMALVAASK

SEQ ID NO: 48 - DNA, 861 bp, Vitis vinifera
  source 1..861: mol_type = unassigned DNA
atgggtggaaactatggcaaccgagagatcagagtgccttttgcacattttcttatgggaaggtctggtagatttaacttagatgtcccatattcatctcgtcttcaacagcaacaacaacatgcttcatcaattggaagcggtggaccaccttccataggtccattactgctgccaccgcaactgcctcaatctctttttcctcttcacatgggttcagccgctcctcgtcccgccgtgatcattaactacggcaccaggtatattaaaatgggtttttccggtaacgtggagccgtgcttcattgccccgacagttgttgctgtcaacgagtcgtttttgaatcaaaatcggagcaccaccaacccaagctatccgattgttcacggccaggtttataattgggatgtcatggagagattttggcagcagggtatatttaattatttgagatgtgatcaggaggatcactactttcttttggcgggaagtccactcactgccccggagagccgtgaatacactggtgaaattatgtttgaaaccttcaaatgttcctggtctttatattgcagtgcagccggtactcgcccatgtgacaggatacactacatccaagtggcagtagatgacaaggatggtgcaatttctggggagataagaagcaatggccttaaagcctacgttcatctatatcttgctgcactcaatggtgattggaaaagtgcaaaagcattcctggagtcaaatccacaaacagtgagaacgaggatcacaaggctcttagaaactgctcttcatattgctgctatggcaggatttgcatctgcaagtgatagaggtatcaggagaagcagataa

SEQ ID NO: 49 - protein, 286 aa, Vitis vinifera
MGGNYGNREIRVPFAHFLMGRSGRFNLDVPYSSRLQQQQQHASSIGSGGPPSIGPLLLPPQLPQSLFPLHMGSAAPRPAVIINYGTRYIKMGFSGNVEPCFIAPTVVAVNESFLNQNRSTTNPSYPIVHGQVYNWDVMERFWQQGIFNYLRCDQEDHYFLLAGSPLTAPESREYTGEIMFETFKCSWSLYCSAAGTRPCDRIHYIQVAVDDKDGAISGEIRSNGLKAYVHLYLAALNGDWKSAKAFLESNPQTVRTRITRLLETALHIAAMAGFASASDRGIRRSR

SEQ ID NO: 50 - DNA, 20 bp, synthetic construct
  source 1..20: mol_type = other DNA
gcagaaagctcttcctgatg

SEQ ID NO: 51 - DNA, 20 bp, synthetic construct
  source 1..20: mol_type = other DNA
gcatatacatgccatcaggg

SEQ ID NO: 52 - DNA, 24 bp, synthetic construct
  source 1..24: mol_type = other DNA
tccaccgttactatctctacttca

SEQ ID NO: 53 - DNA, 19 bp, synthetic construct
  source 1..19: mol_type = other DNA
catgattgccagattgtcg

SEQ ID NO: 54 - DNA, 21 bp, synthetic construct
  source 1..21: mol_type = other DNA
aacctctagctccaaaagtgg

SEQ ID NO: 55 - DNA, 23 bp, synthetic construct
  source 1..23: mol_type = other DNA
aatctatgatcttgctgctcact

SEQ ID NO: 56 - DNA, 23 bp, synthetic construct
  source 1..23: mol_type = other DNA
agtgagcagcaagatcatagatt

SEQ ID NO: 57 - DNA, 20 bp, synthetic construct
  source 1..20: mol_type = other DNA
catgaattgtggacttggag

SEQ ID NO: 58 - DNA, 20 bp, synthetic construct
  source 1..20: mol_type = other DNA
tccaagtccacaattcatgg

SEQ ID NO: 59 - DNA, 18 bp, synthetic construct
  source 1..18: mol_type = other DNA
tttggcgtcgcgaagctt

SEQ ID NO: 60 - DNA, 20 bp, synthetic construct
  source 1..20: mol_type = other DNA
tccaagtccacaattcatgg

SEQ ID NO: 61 - DNA, 18 bp, synthetic construct
  source 1..18: mol_type = other DNA
tttggcgtcgcgaagctt

SEQ ID NO: 62 - DNA, 20 bp, synthetic construct
  source 1..20: mol_type = other DNA
tccaagtccacaattcatgg

SEQ ID NO: 63 - DNA, 18 bp, synthetic construct
  source 1..18: mol_type = other DNA
tttggcgtcgcgaagctt

SEQ ID NO: 64 - DNA, 19 bp, synthetic construct
  source 1..19: mol_type = other DNA
atggcttctttgccttggt

SEQ ID NO: 65 - DNA, 27 bp, synthetic construct
  source 1..27: mol_type = other DNA
ctatagttttccatatattccacattc

SEQ ID NO: 66 - DNA, 22 bp, synthetic construct
  source 1..22: mol_type = other DNA
caagtgttttctcgacatccct

SEQ ID NO: 67 - DNA, 18 bp, synthetic construct
  source 1..18: mol_type = other DNA
tgtgggttcggtgaaatc

SEQ ID NO: 68 - DNA, 24 bp, Vitis vinifera
  source 1..24: mol_type = unassigned DNA
  misc_feature 1..24: allele = D126-1
tgatgacccgcgtctctacttcaa

SEQ ID NO: 69 - DNA, 25 bp, Vitis vinifera
  source 1..25: mol_type = unassigned DNA
  misc_feature 1..25: allele = D5-1
ccctttccacctatctctacttcaa

SEQ ID NO: 70 - DNA, 25 bp, Vitis vinifera
  source 1..25: mol_type = unassigned DNA
  misc_feature 1..25: allele = D5-2
ccctttccaccgatctctacttcaa

SEQ ID NO: 71 - DNA, 32 bp, Vitis vinifera
  source 1..32: mol_type = unassigned DNA
  misc_feature 1..32: allele = D118
gatgatgatccgcggattactatctttacttc

SEQ ID NO: 72 - DNA, 33 bp, Vitis vinifera
  source 1..33: mol_type = unassigned DNA
  misc_feature 1..33: allele = D117
gatgatgatccgcggagttactatctttacttc

SEQ ID NO: 73 - DNA, 50 bp, Vitis vinifera
  source 1..50: mol_type = unassigned DNA
  misc_feature 1..50: allele = I1-1
agctcttcctgatgatgatccgcggaagcttcatgcaacaggccgatgtt

SEQ ID NO: 74 - DNA, 44 bp, Vitis vinifera
  source 1..44: mol_type = unassigned DNA
  misc_feature 1..44: allele = D126-2
tcttcctgatgatgatccgcgtctctacttcaatgagagaattc

SEQ ID NO: 75 - DNA, 45 bp, Vitis vinifera
  source 1..45: mol_type = unassigned DNA
  misc_feature 1..45: allele = D5-3
gcttttcctccctttccacctatctctacttcaatgagagaattc

SEQ ID NO: 76 - DNA, 45 bp, Vitis vinifera
  source 1..45: mol_type = unassigned DNA
  misc_feature 1..45: allele = D5-4
gcttttcctccctttccaccgatctctacttcaatgagagaattc

SEQ ID NO: 77 - DNA, 60 bp, Vitis vinifera
  source 1..60: mol_type = unassigned DNA
  misc_feature 1..60: allele = I1-2
taatgaaagctcttcctgatgatgatccacggaagcttcatgcagcaggccaatgtccat

SEQ ID NO: 78 - DNA, 48 bp, Vitis vinifera
  source 1..48: mol_type = unassigned DNA
  misc_feature 1..48: allele = D2
ctgatgatgatccacggcttcatgcagcaggccaatgtccattgcgcc

SEQ ID NO: 79 - DNA, 46 bp, Vitis vinifera
  source 1..46: mol_type = unassigned DNA
  misc_feature 1..46: allele = D4
ctgatgatgatccacggtcatgcagcaggccaatgtccattgcgcc

(19) United States
(12) Patent Application Publication
(10) Pub. No.: US 2026/0317776 A1
(43) Pub. Date: Oct. 15, 2026

(54) COMPOSITIONS AND METHODS FOR IMPROVED GRAPES WITH REDUCED POLYPHENOL OXIDASE

(71) Applicant: GREENVENUS LLC, Davis, CA (US)

(72) Inventors: Arianne TREMBLAY, West Sacramento, CA (US); Jeffrey TOUCHMAN, Davis, CA (US); Walter VISS, Waterford, CA (US); Shiv TIWARI, San Jose, CA (US)

(21) Appl. No.: 19/107,500

(22) PCT Filed: Aug. 31, 2023
(86) PCT No.: PCT/US2023/073224

§ 371 Date: Feb. 27, 2025

Related U.S. Application Data
(60) Provisional application No. 63/403,100, filed on Sep. 1, 2022.

(51) Int. Cl. C12N 15/82; A23L 2/02; A23L 19/00; C12N 9/22; C12N 15/11

(52) U.S. Cl. CPC C12N 15/8249; A23L 2/02; A23L 19/00; C12N 9/226; C12N 15/11; C12N 15/8213; C12N 2310/20

(57) ABSTRACT

The present disclosure is directed to grape cells, plants, plant parts, seeds and fruit comprising a mutation in at least one PPO gene, where said mutations reduce the activity of PPO protein compared to a wild-type. The present disclosure is also directed to beverages and food products made from said plants, plant parts, seeds and fruit, and methods for making the same.

This application claims the priority benefit of U.S. Provisional Patent Application Ser. No. 63/403,100, filed Sep. 1, 2022, which is hereby incorporated by reference in its entirety.

### SEQUENCE LISTING

This application contains a computer readable Sequence Listing which has been submitted electronically in XML format and is hereby incorporated by reference in its entirety. The XML file was created on Aug. 31, 2023, is named 257432001151.xml and is 107,922 bytes in size.

### FIELD

The present disclosure is directed to grape plants, cells, and plant parts having one or more polyphenol oxidase (“PPO”) gene mutations. The gene mutations may impart desirable phenotypes, such as reduced post-harvest fruit browning, increased post-harvest fruit shelf life, and increased fruit polyphenolics. Methods of obtaining grape plants, cells, and plant parts having mutated PPO genes are also disclosed.

### BACKGROUND

There is a need in agriculture for technology to rapidly develop unique varieties of non-Genetically Modified Organism (non-GMO) fruits and vegetables with superior consumer appeal and improved profitability to growers. Conventionally, breeding of fruits and vegetables for selected traits is time-consuming, and some fruits and vegetables are difficult to breed and develop or to genetically manipulate without introducing plant pest sequences.

The primary focus for agriculture industries so far have been on agronomic traits such as yield enhancement, pest tolerance, diseases, and herbicide tolerance. The unmet needs of consumers were typically not a top priority, primarily due to the complexity of the problems and uncertainty in market success.

Fruits and vegetables are major sources of nutrients such as vitamins and antioxidants. Traits including visual appeal, aroma, and taste are key factors in consumer likeability.

Polyphenol oxidase (“PPO”), phenylalanine ammonia lyase (“PAL”), and peroxidases are key enzymes in plants that are recently thought to affect multiple consumer traits, such as browning and reducing the levels of polyphenols, which are major antioxidants in fruits and vegetables (Hanuka et al., “The Link between Polyphenol Structure, Antioxidant Capacity and Shelf-Life Stability in the Presence of Fructose and Ascorbic Acid,” Molecules 25(1):225 (2020); Pap et al. “Berry Polyphenols and Human Health: Evidence of Antioxidant, Anti-Inflammatory, Microbiota Modulation, and Cell-Protecting Effects,” Current Opinion in Food Science 42:167-186 (2021)). PPO is among the major contributors in wound-induced browning in most fruits and vegetables and also reduces the nutrient value by degrading polyphenols. Breeding strategies targeting PAL and PPO pathways have been attempted with limited success.

Browning of post-harvest fruit is a major challenge for the wine industry. First, it introduces an undesired color in wine, and second, the byproducts of post-harvest fruit browning produce an undesirable taste, reducing the aroma and antioxidant properties of wine (Harald, “How to Deal with Uninvited Guests in Wine: Copper and Copper-Containing Oxidases,” Fermentation 6(1):38 (2020)).

Post-harvest fruit browning is not just undesirable in the wine industry. The food and beverage industries also face the challenges of undesired color and reduced nutrient value when providing grape fruits and manufacturing grape juices and other grape-related consumer products. There is a need for improved grape varieties and products with reduced browning, increased shelf-life, improved aroma, improved flavor, and increased polyphenolics.

The present disclosure is directed to overcoming these and other deficiencies in the art.

### SUMMARY

One aspect of the present disclosure relates to a grape plant cell comprising one or more loss-of-function mutations of a nucleic acid sequence encoding a polyphenol oxidase (PPO) gene selected from one or more of PPO1, PPO2, PPO3, PPO4, and PPO5, wherein the grape plant cell has reduced polyphenol oxidase gene expression or activity compared to a wild-type grape plant cell.

Another aspect of the present disclosure relates to a grape plant comprising the grape plant cell described herein.

A further aspect of the present disclosure relates to a grape fruit comprising the grape plant cell described herein.

Another aspect of the present disclosure relates to a grape plant, plant part, seed, or fruit propagated from a grape cell, plant, or fruit described herein, wherein the grape plant, plant part, seed, or fruit comprises a loss-of-function mutation in a polyphenol oxidase (PPO) gene.

A further aspect of the present disclosure relates to a beverage made from the grape fruit described herein.

Another aspect of the present disclosure relates to a food product made from the grape fruit described herein.

Yet another aspect of the present disclosure relates to a method of making a grape plant with reduced polyphenol oxidase (PPO) gene expression or activity. This method involves introducing one or more loss-of-function mutations into a nucleic acid sequence encoding a polyphenol oxidase (PPO) gene selected from one or more of PPO1, PPO2, PPO3, PPO4, and PPO5 of a grape plant cell, wherein the grape plant cell has reduced polyphenol oxidase gene expression or activity compared to a wild-type grape plant cell. The method further involves regenerating a plant from the grape plant cell.

The present disclosure relates to the development of grape cells, grape plants, grape plant parts, grape seeds, and grape fruits, as well as food and beverages made therefrom, having improved traits, including reduced browning, increased shelf life, and increased nutritional value (e.g., increased levels of polyphenolics). Described herein is the identification of six polyphenol oxidase (PPO) genes in grape. The various PPO genes were found to be expressed differently and respond differently to crushing damage. PPO2 was found to the be the most highly expressed gene. Using the genetic approaches described in the present disclosure, grape plant cells and grape plants were produced with loss-of-function mutations in various combinations of specific PPO genes resulting in significantly reduced PPO enzymatic activity.

### BRIEF DESCRIPTION OF THE DRAWINGS

FIG. 1 is a graph of PPO1, PPO2, PPO3, PPO4, PPO5, and PPO6 gene expression (“GE”) relative to expression of GAPDH in multiple varieties of control (non-crushed) grapes including a red seeded variety, a black seedless variety, a green seedless variety, and a green Cotton Candy variety. An asterisk (*) indicates only one biological replicate was used. Three biological replicates were measured for all other samples.

FIG. 2 is a graph of PPO1-PPO6 gene expression relative to expression of GAPDH in multiple varieties of grapes wounded by crushing.

FIG. 3 is a graph of PPO1 gene expression in control versus crushed grapes relative to expression of GAPDH in multiple varieties of grapes.

FIG. 4 is a graph of PPO2 gene expression in control versus crushed grapes relative to expression of GAPDH in multiple varieties of grapes.

FIG. 5 is a graph of PPO3 gene expression in control versus crushed grapes relative to expression of GAPDH in multiple varieties of grapes.

FIG. 6 is a graph of PPO4 gene expression in control versus crushed grapes relative to expression of GAPDH in multiple varieties of grapes.

FIG. 7 is a graph of PPO5 gene expression in control versus crushed grapes relative to expression of GAPDH in multiple varieties of grapes.

FIG. 8 is a graph of PPO6 gene expression in control versus crushed grapes relative to expression of GAPDH in multiple varieties of grapes.

FIGS. 9A-B are alignments of the PPO1 coding sequence with the genome edited alleles of SEQ ID NOs:68-70. FIG. 9A is an alignment of nucleotides 531-680 of the PPO1 coding sequence (SEQ ID NO:2) with the 126-nucleotide deletion allele 1 of PPO1 (D126-1, SEQ ID NO:68). FIG. 9B is an alignment of nucleotides 651-680 of the PPO1 coding sequence (SEQ ID NO:2) with the 5-nucleotide deletion alleles 1 and 2 of PPO1 (D5-1, SEQ ID NO:69; and D5-2, SEQ ID NO:70).

FIGS. 10A-B are alignments of the PPO2 coding sequence with the genome edited alleles of SEQ ID NOs:71-73. FIG. 10A is an alignment of nucleotides 511-660 of the PPO2 coding sequence (SEQ ID NO:5) with the 118-nucleotide deletion allele of PPO2 (D118, SEQ ID NO:71), and the 117-nucleotide deletion allele of PPO2 (D117, SEQ ID NO:72). FIG. 10B is an alignment of nucleotides 501-550 of the PPO2 coding sequence (SEQ ID NO:2) with the 1-nucleotide insertion allele of PPO2 (I1-1, SEQ ID NO:73).

FIGS. 11A-B are alignments of the PPO3 coding sequence with the genome edited alleles of SEQ ID NOs:74-76. FIG. 11A is an alignment of nucleotides 81-250 of the PPO3 coding sequence (SEQ ID NO:8) with the 126-nucleotide deletion allele 2 of PPO3 (D126-2, SEQ ID NO:74). FIG. 11B is an alignment of nucleotides 201-250 of the PPO3 coding sequence (SEQ ID NO:8) with the 5-nucleotide deletion alleles 3 and 4 of PPO3 (D5-3, SEQ ID NO:75; and D5-4, SEQ ID NO:76).

FIGS. 12A-B are alignments of the PPO5 coding sequence with the genome edited alleles of SEQ ID NOs:77-79. FIG. 12A is an alignment of nucleotides 731-790 of the PPO5 coding sequence (SEQ ID NO:14) with the 1-nucleotide insertion allele (I1-2) of PPO5 (I1-2, SEQ ID NO:77). FIG. 12B is an alignment of nucleotides 651-700 of the PPO5 coding sequence (SEQ ID NO: 14) with the 2-nucleotide deletion allele and 4-nucleotide deletion allele of PPO5 (D2, SEQ ID NO:78; and D4, SEQ ID NO:79).

FIG. 13 is a graph showing PPO enzymatic activity over time in wild-type Colombard grape variety leaves (22300) versus grape genome edited lines 22300-1, -8, -11, -13, -17, and -35.

FIG. 14 is a photograph of the PPO enzymatic activity assay taken 18 hours after the start of the assay.

FIG. 15 is a graph showing PPO specific activity in wild-type Colombard grape variety leaves (22300) versus grape genome edited lines 22300-1, -8, -11, -13, -17, and -35 leaf tissue 24 hours after wounding. One Unit (U)=0.001 OD per minute. Specific activity is defined as one U per mg protein.

FIG. 16 is an alignment of grape PPO1, PPO2, PPO3, PPO4, and PPO5 proteins. The tyrosinase domain is indicated with boxes surrounding it.

### DETAILED DESCRIPTION

The present disclosure is directed to grape cells, grape plants, grape plant parts, grape seeds, and grape fruits having one or more polyphenol oxidase (PPO) gene mutations. Grape plants having one or more polyphenol oxidase (PPO) gene mutations according to the present disclosure are expected to have traits such as reduced browning of post-harvest fruit, increased shelf life of post-harvest fruit, and fruit with increased nutritional value compared to wild type grape plants (i.e., grape plants that do not have one or more polyphenol oxidase (PPO) gene mutations. The present disclosure also pertains to methods of obtaining grape cells, grape plants, grape plant parts, grape seeds, and grape fruits having one or more polyphenol oxidase (PPO) gene mutations.

Unless otherwise indicated, the definitions and embodiments described in this and other sections are intended to be applicable to all embodiments and aspects of the present disclosure herein described for which they are suitable as would be understood by a person of ordinary skill in the art.

Singular forms “a”, “an”, and “the” include plural references unless the context clearly dictates otherwise. Thus, for example, a reference to “a method” includes one or more methods, and/or steps of the type described herein and/or which will become apparent to a person of ordinary skill in the art upon reading this disclosure. In another example, reference to “a cell” includes both a single cell and a plurality of cells.

The term “about” includes being within a statistically meaningful range of a value. Such a range can be within an order of magnitude, such as within 10% or within 5% of a given value or range.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers, and/or steps. The foregoing also applies to words having similar meanings such as the terms, “including”, “involving”, “having”, and their derivatives. The term “consisting of” and its derivatives, as used herein, are intended to be closed terms that specify the presence of the stated features, elements, components, groups, integers, and/or steps, but exclude the presence of other unstated features, elements, components, groups, integers, and/or steps.

The terms “nucleic acid”, “nucleotide”, or “polynucleotide” sequence are used interchangeably, and refer to a polymeric compound comprised of covalently linked subunits called nucleotides. Nucleic acids include polyribonucleic acid (“RNA”) and polydeoxyribonucleic acid (“DNA”), both of which may be single-stranded or double-stranded. DNA includes, but is not limited to, cDNA, genomic DNA, plasmid DNA, synthetic DNA, and semi-synthetic DNA. DNA may be linear, circular, or supercoiled.

As used herein, a “gene” refers to an assembly of nucleotides that typically encodes a polypeptide or protein and includes cDNA and genomic DNA nucleic acids. “Gene” also refers to a nucleic acid fragment that expresses a specific functional RNA, protein, or polypeptide, optionally including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source but arranged in a manner different than that found in nature. A chimeric gene may comprise coding sequences derived from different sources and/or regulatory sequences derived from different sources. “Endogenous gene” refers to a native gene in its natural location in the genome of an organism. “Heterologous” or “exogenous” DNA refers to DNA not naturally located in the cell, or in a chromosomal site of the cell. Heterologous genes can comprise native genes inserted into a non-native organism, or chimeric genes. A “transgene” is a gene that has been introduced into the genome by a transformation or transfection procedure.

“Transfection” and “transfected” are used inter-changeably herein for any kind of introduction of a material, including a nucleic acid (DNA/RNA), ribonucleoprotein (“RNP”) complex, amino acid, chemical, metabolite, nanoparticle, microparticle, combinations, and the like into at least one cell of interest by any kind of physical (e.g., bombardment) or chemical way of introducing the relevant at least one material.

A “reference sequence” means a nucleic acid or amino acid used as a comparator for another nucleic acid or amino acid, respectively, when determining sequence identity. A reference sequence can be a wild-type sequence.

“Sequence identity,” “percent identity,” or “% identical” refers to the exactness of a match between a reference sequence and a sequence being compared to it when optimally aligned. For example, sequence alignments and percent identity calculations may be determined using a variety of comparison methods designed to detect homologous sequences including, but not limited to, the Multalin program (Corpet, “Multiple Sequence Alignment with Hierarchical Clustering,” Nucleic Acids Res. 16:10881-90 (1988), which is hereby incorporated by reference in its entirety) or the Megalign® program of the LASERGENE® bioinformatics computing suite (DNASTAR® Inc., Madison, Wis.). Sequences may also be aligned using algorithms known in the art including, but not limited to, CLUSTAL V algorithm or the BLASTN or BLAST 2 sequence programs.

### PPO Genes in Grape

One aspect of the present disclosure relates to a grape plant cell comprising one or more loss-of-function mutations of a nucleic acid sequence encoding a polyphenol oxidase (PPO) gene selected from one or more of PPO1, PPO2, PPO3, PPO4, and PPO5, wherein the grape plant cell has reduced polyphenol oxidase gene expression or activity compared to a wild-type grape plant cell.

This aspect of the present disclosure can be carried out with any of the embodiments disclosed herein.

In some embodiments, the present disclosure relates to modifying a plant or a plant cell to reduce or eliminate the expression and/or activity of a Vitis vinifera polyphenol oxidase (PPO) gene or protein.

The nucleotide sequences, the coding sequences, and the amino acid sequences of the grape PPO genes described herein as PPO1, PPO2, PPO3, PPO4, and PPO5, and the nucleotide sequence of pseudogene PPO6, are set forth in Table 5 (infra).

The nucleotide sequence for the grape (Vitis vinifera) PPO1 gene (SEQ ID NO:1; GenBank Accession No. NM_001281116.1, which is hereby incorporated by reference in its entirety), the coding nucleotide sequence for PPO1 (SEQ ID NO:2), and the amino acid sequence for the PPO1 protein (SEQ ID NO:3) are set forth in Table 5 (infra).

The nucleotide sequence for the grape (Vitis vinifera) PPO2 gene (SEQ ID NO:4; GenBank Accession No. XM_010648796.2, which is hereby incorporated by reference in its entirety), the coding nucleotide sequence for PPO2 (SEQ ID NO:5), and the amino acid sequence for the PPO2 protein (SEQ ID NO:6) are set forth in Table 5 (infra).

The nucleotide sequence for the grape (Vitis vinifera) PPO3 gene (SEQ ID NO:7; GenBank Accession No. XM_019218532.1, which is hereby incorporated by reference in its entirety), the coding nucleotide sequence for PPO3 (SEQ ID NO:8), and the amino acid sequence for the PPO3 protein (SEQ ID NO:9) are set forth in Table 5 (infra).

The nucleotide sequence for the grape (Vitis vinifera) PPO4 gene (SEQ ID NO:10; GenBank Accession No. XM_010648798.2, which is hereby incorporated by reference in its entirety), the coding nucleotide sequence for PPO4 (SEQ ID NO:11), and the amino acid sequence for the PPO4 protein (SEQ ID NO:12) are set forth in Table 5 (infra).

The nucleotide sequence for grape (Vitis vinifera) PPO5 (SEQ ID NO:13; GenBank Accession No. XR_002029618.1, which is hereby incorporated by reference in its entirety), the coding nucleotide sequence for PPO5 (SEQ ID NO:14), and the amino acid sequence for the PPO5 protein (SEQ ID NO:15) are set forth in Table 5 (infra).

The nucleotide sequence for grape (Vitis vinifera) PPO6 (SEQ ID NO:16; NC_012009.3:17002090-17003833 Vitis vinifera cultivar PN40024 chromosome 3, 12X, whole genome shotgun sequence, which is hereby incorporated by reference in its entirety), is set forth in Table 5 (infra).

The mutations (and methods of generating mutations described infra) described herein are applicable to PPO1 genes from any variety of grape with a nucleic acid sequence that is at least 80% identical to SEQ ID NO:1. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO:1.

The mutations (and methods of generating mutations described infra) described herein are applicable to PPO2 genes from any variety of grape with a nucleic acid sequence having at least 80% sequence identity to SEQ ID NO:4. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO:4.

The mutations (and methods of generating mutations described infra) described herein are applicable to PPO3 genes from any variety of grape with a nucleic acid sequence having at least 80% sequence identity to SEQ ID NO:7. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO:7.

The mutations (and methods of generating mutations described infra) described herein are applicable to PPO4 genes from any variety of grape with a nucleic acid sequence having at least 80% sequence identity to SEQ ID NO:10. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO:10.

The mutations (and methods of generating mutations described infra) described herein are applicable to PPO5 genes from any variety of grape with a nucleic acid sequence having at least 80% sequence identity to SEQ ID NO:13. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO:13.

The mutations (and methods of generating mutations described infra) described herein are applicable to PPO1 genes from any variety of grape with a coding sequence having at least 80% sequence identity to SEQ ID NO:2. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO:2.

The mutations (and methods of generating mutations described infra) described herein are applicable to PPO2 genes from any variety of grape with a coding sequence having at least 80% sequence identity to SEQ ID NO:5. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO:5.

The mutations (and methods of generating mutations described infra) described herein are applicable to PPO3 genes from any variety of grape with a coding sequence having at least 80% sequence identity to SEQ ID NO:8. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO:8.

The mutations (and methods of generating mutations described infra) described herein are applicable to PPO4 genes from any variety of grape with a coding sequence having at least 80% sequence identity to SEQ ID NO:11. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO:11.

The mutations (and methods of generating mutations described infra) described herein are applicable to PPO5 genes from any variety of grape with a coding sequence having at least 80% sequence identity to SEQ ID NO:14. Also encompassed are nucleic acid sequences having at least 80%, 81%, 82%, 83%, 84%, 85%, 86%, 87%, 88%, 89%, 90%, 91%, 92%, 93%, 94%, 95%, 96%, 97%, 98%, 99% sequence identity, or 100% sequence identity to the entire sequence of SEQ ID NO: 14.

The mutations (and methods of generating mutations described infra) described herein are applicable to any variety of grape, including those with a polyphenol oxidase PPO1 protein having an amino acid sequence having at least 80% sequence identity to SEQ ID NO:3. In some embodiments, the PPO1 protein has an amino acid sequence having at least 80%, 83%, 85%, 90%, 93%, 95%, 98%, 99%, or 100% sequence identity to the amino acid sequence of SEQ ID NO:3.

The mutations (and methods of generating mutations described infra) described herein are applicable to any variety of grape, including those with a polyphenol oxidase PPO2 protein having an amino acid sequence having at least 80% sequence identity to SEQ ID NO:6. In some embodiments, the PPO2 protein has an amino acid sequence having at least 80%, 83%, 85%, 90%, 93%, 95%, 98%, 99%, or 100% sequence identity to the amino acid sequence of SEQ ID NO:6.

The mutations (and methods of generating mutations described infra) described herein are applicable to any variety of grape, including those with a polyphenol oxidase PPO3 protein having an amino acid sequence having at least 80% sequence identity to SEQ ID NO:9. In some embodiments, the PPO3 protein has an amino acid sequence having at least 80%, 83%, 85%, 90%, 93%, 95%, 98%, 99%, or 100% sequence identity to the amino acid sequence of SEQ ID NO:9.

The mutations (and methods of generating mutations described infra) described herein are applicable to any variety of grape, including those with a polyphenol oxidase PPO4 protein having an amino acid sequence having at least 80% sequence identity to SEQ ID NO:12. In some embodiments, the PPO4 protein has an amino acid sequence having at least 80%, 83%, 85%, 90%, 93%, 95%, 98%, 99%, or 100% sequence identity to the amino acid sequence of SEQ ID NO:12.

The mutations (and methods of generating mutations described infra) described herein are applicable to any variety of grape, including those with a polyphenol oxidase PPO5 protein having an amino acid sequence having at least 80% sequence identity to SEQ ID NO:9. In some embodiments, the PPO5 protein has an amino acid sequence having at least 80%, 83%, 85%, 90%, 93%, 95%, 98%, 99%, or 100% sequence identity to the amino acid sequence of SEQ ID NO:15.

As described herein, one or more mutations introduced into a Vitis vinmfera polyphenol oxidase (PPO) gene may be present in one or more of a grape PPO gene or genes. In some embodiments, the one or more mutations in the PPO gene or genes are human-induced mutations. In some embodiments, the one or more mutations in the PPO gene or genes are loss-of-function mutations.

As used herein, the phrase “loss-of-function mutation” refers to a mutation that results in a gene or gene product no longer being able to perform its normal function, or no longer having its normal level of activity, in whole or in part, compared to a wild-type (un-mutated) counterpart. Loss-of-function mutations are also referred to as inactivating mutations that typically result in the gene product having less or no function, i.e., being partially or wholly inactivated. Loss-of-function mutations include insertions and deletions that interrupt or change the coding region of a gene, such as causing a premature stop codon or altering the splicing of a nucleotide sequence.

A loss-of-function mutation that introduces a premature stop codon in the coding sequence or eliminates or changes an amino acid residue that is essential for catalytic function of an enzyme is referred to herein as a “knockout” mutation. In some embodiments, the loss-of-function mutation encodes a knockout mutation. In some embodiments, the loss-of-function mutation is an insertion mutation. In some embodiments, the loss-of-function mutation is a deletion mutation. In some embodiments, the loss-of-function mutation is a combination of one or more insertion mutations. In some embodiments, the loss-of-function mutation is a combination of one or more deletion mutations. In some embodiments, the loss-of-function mutation is a combination of one or more insertion mutations and one or more deletion mutations. Loss-of-function mutations result from altering the reading frame of a nucleic acid sequence, regardless of whether it is an insertion, deletion, or a combination of an insertion and deletion. For example, an insertion or deletion of 1, 2, 4, 5, 7, 8, 10, 11, 13, 14, 16, 17, 19, 20, or more than 20 nucleotides would alter the reading frame, or alter it sufficiently to provide a loss-of-function. Insertions or deletions, or combinations thereof, totaling 3, 6, 9, 12, 15, or 18 nucleotides would not alter the reading frame, however in-frame insertions or deletions larger than 21 nucleotides are expected to provide a loss-of-function.

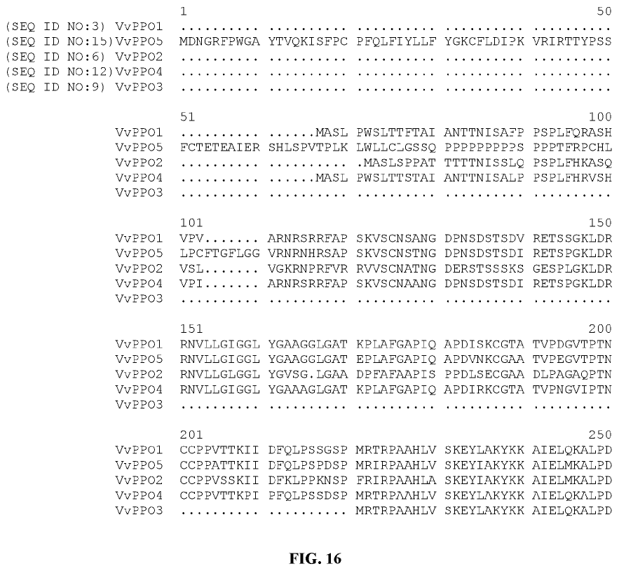

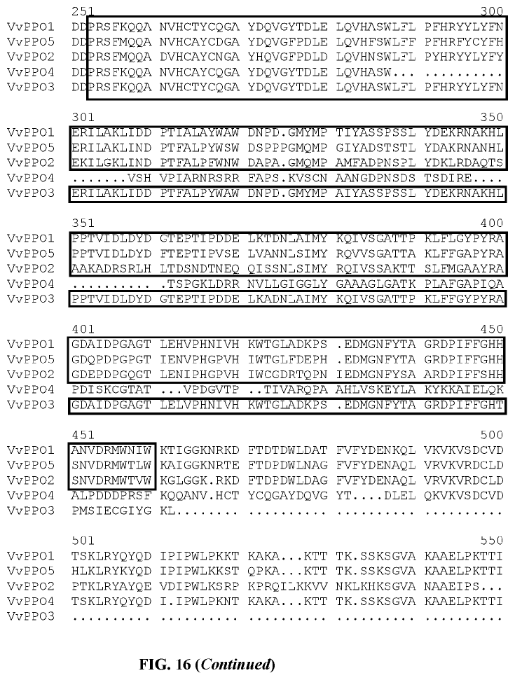

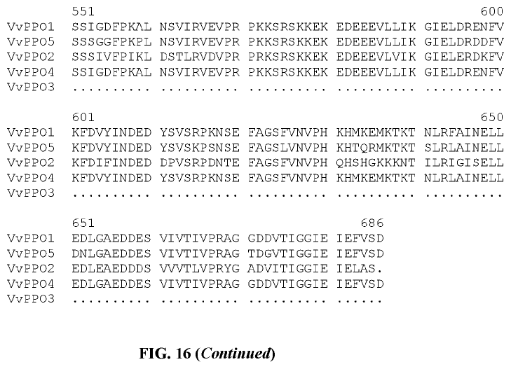

In some embodiments, the polypeptides or proteins according to this or any other embodiment described herein comprise one or more (e.g., 1, 2, 3, 4, 5, or more) amino acid insertions, deletions, or other modifications (e.g., substitution of one amino acid for another) compared to a wild-type sequence. In some embodiments, the loss-of-function mutation is a knockout mutation that disrupts the key structural domain of polyphenol oxidase, namely the copper binding tyrosinase domains. Altering the reading frame of the protein with an insertion or deletion (or combination thereof) that leads to a premature stop codon is one means of altering the tyrosinase domain. Other mutations that do not change the reading frame (such as a missense mutation or in-frame deletion) but eliminate or alter essential amino acids in the tyrosinase domain are also considered loss-of-function mutations. A box identifying the tyrosinase domain is shown in an alignment of grape PPO sequences in FIG. 16.

While specific mutations leading to loss of function of PPO genes are described herein, a person of ordinary skill in the art will appreciate, based on the discussion above, that that other mutations will lead to loss of function mutations, all of which are encompassed by the present disclosure.

In some embodiments, the same mutation in a PPO gene occurs in both chromosomal alleles of that PPO gene nucleic acid sequence. In other words, the mutation is a homozygous mutation. In other embodiments, the mutation in a PPO gene occurs in only one chromosomal allele of that PPO gene. In other words, the mutation is a heterozygous mutation. In yet another embodiment, two different mutations in the same PPO gene can occur in each chromosomal allele of the same PPO gene such that both alleles comprise different mutations in the PPO gene. In some embodiments, the one or more loss-of-function mutations are in at least one chromosomal allele of the nucleic acid sequence. In some embodiments, the one or more loss-of-function mutations are in both chromosomal alleles of the nucleic acid sequence.

In some embodiments, grape cells of the present disclosure comprise one or more loss-of-function mutations in the nucleic acid sequence encoding the PPO1 gene. In some embodiments, the one or more loss-of-function mutations are in the PPO1 nucleotide sequence of SEQ ID NO:2, or a nucleotide sequence that has at least 95% sequence identity to SEQ ID NO:2. In some embodiments, the grape cell comprises one or more loss-of-function mutations in the nucleic acid sequence encoding the PPO2 gene. In some embodiments, the one or more loss-of-function mutations are in the PPO2 nucleotide sequence of SEQ ID NO: 5, or a nucleotide sequence that has at least 95% sequence identity to SEQ ID NO:5. In some embodiments, the grape cell comprises one or more loss-of-function mutations in the nucleic acid sequence encoding the PPO3 gene. In some embodiments, the one or more loss-of-function mutation are in the PPO3 nucleotide sequence of SEQ ID NO:8, or a nucleotide sequence that has at least 95% sequence identity to the nucleotide sequence of SEQ ID NO:8. In some embodiments, the grape cell comprises one or more loss-of-function mutations in the nucleic acid sequence encoding the PPO4 gene. In some embodiments, the one or more loss-of-function mutations are in the PPO4 nucleotide sequence of SEQ ID NO: 11, or a nucleotide sequence that has at least 95% sequence identity to the nucleotide sequence of SEQ ID NO:11. In some embodiments, the grape cell comprises one or more loss-of-function mutations in the nucleic acid sequence encoding the PPO5 gene. In some embodiments, the one or more loss-of-function mutations are in the PPO5 nucleotide sequence of SEQ ID NO:14, or a nucleotide sequence that has at least 95% sequence identity to the nucleotide sequence of SEQ ID NO:14.

In some embodiments, the grape cell comprises a loss-of-function mutation in two of the PPO1, PPO2, PPO3, PPO4, and PPO5 genes. In some embodiments, the grape cell comprises a loss-of-function mutation in three of the PPO1, PPO2, PPO3, PPO4, and PPO5 genes. In some embodiments, the grape cell comprises a loss-of-function mutation in four of the PPO1, PPO2, PPO3, PPO4, and PPO5 genes. In some embodiments, the grape cell comprises a loss-of-function mutation in each of the PPO1, PPO2, PPO3, PPO4, and PPO5 genes.

In some embodiments, the grape cell comprises a first loss-of-function mutation in the PPO2 gene and a second loss-of-function mutation in one or more of PPO1, PPO3, PPO4, and PPO5 genes. In some embodiments, the grape cell comprises a first loss-of-function mutation in the PPO2 gene and a second loss-of-function mutation in the PPO1 gene. In some embodiments, the grape cell comprises a first loss-of-function mutation in the PPO2 gene, a second loss-of-function mutation in the PPO1 gene, and a third loss-of-function mutation in the PPO3 gene. In some embodiments, the grape cell further comprises one or more additional loss-of-function mutations in the PPO4 and/or PPO5 genes.

In some embodiments, the grape cell comprises a loss-of-function mutation in both chromosomal alleles of the PPO2 gene and a loss-of-function mutation in both chromosomal alleles of one or more of the PPO1, PPO3, PPO4, and PPO5 genes.

In some embodiments, the grape cell comprises a loss-of-function mutation in both chromosomal alleles of the PPO2 gene and a loss-of-function mutation in both chromosomal alleles of the PPO1 and PPO3 genes.

In some embodiments, the grape cell comprises a loss-of-function mutation in both chromosomal alleles of the PPO2 gene and a loss-of-function mutation in both chromosomal alleles of the PPO1, PPO3, and PPO5 genes.

A grape cell of the present disclosure may contain combinations of genotypes of different PPO genes. For example, a grape cell may have homozygous loss-of-function mutations of some PPO genes, heterozygous loss-of-function mutations of some PPO genes, different loss-of-function mutations in either chromosome of the same gene of some PPO genes, or wild-type PPO genes of some PPO genes, or any combination thereof.

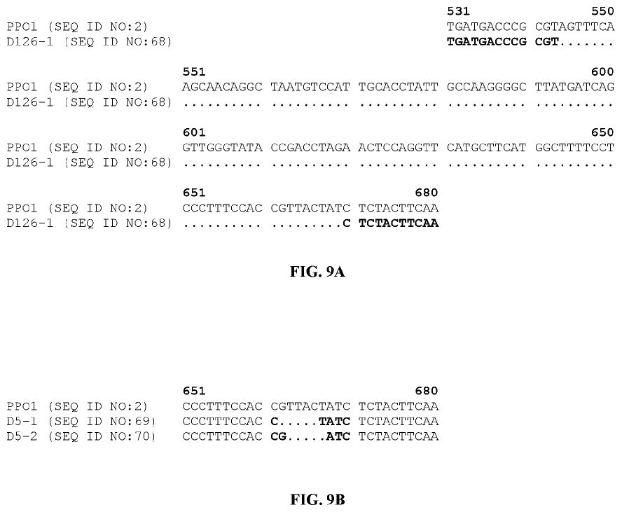

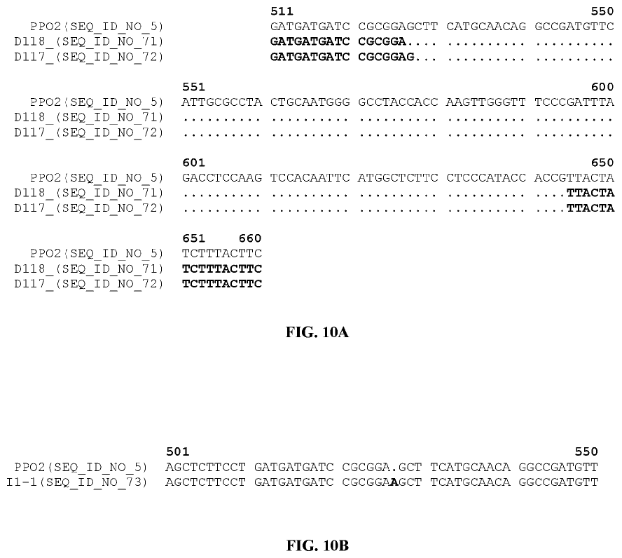

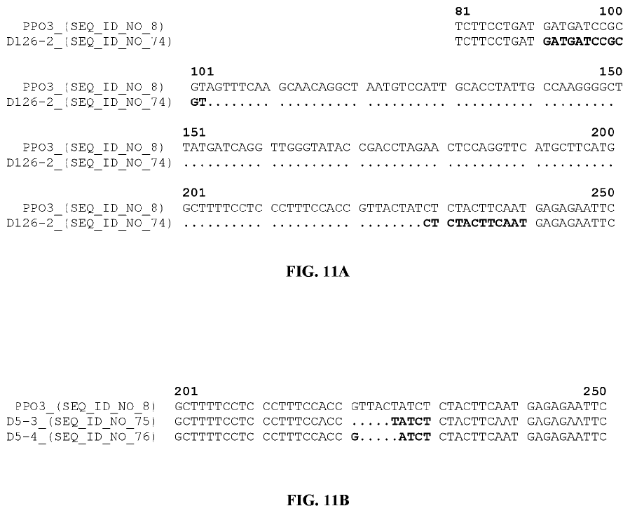

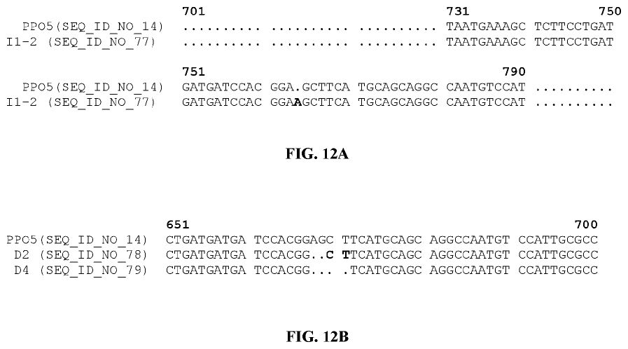

Exemplary genome edited mutations are shown in Table 4 (infra) and FIGS. 9A-B, FIGS. 10A-B, FIGS. 11A-B, and FIGS. 12A-B. In some embodiments, the one or more loss-of-function mutations in PPO1 are selected from SEQ ID NOs:68-70. In some embodiments, the one or more loss-of-function mutations in PPO2 are selected from SEQ ID NOs:71-73. In some embodiments, the one or more loss-of-function mutations in PPO3 are selected from SEQ ID NOs:74-76. In some embodiments, the one or more loss-of-function mutations in PPO5 are selected from SEQ ID NOs:77-79.

In addition to grape cells, the present disclosure provides grape plants, plant parts, seeds, and fruit, as well as beverages (e.g., wine and grape juice) and food products made from the grape cells, plants, plant parts, and/or fruit that have one or more loss-of-function mutations in one or more polyphenol oxidase (PPO) genes.

In some embodiments, a grape plant comprises the grape plant cell described herein. In some embodiments, a grape fruit comprises the grape plant cell described herein. In some embodiments, a grape plant, plant part, seed, or fruit propagated from the grape cell, grape plant or grape fruit described herein, comprises a loss-of-function mutation in one or more polyphenol oxidase (PPO) genes.

### Methods of Modifying PPO Genes in Grape

Modifying a gene in a grape cell, plant, plant part, seed, and/or fruit, so that the cell, plant, plant part, seed, and/or fruit possesses a gene with a loss-of-function mutation, may be done by any method known in the art. That is, any method known in the art for introducing mutations into a cell in a gene sequence may be used to achieve the grape plant cell of the present disclosure. Such methods should be effective for introducing mutations into one or more of the PPO1, PPO2, PPO3, PPO4, and PPO5 genes of grape, as well as grape cells, plants, plant parts, seeds, and fruits with mutations in two or more of the PPO1, PPO2, PPO3, PPO4, and PPO5 genes, and, e.g., any combination of any one PPO gene with any of the other PPO genes, such as PPO2 mutations with one or more mutations in PPO1, PPO3, PPO4, and PPO5 as described herein.

The terms “grape cell” or “grape plant cell” are used interchangeably, and include cells, protoplasts, cell tissue cultures from which grape plants can be regenerated, calli, clumps, and cells that are intact in grape or parts of grape including, but not limited to seeds, leaves, stems, roots, vegetative buds, floral buds, meristems, embryos, hypocotyls, cotyledons, endosperm, sepals, petals, pistils, carpels, stamens, anthers, microspores, pollen, pollen tubes, ovules, nucellar tissue, ovaries, and other grape tissue or cells. In some embodiments, the grape cell is a protoplast.

In some embodiments, the grape cell, plant, plant part, seed, or fruit is modified by genome editing. In some embodiments, the grape cell is a regenerable grape cell. In some embodiments, a grape plant comprises the grape cell. In some embodiments, a grape fruit comprises the grape cell. In some embodiments, a grape plant, plant part, seed, or fruit is propagated (sexually or asexually) from a grape plant, plant part, seed, or fruit of any of the embodiments of the present disclosure.

There are a variety of grapes grown throughout the world that are applicable to the present disclosure and, indeed, any type or variety of grape is contemplated by the present disclosure. Suitable types of grapes include green, white, black, and red grapes.

Suitable varieties of grapes include, without limitation, red and white Vinifera varietals and hybrid grapes. Non-limiting examples of white grapes include Arbane, Chardonnay, Chenin Blanc, Gewürztraminer, Grenache Blanc, Gruner Veltliner, Marsanne, Muscat, Pinot Blanc, Pinot Gris, Reisling, Roussanne, Sauvignon Blanc, Sémillion, and Viognier.

Non-limiting examples of red grapes include Gamay, Pinot Noir, Tempranillo, Sangiovese, Merlot, Zinfandel, Cabernet Sauvignon, Nebbiolo, Syrah, Petit Sirah, Malbec, Shiraz, Garnacha, Páis Grenache, Mourvédre, Picpoul, Terret, Counoise, Muscardin, Rioja, Barbera, Cabernet Franc, Pixie, Carignane, Charbono, Tinto Madeira, Vaccarēse, Picordin, Cinsault, Clairette, Roussanne, Bourboulenc, Dolcetto, and Barbera. Most all of these and many other grape varieties and wines are discussed in Zraly, “Windows of the World, Complete Wine Course,” Millennium Edition, New York, N.Y., (Sterling Publishing) 2000, which is hereby incorporated by reference in its entirety.

In some embodiments, the grape plant cell, plant, plant part, seed, or fruit is selected from the variety Arbane, Chardonnay, Chenin Blanc, Gewürztraminer, Grenache, Grenache Blanc, Gruner Veltliner, Gamay, Marsanne, Muscat, Pinot Blanc, Pinot Gris, Reisling, Roussanne, Sauvignon Blanc, Sémillion, Viognier, Pinot Noir, Tempranillo, Sangiovese, Merlot, Zinfandel, Cabernet Sauvignon, Nebbiolo, Syrah, Petit Sirah, Malbec, Shiraz, Garnacha, Páis Grenache, Mourvédre, Muscat, Picpoul, Petit Meslier, Pixie, Terret, Counoise, Muscardin, Rioja, Rose, Barbera, Cabernet Franc, Carignane, Charbono, Tinto Madeira, Vaccarēse, Picordin, Cinsault, Clairette, Roussanne, Bourboulenc, Dolcetto, Barbera, and hybrids thereof.

In some embodiments, a beverage or a food product is made from the grape fruit as described herein. In some embodiments, a beverage is made from the grape fruit of a green, white, black, or red grape variety, or combinations thereof. In some embodiments, a beverage is made from the grape fruit of the variety Arbane, Chardonnay, Chenin Blanc, Gewürztraminer, Grenache, Grenache Blanc, Gruner Veltliner, Gamay, Marsanne, Muscat, Pinot Blanc, Pinot Gris, Reisling, Roussanne, Sauvignon Blanc, Sémillion, Viognier, Pinot Noir, Tempranillo, Sangiovese, Merlot, Zinfandel, Cabernet Sauvignon, Nebbiolo, Syrah, Petit Sirah, Malbec, Shiraz, Garnacha, Páis Grenache, Mourvédre, Muscat, Picpoul, Petit Meslier, Pixie, Terret, Counoise, Muscardin, Rioja, Rose, Barbera, Cabernet Franc, Carignane, Charbono, Tinto Madeira, Vaccarēse, Picordin, Cinsault, Clairette, Roussanne, Bourboulenc, Dolcetto, Barbera, and combinations thereof.

In some embodiments, a mutation may be induced by treatment with a mutagenic agent. Any suitable mutagenic agent can be used for embodiments of the present disclosure. For example, mutagens creating point mutations, deletions, insertions, rearrangements, transversions, transitions, or any combination thereof may be used. Suitable radiation mutagens include, without limitation, ultraviolet light, x-rays, gamma rays, and fast neutrons. Suitable chemical mutagens include, but are not limited to, ethyl methanesulfonate (“EMS”), methylmethane sulfonate (“MMS”), N-ethyl-N-nitrosourea (“ENU”), triethylmelamine (“TEM”), N-methyl-N-nitrosourea (“MNU”), procarbazine, chlorambucil, cyclophosphamide, diethyl sulfate, acrylamide monomer, melphalan, nitrogen mustard, vincristine, dimethylnitrosamine, N-methyl-N′-nitro-nitrosoguanidine 25 (“MNNG”), nitrosoguanidine, 2-aminopurine, 7, 12 dimethyl-benz(a)anthracene (“DMBA”), ethylene oxide, hexamethylphosphoramide, bisulfan, diepoxyalkanes (diepoxyoctane (“DEO”), diepoxybutane (“DEB”), 2-methoxy-6-chloro-9[3-(ethyl-2-chloro-ethyl) aminopropylamino] acridine dihydrochloride (“ICR-170”), sodium azide, formaldehyde, or combinations thereof.

A mutation may be detected using a method such as “TILLING” or “Targeting Induced Local Lesions in Genomes” which is a general reverse genetic method providing an allelic series of induced mutation by random chemical or physical mutagenesis, that can be used to identify mutations in a gene or region of interest. In a common use of the TILLING methodology, plant material, such as seeds, are subjected to chemical mutagenesis, which creates a series of mutations within the genomes of the seeds' cells. The mutagenized seeds are grown into adult M1 plants and self-pollinated. DNA samples from the resulting M2 plants are pooled and are then screened for mutations in a gene of interest. Once a mutation is identified in a gene of interest, the seeds of the M2 plant carrying that mutation are grown into adult M3 plants and screened for the phenotypic characteristics associated with the gene of interest. See for example, Colbert et al., “High-Throughput Screening for Induced Point Mutations,” Plant Physiology 126:480-484 (2001) and Krasileva et al., “Uncovering Hidden Variation in Polyploid Wheat,” Proc. Nat. Acad. Sci. 114-E913-E921 (2017), each of which is hereby incorporated by reference in its entirety.

In some embodiments, a PPO gene mutation is induced in a plant cell of the present disclosure by genome editing. Genome editing is a type of genetic engineering in which DNA is inserted, replaced, or deleted, or any combination thereof, from a genome using artificially engineered nucleases, or “molecular scissors.” The nucleases typically create double-stranded breaks (“DSBs”) at desired locations in the genome and harness the cell's endogenous mechanisms to repair the induced break by processes of homology dependent repair (“HDR”) or nonhomologous end-joining (“NHEJ”). Any method of genome editing may be used in the embodiments of the present disclosure.

CRISPR/Cas type RNA-guided endonucleases provide an efficient system for inducing genetic modifications in genomes of many organisms. Non-limiting examples of genome editing nucleases include Cas1, Cas1B, Cas2, Cas3, Cas4, Cas5, Cas6, Cas7, Cas8, Cas9 (also known as Csn1 and Csx12), Cas10, Cas12a (Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, Mad7, SynNuc1, or homologs, modified versions, and endonuclease inactive versions thereof. An example of a fusion protein to Cas9 is a cytidine deaminase-Cas9 fusion protein used in cytidine base editing to mutate nucleotides in target genes without generating double-strand breaks as described in Komor et al., “Programmable Editing of a Target Base in Genomic DNA without Double-Stranded DNA Cleavage,” Nature 533:420-424 (2016), which is hereby incorporated by reference in its entirety. The use of CRISPR guide RNA in conjunction with CRISPR/Cas technology to target RNA is also described in Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339:819-23 (2013); and Gaj et al., “ZFN, TALEN, and CRISPR/Cas-based Methods for Genome Engineering,” Cell 31:397-405 (2013), each of which is hereby incorporated by reference in its entirety.

There are typically two distinct components to a CRISPR system, a guide RNA (“gRNA”) and a genome editing endonuclease. The gRNA uses a CRISPR RNA (“crRNA”) comprising a DNA targeting segment that can be engineered to contain a complementary stretch of nucleotide sequence (e.g., at least 10 nucleotides) to target a DNA site for binding and subsequent modification by CRISPR genome editing nuclease. The length of a crRNA may range from about 15 nucleotides to about 60 nucleotides. The crRNA can be chemically synthesized and can also be engineered to include a ribonucleotide analog or a modified form thereof, or an analog of a modified form, or non-natural nucleosides. Exemplary portions of gRNA sequences that target grape PPO genes are provided below, without limitation. These gRNAs target sequences in the tyrosinase domain of the grape PPO genes, which is shown in FIG. 16. Other gRNAs are contemplated. In some embodiments, gRNAs are directed to sequences shared in common between multiple PPO genes in order to target and edit more than one PPO gene at a time resulting in mutations in more than one PPO gene. In some embodiments, gRNAs can be directed to a PPO gene sequence that is not shared with other PPO genes resulting in mutations in only one PPO gene. In some embodiments, the gRNAs comprise one or more of SEQ ID NOs:17-24.

(SEQ ID NO: 17)
GCCUGUUGCAUGAAGCUCCG
(SEQ ID NO: 18)
UCAUUGCGCCUACUGCAAUG
(SEQ ID NO: 19)
GAUCAUUAAUCAACUUUCCC
(SEQ ID NO: 20)
GGCAAAGGCGAAUGGAUCAG
(SEQ ID NO: 21)
AUGAACCUGGAGUUCUAGGU
(SEQ ID NO: 22)
UGAAGUAGAGAUAGUAACGG
(SEQ ID NO: 23)
GGGGAACUAGCAUAGAUGGU
(SEQ ID NO: 24)
GUGAUCGAUCUCGACUACGA

Depending on the genome editing nuclease used, the gRNA can also comprise a trans-activating crRNA (“tracrRNA”). Such is the case with Cas9, for example. The tracrRNA is a small RNA sequence that forms a binding handle used by the CRISPR protein. The tracrRNA can be chemically synthesized and can also be engineered to include a ribonucleotide analog or a modified form thereof, or an analog of a modified form, or non-natural nucleosides.

The term gRNA also includes single guide RNAs (“sgRNA”), which combine the targeting specificity of the crRNA with the scaffolding properties of the tracrRNA. sgRNAs can be synthesized or expressed as a continuous RNA transcript. Alternatively, two-part gRNAs can be assembled from two separate RNAs by combining a crRNA-containing RNA with a tracrRNA. In the sgRNA or the two-part gRNA, crRNA and tracrRNA are present either in their native form, or a modified form. Either type of gRNA may be about 60 nucleotides to about 120 nucleotides long. These gRNAs can be chemically synthesized and can also be engineered to include a ribonucleotide analog or a modified form thereof, or an analog of a modified form, or non-natural nucleosides.

When the gRNA and the gene editing endonuclease are introduced into the cell, the genomic target sequence can be modified or permanently disrupted to create a loss-of-function mutation(s). A complex of a genome editing nuclease with a gRNA is called a ribonucleotide particle or ribonucleoprotein (RNP) complex. The RNP complex is recruited to the target sequence by the base-pairing between the gRNA sequence, which has a region of complementarity to the target sequence in the genomic DNA. In some embodiments, the target sequence is a sequence selected from one or more of SEQ ID NOs:1, 2, 4, 5, 7, 8, 10, 11, 13, 14, and 16 or portions thereof.

For successful binding of Cas9, the genomic target sequence must also contain the correct Protospacer Adjacent Motif (“PAM”) sequence immediately following the target sequence. The binding of the RNP complex localizes the genome editing nuclease to the genomic target sequence so that the genome editing nuclease can cut both strands of DNA causing a DSB. Cas9 generates DSBs through the combined activity of two nuclease domains, RuvC and HNH. Cas9 will cut 3-4 nucleotides upstream of the PAM sequence. CRISPR specificity can be controlled by level of homology and binding strength of the specific gRNA for a given gene target, or by modification of the Cas endonuclease itself. For example, a D10A mutant of the RuvC domain, retains only the HNH domain and generates a DNA nick rather than a DSB.

A software tool can be used to optimize the choice of gRNA within a target sequence, and to minimize total off-target activity across the rest of the genome. The cleavage efficiency at each off-target sequence can be estimated, e.g., using an experimentally-derived weighting scheme. Each possible gRNA is then ranked according to its total predicted off-target cleavage; the top-ranked gRNAs represent those that are likely to have the greatest on-target and the least off-target cleavage. An exemplary software tool to use for estimating gRNA cleavage efficiency is Geneious software (Geneious, San Diego, CA).

Other nucleases can also be used for genome editing. ZFNs are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. ZFNs include an engineered zinc finger DNA-binding domain fused to the cleavage domain of the FokI restriction endonuclease. ZFNs can be used to induce double-stranded breaks (DSBs) in specific DNA sequences.

TALEN is a sequence-specific endonuclease that includes a transcription activator-like effector (“TALE”) and a FokI endonuclease. The transcription activator-like effector is a DNA binding protein that has a highly conserved central region with tandem repeat units of 34 amino acids. The base preference for each repeat unit is determined by two amino acid residues called the repeat-variable di-residue, which recognizes one specific nucleotide in the target DNA. Arrays of DNA-binding repeat units can be customized for targeting specific DNA sequences. As with ZFNs, dimerization of two TALENs on targeted specific sequences in a genome results in FokI-dependent introduction of double stranded breaks, stimulating homology directed repair (“HDR”) and non-homologous end joining (NHEJ) repair mechanism.

Meganucleases with re-engineered homing nucleases can also be used to effect genome modification in plants in the methods described herein. Meganucleases are endodeoxyribonucleases characterized by a large recognition site (double-stranded DNA sequences of 12 to 40 base pairs). This site generally occurs only once in any given genome. For example, the 18-base pair sequence recognized by the I-Scel meganuclease would on average require a genome twenty times the size of the human genome to be found once by chance. Meganucleases are considered to be the most specific naturally occurring restriction enzymes. Among meganucleases, the LAGLIDADG family of homing endonucleases has become a valuable tool for the study of genomes and genome engineering over the past fifteen years. By modifying their recognition sequence through protein engineering, the targeted sequence can be changed.

Grape with Reduced PPO Gene Expression or Protein Levels

Additional embodiments of the present disclosure are directed to grape cells, plants, plant parts, seeds, and fruits with reduced PPO expression. The “expression” of a PPO gene refers to the transcription of a PPO gene. PPO gene expression levels may be measured by any means known in the art such as, without limitation, digital PCR, qRT-PCR (quantitative real time PCR), semi-quantitative PCR, RNA-seq, and Northern blot analysis.

In some embodiments, the expression of PPO genes are reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the expression of PPO genes in a wild-type grape cell, plant, plant part, seed, or fruit. In some embodiments, the expression of PPO genes is undetectable.

In some embodiments, the expression of a PPO1 gene is reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the expression of a PPO1 gene in a wild-type grape cell, plant, plant part, seed, or fruit. In some embodiments, the expression of a PPO1 gene is undetectable.

In some embodiments, the expression of a PPO2 gene is reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the expression of a PPO2 gene in a wild-type grape cell, plant, plant part, seed, or fruit. In some embodiments, the expression of a PPO2 gene is undetectable.

In some embodiments, the expression of any one or more of a PPO3, PPO4, and PPO5 gene is reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the expression of a PPO3, PPO4, and PPO5 gene in a wild-type grape cell, plant, plant part, seed, or fruit. In some embodiments, the expression of any one or more of a PPO3, PPO4, and PPO5 gene is undetectable.

The “amount” or “level” of a protein refers to the abundance of a particular protein, for example PPO2, which may be measured by any means known in the art such as, without limitation, Western blot analysis, ELISA, other forms of immunological detection, or mass spectrometry.

In some embodiments, the amount of total PPO protein is reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the amount of total PPO protein in a wild-type grape cell, plant, plant part, seed, or fruit. In some embodiments, the amount of total PPO protein is undetectable.

In some embodiments, the amount of a PPO1 protein is reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the amount of a PPO1 protein in a wild-type grape cell, plant, plant part, seed, or fruit. In some embodiments, the amount of a PPO1 protein is undetectable.

In some embodiments, the amount of a PPO2 protein is reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the amount of a PPO2 protein in a wild-type grape cell, plant, plant part, seed, or fruit. In some embodiments, the amount of a PPO2 protein is undetectable.

In some embodiments, the amount of a PPO3 protein is reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the amount of a PPO3 protein in a wild-type grape cell, plant, plant part, seed, or fruit. In some embodiments, the amount of a PPO3 protein is undetectable.

In some embodiments, the amount of PPO4 protein is reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the amount of the PPO4 protein in a wild-type grape cell, plant, plant part, seed, or fruit. In some embodiments, the amount of any one or more of a PPO4 protein is undetectable.

In some embodiments, the amount of a PPO5 protein is reduced by 100%, 95%, 90%, 85%, 80%, 75%, 70%, 65%, 60%, 55%, 50%, 45%, 40%, 35%, 30%, 25%, 20%, 15%, 10%, or 5%, or any number or range therein, in comparison to the amount of a PPO5 protein in a wild-type grape cell, plant, plant part, seed, or fruit. In some embodiments, the amount of a PPO5 protein is undetectable.

Grapes with Reduced PPO Activity

Additional aspects of the present disclosure are directed to grape cells, plants, plant parts, seeds, and fruits with reduced PPO activity. PPO protein “activity” or “PPO activity” refers to the enzymatic activity of the PPO protein(s). PPO protein activity may be measured biochemically by methods known in the art including, but not limited to, the detection of products formed by the enzyme in the presence of any number of substrates, for example, catechol and caffeic acid and/or increased levels of polyphenolic compounds as compared to a control (wild-type). In some embodiments, PPO protein activity is measured functionally, for example, by assessing its effects on phenotypic traits of a grape cell, plant, plant part, seed, or fruit, such as fruit or leaf browning when cut or bruised, and/or increased post-harvest shelf life as compared to a control (wild-type).

In some embodiments, PPO activity is reduced in grape plants of the present disclosure such that grapes, seeds, and plant parts harvested from the grape plants described herein (i.e., comprising one or more loss-of-function mutations of a nucleic acid sequence encoding a polyphenol oxidase (PPO) gene selected from one or more of PPO1, PPO2, PPO3, PPO4, and PPO5) demonstrate reduced post-harvest browning, increased post-harvest shelf life, and/or increased polyphenolics when compared to fruit, seeds, and plant parts of wild-type grape plants, seeds, and plant parts. In some embodiments, food products and beverages made from or comprising grapes, seeds, and/or plant parts from the grape plants described herein possess characteristics such as reduced browning, increased shelf life, and increased nutrition (e.g., increased polyphenolics) compared to a food or beverage products made from or comprising grapes, seeds, and/or plant parts from wild-type grape plants.

In some embodiments, the grape cell, plant, plant part, seed, or fruit of the present disclosure has total PPO activity that is reduced by 100%, 95%, 90%, 85%, 80%, 75%, 70%, 65%, 60%, 55%, 50%, 45%, 40%, 35%, 30%, 25%, 20%, 15%, 10%, 5%, or any number or range therein, of the activity of total PPO in wild-type grape cells, plants, plant parts, seeds, or fruits. In some embodiments, the grape cell, plant, plant part, seed, or fruit has undetectable total PPO activity. In some embodiments, the polyphenol oxidase activity is reduced by at least 10% compared to polyphenol oxidase activity of a wild-type grape plant cell. In some embodiments, the polyphenol oxidase activity is reduced by at least 20% compared to polyphenol oxidase activity of a wild-type grape plant cell. In some embodiments, the polyphenol oxidase activity is reduced by at least 30% compared to polyphenol oxidase activity of a wild-type grape plant cell. In some embodiments, the polyphenol oxidase activity is reduced by at least 40% compared to polyphenol oxidase activity of a wild-type grape plant cell. In some embodiments, the polyphenol oxidase activity is reduced by at least 50% or more compared to polyphenol oxidase activity of a wild-type grape plant cell. In some embodiments, the polyphenol oxidase activity is reduced by at least 60% or more compared to polyphenol oxidase activity of a wild-type grape plant cell. In some embodiments, the polyphenol oxidase activity is reduced by at least 70% or more compared to polyphenol oxidase activity of a wild-type grape plant cell. In some embodiments, the polyphenol oxidase activity is reduced by at least 80% or more compared to polyphenol oxidase activity of a wild-type grape plant cell. In some embodiments, the polyphenol oxidase activity is reduced by at least 90% or more compared to polyphenol oxidase activity of a wild-type grape plant cell. In some embodiments, the polyphenol oxidase activity is reduced by at least 95% or more compared to polyphenol oxidase activity of a wild-type grape plant cell.

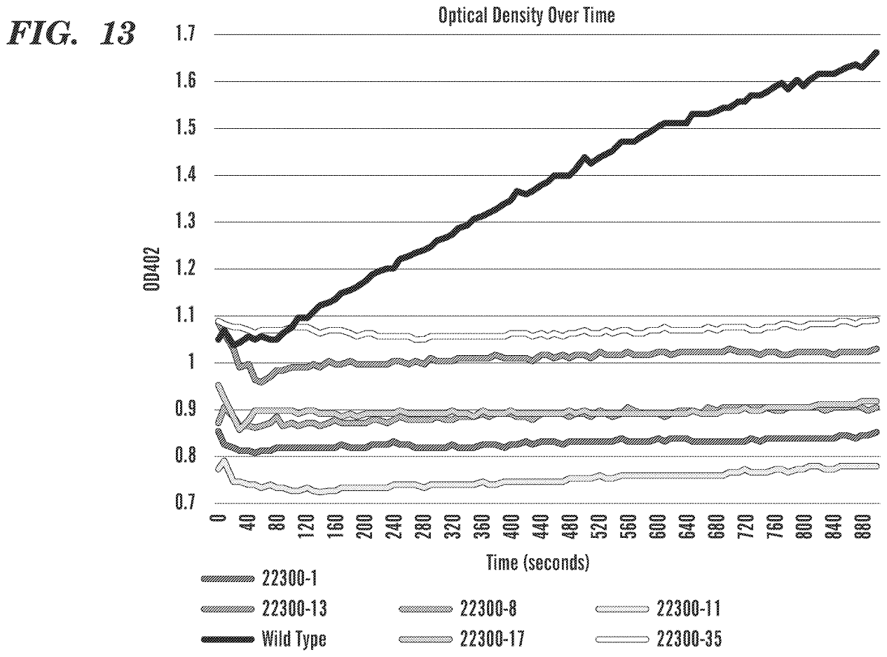

The reduction in total PPO activity may vary depending on a number of factors including, but not limited to, the tissue type, the developmental stage of a plant or plant material, the method of cultivation, the harvesting conditions, crushing or damage, the experimental conditions, and combinations and variations thereof. Exemplary total PPO activity is shown in FIGS. 13-15 for grape varieties with loss-of-function mutations described herein in comparison to a wild-type non-mutated variety. In some embodiments, the total PPO activity for total PPO protein in a grape cell, plant, plant part, seed, or fruit of the present disclosure is 0-5%, 5-10%, 5-15%, 10-20%, 20-30%, 30-40%, 40-50%, 50-60%, 60-70%, 70-80%, 80-90%, or 90-99%, or any range therein, of the wild-type total PPO activity. In some embodiments, the total PPO activity for any one or more PPO proteins or polypeptides in a grape cell, plant, plant part, seed, or fruit of the present disclosure is 0-5%, 5-10%, 5-15%, 10-20%, 20-30%, 30-40%, 40-50%, 50-60%, 60-70%, 70-80%, 80-90%, or 90-100%, or any range therein, of the wild-type total PPO activity. In some embodiments the total PPO activity is 5-15% of the wild-type total PPO activity.

In some embodiments, the grape cell, plant, plant part, seed, or fruit of the present disclosure has a PPO1 activity that is reduced by 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, less than 1%, or or any number or range therein, of the activity of PPO1 in wild-type grape cells, plants, plant parts, or fruits.

In some embodiments, the grape cell, plant, plant part, seed, or fruit of the present disclosure has a PPO2 activity that is reduced by 100%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, less than 1%, or any number or range therein, of the activity of PPO2 in wild-type grape cells, plants, plant parts, or fruits.

In some embodiments, the grape cell, plant, plant part, seed, or fruit of the present disclosure has a PPO3 activity that is reduced by 100%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, less than 1%, or any number or range therein, of the activity of PPO3 in wild-type grape cells, plants, plant parts, or fruits.

In some embodiments, the grape cell, plant, plant part, seed, or fruit of the present disclosure has a PPO4 activity that is reduced by 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, less than 1%, or any number or range therein, of the activity of PPO4 in wild-type grape cells, plants, plant parts, or fruits.

In some embodiments, the grape cell, plant, plant part, seed, or fruit of the present disclosure has a PPO5 activity that is reduced by 100%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, less than 1%, or any number or range therein, of the activity of PPO5 in wild-type grape cells, plants, plant parts, or fruits.

Methods of Generating Grape Cells and Plants with PPO Mutations

Another aspect of the present disclosure relates to a method of making a grape plant with reduced polyphenol oxidase (PPO) gene expression or activity. This method involves introducing one or more loss-of-function mutations into a nucleic acid sequence encoding a polyphenol oxidase (PPO) gene selected from one or more of PPO1, PPO2, PPO3, PPO4, and PPO5 of a grape plant cell, wherein the grape plant cell has reduced polyphenol oxidase gene expression or activity compared to a wild-type grape plant cell. The method further involves regenerating a plant from the grape plant cell.

This aspect of the present disclosure can be carried out with any of the embodiments disclosed herein.

Transient or stable insertion of recombinant DNA into a plant cell or genome may be used to generate genome modifications using CRISPR or other forms of genome editing. In some embodiments, such genome modifications are achieved without inserting exogenous DNA into the plant cell. In some embodiments, a ribonucleotide particle or ribonucleoprotein (RNP) complex is preassembled and delivered to a target grape plant cell without introducing exogenous DNA. Use of ribonucleoprotein complexes (RNP) for genome editing can eliminate integration of nucleic acid into the plant genome and obviate the need for backcrossing and screening of progeny.

In some embodiments, the RNP complex is prepared in vitro using a molar ratio of genome editing nuclease to gRNA of 1:7. In some embodiments, the molar ratio of genome editing nuclease to gRNA ranges from 3:1, 2:1, 1:1, 1:2, 1:3, 1:6, 1:7, 1:8, or 1:9, as non-limiting examples.

In some embodiments, a plurality of RNP complexes is used to enable genome editing of multiple genes for traits of interest. In some embodiments, each RNP complex of the plurality of RNP complexes comprises a genome-editing nuclease and a gRNA sequence, where the plurality of RNP complexes comprise different gRNA sequences targeting at least two different genes. In some embodiments, the plurality of RNP complexes comprise different gRNA sequences targeting at least 3 or more different genes. The methods of the present disclosure allow the simultaneous editing of multiple different gene targets without the need to combine them by breeding.

In some embodiments, introduction of RNP complexes into plants may be performed by introducing the RNP complexes into protoplasts. Protoplasts may be made by any means known in the art such as, but not limited to, methods described in Nakajima et al., “Embryonic Callus Induction and Agrobacterium-Mediated Genetic Tranformation of ‘Shine Muscat’ Grape,” Plant Biotechnol 37:185-194 (2020); Vandelle et al., “The Grapevine E3 Ubiquitin Ligase VriATL156 Confers Resistance against the Downy Mildew Pathogen Plasmopara viticola,” Int J. Mol Sci 22:940 (2021); Dutt et al., “Transgenic Plants from Shoot Apical Meristems of Vitis vinifera L. ‘Thompson Seedless’ via Agrobacterium-Mediated Transformation,” Plant Cell Rep 26:2101-2110 (2007); Dhekney et al., “Agrobacterium-Mediated Transformation of Embryogenic Cultures and Plant Regeneration in Vitis rotundifolia Michx. (Muscadine Grape),” Plant Cell Rep 27:865-872 (2007); Badim et al., “Constitutive expression of VviNAC17 Transcription Factor Significantly Induces the Synthesis of Flavonoids and Other Phenolics in Transgenic Grape Berry Cells,” Front Plant Sci 13:964621 (2022); Tricoli, “Grape Protoplast Isolation and Regeneration of Plants for Use in Gene Editing Technology,”” Renewal Progress Report for CDFA Agreement 18-0397 (2019) (static.cdfa.ca.gov/PiercesDisease/reports%2F2019%2FTricoli%20Renewal%20Report%202%2022%202019.pdf); Bertini et al., “Regeneration of Plants from Embryogenic Callus-Derived Protoplasts of Garganega and Sangiovese grapevine (Vitis vinifera L.) Cultivars,” Plant Cell Tiss Organ Cult 138,:239-246 (2019); Xu et al., “Isolation and Culture of Grape Protoplasts From Embryogenic Suspension Cultures and Leaves of Vitis Vinifera And Vitis Rotundifolia,” Acta Hortic 738:787-790 (2007), and U.S. Patent Publication No. 2023/0265451 each of which is hereby incorporated by reference in its entirety. In some embodiments, equal ratio of each RNP complex is incubated with the protoplasts. In some embodiments, 1 nmol gRNA is used per 10,000; 50,000; 100,000; 150,000; 200,000; 300,000 protoplasts; or any amount or range in between. In some embodiments, 1 nmol gRNA is used per 100,000 protoplasts.

In some embodiments, protoplast cells are transfected with genome editing components (e.g., RNP complexes). Plant protoplasts are enclosed only by a plasma membrane and will therefore take up macromolecules like RNP complexes. These protoplasts can be capable of regenerating whole plants. Transfection or transformation of protoplasts may be performed using any method known in the art including, but not limited to, polyethylene glycol treatment (Lelivelt et al., “Plastid Transformation in Lettuce (Lactuca sativa L.) by Polyethylene Glycol Treatment of Protoplasts,” Meth. Mol. Biol. 1132:317-330 (2014); Lelivelt et al., “Stable Plastid Transformation in Lettuce (Lactuca sativa L.),” Plant Mol. Biol. 58:763-774 (2005), each of which is hereby incorporated by reference in its entirety); using Sheen's protocol (Sheen, J. (2002) at URL genetics.mgh.harvard.edu/sheenweb/; Yoo & Sheen, “Arabidopsis Mesophyll Protoplasts: A Versatile Cell System for Transient Gene Expression Analysis,” Nat. Protocol. 2(7):1565-1572 (2007), and U.S. Patent Publication No. 2023/0265451, each of which is hereby incorporated by reference in its entirety), microinjection, gene gun delivery (RNP biolistics or proteolistics), electroporation, gold nanoparticles, starch nanoparticles, silica nanoparticles, and the like.

In some embodiments, said introducing comprises transfecting the plant cell with at least one ribonucleoprotein (RNP) complex comprising a guide RNA and a genome editing nuclease, and editing the plant cell's genome to induce the one or more loss-of-function mutations. In some embodiments, said introducing comprises transfecting the plant cell with two or more different RNP complexes. In some embodiments, the guide RNA comprises one or more of SEQ ID NOs:17-24.

In some embodiments, the protoplast cell genome is edited to induce loss-of-function mutations in one or more of PPO1, PPO2, PPO3, PPO4, and PPO5 genes as described herein. In some embodiments, the loss-of-function mutation is in the PPO2 gene. In some embodiments, the protoplast cells are cultured to make a grape cell with a loss-of-function mutation in the PPO2 gene, and one or more of the PPO1, PPO3, PPO4, and PPO5 genes.

In some embodiments, said introducing is carried out with respect to the PPO1, PPO2, PPO3, PPO4, and PPO5 gene. In some embodiments, said introducing is carried out with respect to the PPO2 gene and one or more of the PPO1, PPO3, PPO4, and PPO5 genes. In some embodiments, said introducing is carried out with respect to the PPO1, PPO2, PPO3 and PPO5 genes.

Another aspect of the present disclosure relates to a method of editing one or more PPO genes of a grape cell. This method involves introducing into a grape cell a polynucleotide construct comprising a first nucleic acid sequence encoding a gene editing nuclease; a first promoter that is functional in plants, wherein the first promoter is operably linked to the first nucleic acid sequence; a second nucleic acid sequence encoding one or more gRNA(s) targeting one or more of polyphenol oxidase PPO1, PPO2, PPO3, PPO4, and PPO5 genes to edit; and a second promoter that is functional in plants, operably linked to the second nucleic acid sequence.

This aspect of the present disclosure can be carried out with any of the embodiments disclosed herein.

Useful promoters for driving expression of the nucleic acid sequence encoding gRNAs targeting PPO genes of interest are any pol III and/or pol II promoters operable in plants including, but not limited to, an Arabidopsis thaliana U6 promoter, a 35S promoter, an Arabidopsis thaliana AtUBQ10 promoter, and a CsVMV promoter. The promoter that drives expression of the gRNA(s) may be the same or different than the promoter that drives expression of the gene editing nuclease. In some embodiments, the gene editing nuclease is a Cas9 nuclease, a MAD7 nuclease, a Cpf1 nuclease, or a chimeric nuclease. In some embodiments, the first promoter and the second promoter are each independently selected from AtU6 promoter, 35S promoter, and CsVMV promoter. In some embodiments, the method further comprises a third nucleic acid sequence encoding a selectable marker operably linked to a third promoter that is functional in plants.

Once genome editing has been performed, plants can be regenerated from the edited grape plant cell and grown into plants. Methods of cultivating protoplasts into plants may be done by any means known in the art. See, for example, methods described in Scintilla et al., “Regeneration of Plants from DNA-free Edited Grapevine Protoplasts,” bioRxiv doi.org/10.1101/2021.07.16.452503 (2021); Nakajima et al., “Embryonic Callus Induction and Agrobacterium-Mediated Genetic Tranformation of ‘Shine Muscat’ Grape,” Plant Biotechnol. 37:185-194 (2020); Vandelle et al., “The Grapevine E3 Ubiquitin Ligase VriATL156 Confers Resistance against the Downy Mildew Pathogen Plasmopara viticola,” Int. J. Mol. Sci. 22:940 (2021); Dutt et al., “Transgenic Plants from Shoot Apical Meristems of Vitis vinifera L. ‘Thompson Seedless’ via Agrobacterium-Mediated Transformation,” Plant Cell Rep 26:2101-2110 (2007); Dhekney et al., “Agrobacterium-Mediated Transformation of Embryogenic Cultures and Plant Regeneration in Vitis rotundifolia Michx. (Muscadine Grape),” Plant Cell Rep. 27:865-872 (2007); Badim et al., “Constitutive expression of VviNAC17 Transcription Factor Significantly Induces The Synthesis of Flavonoids and Other Phenolics in Transgenic Grape Berry Cells,” Front. Plant Sci. 13:964621 (2022); Tricoli, “Grape Protoplast Isolation and Regeneration of Plants for Use in Gene Editing Technology,” Renewal Progress Report for CDFA Agreement 18-0397 (2019) (static.cdfa.ca.gov/PiercesDisease/reports%2F2019%2FTricoli%20Renewal%20Report%202%2022%202019.pdf); Bertini et al., “Regeneration of Plants from Embryogenic Callus-Derived Protoplasts of Garganega and Sangiovese grapevine (Vitis vinifera L.) Cultivars,” Plant Cell Tiss. Organ Cult. 138:239-246 (2019); Xu et al., “Isolation and Culture of Grape Protoplasts From Embryogenic Suspension Cultures and Leaves of Vitis Vinifera And Vitis Rotundifolia,” Acta Hortic. 738:787-790 (2007), each of which is hereby incorporated by reference in its entirety.

In some embodiments, the methods of the present disclosure involve regenerating a plant from the plant cell having the genome edits in at least one of the PPO1, PPO2, PPO3, PPO4, and PPO5 genes. In some embodiments, a plant is regenerated from a plant cell having genome edits in at least 2, 3, 4, 5, or 6 different PPO genes.

The methods of the present disclosure include formation of somatic embryos (“SE”) comprising genome edited PPO genes. In some embodiments, proliferated calli are first subjected to a liquid pre-culture phase followed with a solid phase culture. In some embodiments, the liquid pre-culture calli are subjected to 2 weeks incubation in the dark in pre-culture media. In some embodiments, the proliferated calli are placed under dark in pre-culture media for 1 week, 2 weeks, 3 weeks, 4 weeks, 5 weeks, 6 weeks, 7 weeks, or more than 7 weeks. In some embodiments, the calli are subcultured to fresh media. In some embodiments, the calli are then transferred to fresh SE maturation media in the dark at 25° C. for 4 weeks. In some embodiments, the proliferated calli are placed under dark in SE maturation media for about 1 week, 2 weeks, 3 weeks, 4 weeks, 5 weeks, 6 weeks, 7 weeks, or more than 7 weeks in the dark. In some embodiments, the calli are transferred to fresh SE maturation media for an additional 3 weeks in the dark. In some embodiments, the proliferated calli are transferred to fresh SE maturation media and kept for about 1 week, 2 weeks, 3 weeks, 4 weeks, 5 weeks, 6 weeks, 7 weeks, or more than 7 weeks in the dark. In some embodiments, the proliferated calli are then cultured under the light for about 1 week, 2 weeks, 3 weeks, 4 weeks, 5 weeks, 6 weeks, 7 weeks, 8 weeks, 9 weeks, 10 weeks, 11 weeks, 12 weeks, or more than 12 weeks. In some embodiments, the proliferated calli are then cultured under the light for about 4 weeks to form SE. In some embodiments, the calli are sub-cultured to fresh media for about 1 week, 2 weeks, 3 weeks, 4 weeks, 5 weeks, 6 weeks, 7 weeks, 8 weeks, 9 weeks, 10 weeks, 11 weeks, 12 weeks, or more than 12 weeks to form SE.

A grape cell, plant part, plant, or fruit comprising genome edits as described herein may be identified by comparing the sequence of the region of the gene targeted by the RNP complex with the sequence from a control plant. A control plant or plant cell may comprise a wild-type plant or cell, i.e., of the same genotype as the starting material for the genome editing. In some embodiments, DNA is extracted from a grape cell, plant part, plant, or fruit, and the sequence around the target genome sites for the gRNA is evaluated. In some embodiments, Inference of CRISPR Edits (ICE) is used for analysis of genome edits (see Hsiau et al., “Inference of CRISPR Edits from Sanger Trace Data,” bioRxiv 251082 (2019), which is hereby incorporated by reference in its entirety). In some embodiments, the plant cell having genome edits is regenerated without the use of a selectable marker.

In some embodiments, the methods of the present disclosure include elongating the shoot. In some embodiments, elongating the shoot includes incubating a plant part, e.g., isolated regenerable cell cluster, that has grown a shoot of at least 0.5 cm, e.g., at least 0.6 cm, at least 0.7 cm, at least 0.8 cm, at least 0.9 cm, at least 1 cm, at least 1.2 cm, at least 1.4 cm, at least 1.6 cm, at least 1.8, including at least 2 cm on elongation medium. In some embodiments, the plant part is incubated under light for 1-6 weeks, e.g., 1-4 weeks, 2-4 weeks, 3-4 weeks, 4-5 weeks, 5-6 weeks, or longer than 6 weeks on the shoot elongation medium.

In some embodiments, the methods of the present disclosure include incubating a shoot on a suitable rooting medium. In some embodiments, vitrified shoot is incubated in the absence of any medium, e.g., in an empty petri dish, until vitrification is removed, before rooting.

In some embodiments, the method of mutating the PPO genes leaves no pest sequences in the genome of the grape plant or plant cell (such as Agrobacterium sequences or selectable marker sequences). In some embodiments, the grape plant, plant part, seed, or fruit is free of exogenous DNA. In some embodiments, the grape plant, plant part, seed, or fruit is free of plant pest sequences.

In some embodiments, the protoplast cell is transfected with gene editing components to edit the protoplast cell genome to alter the expression or activity of a gene. In some embodiments, the protoplast cell is cultured to make a grape cell with altered expression or activity of the gene. In some embodiments, the method further comprises regenerating the grape cell into a plant.

Phenotypes of Grapes with Loss-of-Function PPO Mutations and Grape Products Made Therefrom

Food products and beverages can be made from the plant, plant parts, seeds, and fruits according to the present disclosure. Non-limiting examples of food products include grapes, raisins, grape leaves, grape seeds, and grape oil. Non-limiting examples of beverages include grape juices and wines.

In some embodiments, grapes from grape plants of the present disclosure or food products and/or beverages made therefrom exhibit longer shelf life compared to food products and/or beverages made from grapes from wild-type grape plants under the same conditions.

Shelf life can be assessed by a number of factors including organoleptic scoring. For example and without limitation, organoleptic scores may be produced on a qualitative basis across several categories, including, for example and without limitation, color, off odor, aroma, moisture, texture, decay/mold, fruit discoloration, or taste. A total score combining values from each category provides an overall assessment of a plant, plant part, or fruit. In some embodiments, shelf life is scored in fresh grape fruit by cutting the grape fruit into pieces.

In some embodiments, shelf life is scored after processing the grape, food product, or beverage. In some embodiments the fruit is crushed and/or mashed, optionally mixed with other ingredients, and evaluated for organoleptic properties, especially color and aroma. In some embodiments, the shelf life is scored in a grape, food product, or beverage after storage under optimal conditions of light and temperature. In some embodiments, the shelf life is scored after storage under suboptimal conditions of light and temperature.

In some embodiments the harvested grape fruit has reduced friction damage after transport. As used herein, “friction damage” is characterized by an oxidation of the tissue that later turns brown and becomes necrotic. In some embodiments, the friction damage is reduced by 5%, 10%, 15%, 20%, 25%, 30%, 35%, 40%, 45%, 50%, 55%, 60%, 65%, 80%, 85%, 90%, 95%, or 100%, or any number or range therein, compared to a wild-type grape fruit after transport.

In some embodiments, the shelf life of the grape from grape plants of the present disclosure exhibits reduced failure rate at days after harvest compared to a wild-type variety under the same conditions as assessed by organoleptic scoring. As used herein, the “failure rate” means the percentage of replicates at a particular time point of a shelf life study that, assessed by organoleptic scoring, is unsuitable for marketability.

In some embodiments, the shelf life of the grape from grape plants of the present disclosure exhibits a reduced failure rate of 13 days after harvest as assessed by organoleptic scoring. In some embodiments, the grape exhibits 0% failure rate at 14 days post-harvest as assessed by organoleptic scoring. In some embodiments, the grape exhibits less than 5%, less than 10%, less than 20%, less than 30%, less than 40%, than 50% failure rate 14, 21 days, 28 days, or 35 days post-harvest as assessed by organoleptic scoring. In some embodiments, the grape exhibits less than 10% failure rate 12 days post-harvest as assessed by organoleptic scoring.

A recognized problem that is associated with harvested fruits is that the levels of plant phytochemicals, such as plant secondary metabolites, start to decrease almost immediately post-harvest. Such phytochemicals include vitamins, e.g., vitamins A, C, E, K, and/or folate, carotenoids such as beta-carotene, lycopene, the xanthophyll carotenoids such as lutein and zeaxanthin, phenolics comprising the flavonoids such as the flavonols (e.g., quercetin, rutin, caffeic acids), resveratrol, sugars, and other food products such as anthocyanins, among many others. In some embodiments, grapes harvested from grape plants described herein exhibit 5%, 10%, 15%, 20%, 25%⁰, 30%, 35%, 40%, 45%, 50%, 55%, 60%, 65%, 80%, 85%⁰, 90%, 95%, 100%, or any number or range therein, higher or more than 100% higher levels of plant phytochemicals compared to a wild-type variety.

In some embodiments, grape fruit, food products, and/or beverages of the present disclosure also exhibit higher levels of polyphenolics in comparison to a wild-type variety. Exemplary grape polyphenols include anthocyanins, flavanols, and resveratrol, which can have antioxidant, cardioprotective, anticancer, anti-inflammation, antiaging and antimicrobial properties.

In some embodiments, the level of polyphenolics is 5%, 10%, 15%, 20%, or more than 20% higher than a wild-type control. The grape fruit of the present disclosure may also retain higher levels of polyphenolics after harvest in comparison to a wild-type control. In some embodiments, the level of polyphenolics is 5%, 10%, 15%, 20%, 30%, 35%, 40%, 45%, 50%, 55%, 60%, 65%, 80%, 85%, 90%, 95%, 100%, or any number or range therein, or more than 100% higher levels of polyphenolics compared to a wild-type variety at 0, 7, 14, or 21 days after harvest. In some embodiments, the level of polyphenolics is 50% higher than wild-type levels.

A person of ordinary skill in the art would readily understand that the methods described herein may be modified and optimized for particular embodiments of choice. The following examples are intended to illustrate but not limit the scope of the invention.

### EXAMPLES

### Example 1—PPO Gene Identification in Grapes

Polyphenol oxidase (PPO) coding gene sequences were discovered in the annotated genome of Vitis vinifera Pinot Noir cultivar PN40024 (Jaillon et al., “The Grapevine Genome Sequence Suggests Ancestral Hexaploidization in Major Angiosperm Phyla,” Nature 449(7161):463-7 (2007), which is hereby incorporated by reference in its entirety). The genome sequence is available from NCBI (Genbank RefSeq GCF_000003745.3, which is hereby incorporated by reference in its entirety). The annotated Vitis vimifera PPO protein sequence (Genbank Accession No. NP_001268045.1, which is hereby incorporated by reference in its entirety) was used to identify other putative PPO and PPO-like genes using tBLASTn searches (E-value of 1e⁻¹⁰). These sequences were manually curated, as follows: the exon-intron gene structure was determined by comparing the genomic sequence with their corresponding predicted gene-model and translated amino acid sequence. Predicted PPO gene models were discarded if they lacked transcriptional evidence.

Sequence evaluation of the grape PPO genes indicated that PPO1, PPO2, PPO3, and PPO genes encoded PPO proteins with intact tyrosinase domains (FIG. 16), whereas the PPO4 gene encoded a PPO protein lacking a portion of the tyrosinase domain (FIG. 16). Due to the truncated tyrosinase domain, the PPO4 protein was predicted to lack or have low levels of PPO enzymatic activity. No intact open reading frame was identified based on the sequence of the PPO6 gene. This result indicated that PPO6 was a pseudogene not capable of encoding a functional PPO enzyme.

### Example 2—PPO Gene Expression in Grapes

RNA was extracted from non-altered and crushed fruit from grapes of four varieties, which included a red seeded variety, a black seedless variety, a green seedless variety, and a green Cotton Candy variety. RNA from three biological replicates per variety was extracted then cDNA was synthesized using standard protocols. Gene expression (GE) was analyzed using digital droplet PCR (MoGene, St. Louis, MO) for the grape PPO1, PPO2, PPO3, PPO4, PPO3, and PPO6 sequences (SEQ ID NOs: 2, 5, 8, 11, 14, and 16, respectively) using the primers and probes indicated in Table 1 (SEQ ID NOs:25-27 for PPO1; SEQ ID NOs:28-30 for PPO2; SEQ ID NOs:31-33 for PPO3; SEQ ID NOs:34-36 for PPO4; SEQ ID NOs:37-39 for PPO5; SEQ ID NOs:40-42 for PPO6). GE was normalized relative to GAPDH gene expression using the primers indicated in Table 1 (SEQ ID NOs:43-45) developed to the GAPDH coding region (SEQ ID NO:46). The amino acid sequence for grape GAPDH is shown in SEQ ID NO:47. Other genes such as ACT2 can be used to normalize gene expression using appropriate primer sequences. The coding sequence and amino acid sequences for ACT2 are shown in SEQ ID NO:48 and SEQ ID NO:49, respectively.

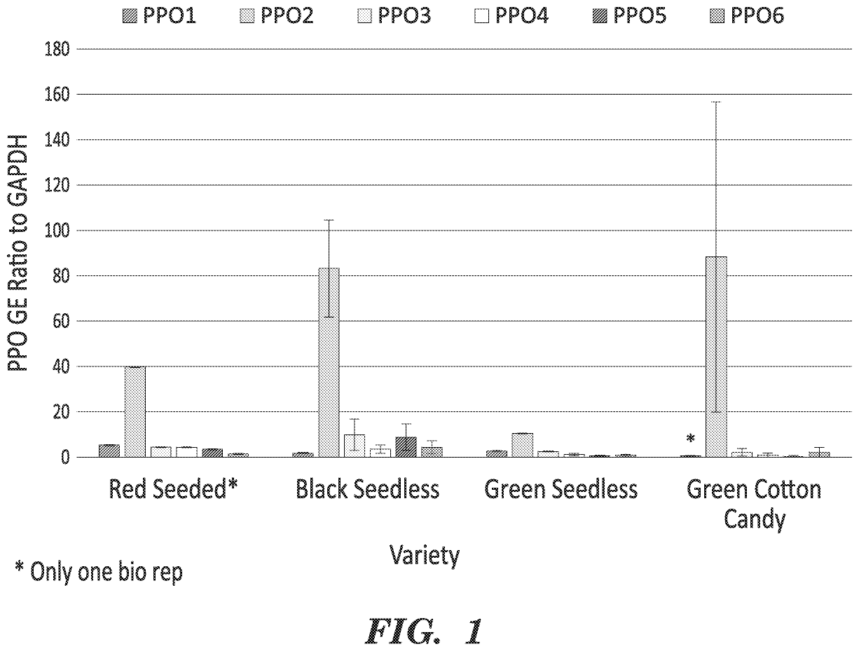

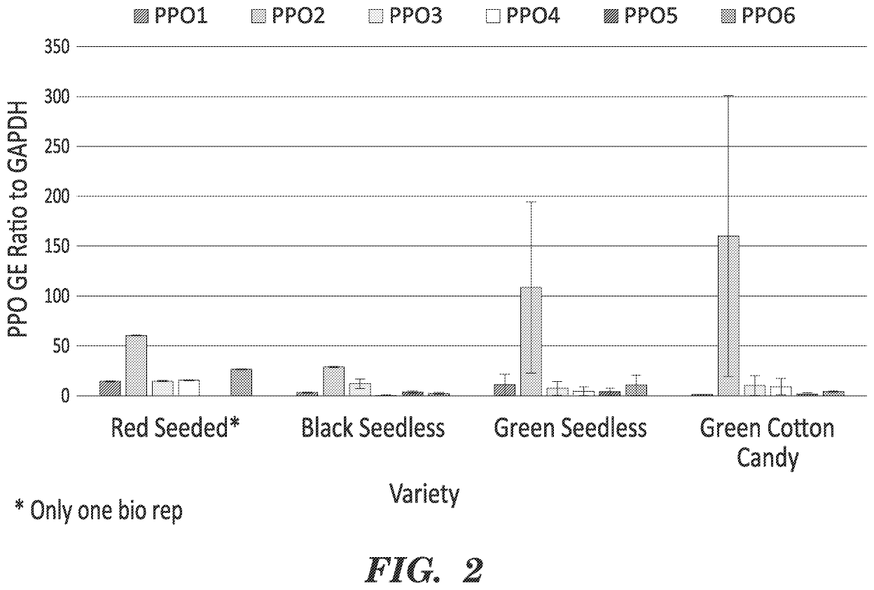

The graphs in FIG. 1 and FIG. 2 show the expression levels of the PPO1, PPO2, PPO3, PPO4, PPO5, and PPO6 genes in fruit of the four different grape varieties after normalization to GAPDH gene expression. PPO2 was the most highly expressed PRO gene in fruit from both the control (FIG. 1) and crushed grape samples (FIG. 2).

TABLE 1
PCR Primers & Probes for Gene Expression Analysis

| Target | | | SEQ ID | Amplicon |
|---|---|---|---|---|
| Gene | Primer Name | Primer Sequence | NO: | size (bp) |
| PPO1 | VvPPO1_F | GATGATGACCCGCGTAGTTT | 25 | 110 |
| | VvPPO1_Probe | CCCTTGGCAATAGGTGCAATGGAC | 26 | |
| | VvPPO1_R | GAAGCATGAACCTGGAGTTCTA | 27 | |
| PPO2 | VvPPO2_F | CAAACATCAGCCGCCAAAG | 28 | 103 |
| | VvPPO2_Probe | CGACTACACCTCACCGACAGCAAT | 29 | |
| | VvPPO2_R | GCCGGTACATAATGGAGAGATT | 30 | |
| PPO3 | VvPPO3_F | CACAATCCCTGATGACGAACTA | 31 | 106 |
| | VvPPO3_Probe | AAACAAATTGTGTCGGGTGCCACG | 32 | |
| | VvPPO3_R | GCGGTATGGGTAACCAAAGA | 33 | |
| PPO4 | VvPPO4_F | CCTAGTAAGGTGTCGTGCAATG | 34 | 110 |
| | VvPPO4_Probe | TGCGAATGGTGATCCAAACTCGGA | 35 | |
| | VvPPO4_R | CCAAGAAGCACATTCCTCCTATC | 36 | |
| PPO5 | VvPPO5_F | TCTTCCCTTTCCACCGTTTC | 37 | 102 |
| | VvPPO5_Probe | TAACGATCCCACCTTCGCTTTGCC | 38 | |
| | VvPPO5_R | GGGAATCCCAGCTCCAATAG | 39 | |
| PPO6 | VvPPO6_F | GGCGTTCTCATCGTAGAAGAC | 40 | 105 |
| | VvPPO6_Probe | CGTTTAGCGAATCTGGGTCCGTGA | 41 | |
| | VvPPO6_R | CGACCGTATGTGGATGCTATG | 42 | |
| GAPDH | VvGAPDH_F | GGACCCATCAGAAATCCCTTG | 43 | 113 |
| | VvGAPDH_Probe | AACAACCGAGAAGGCTTCTGCACA | 44 | |
| | VvGAPDH_R | TCTTGGCACCACCCTTTATG | 45 | |

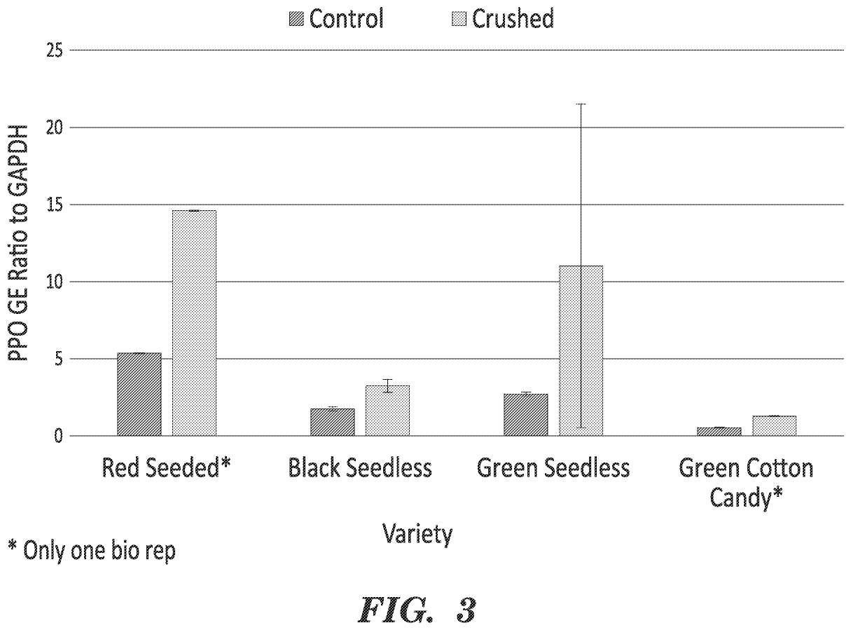

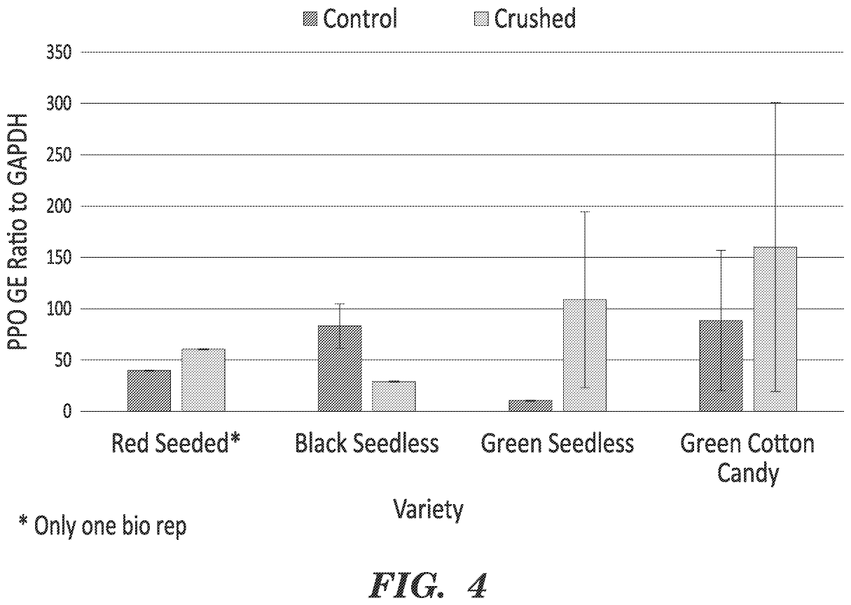

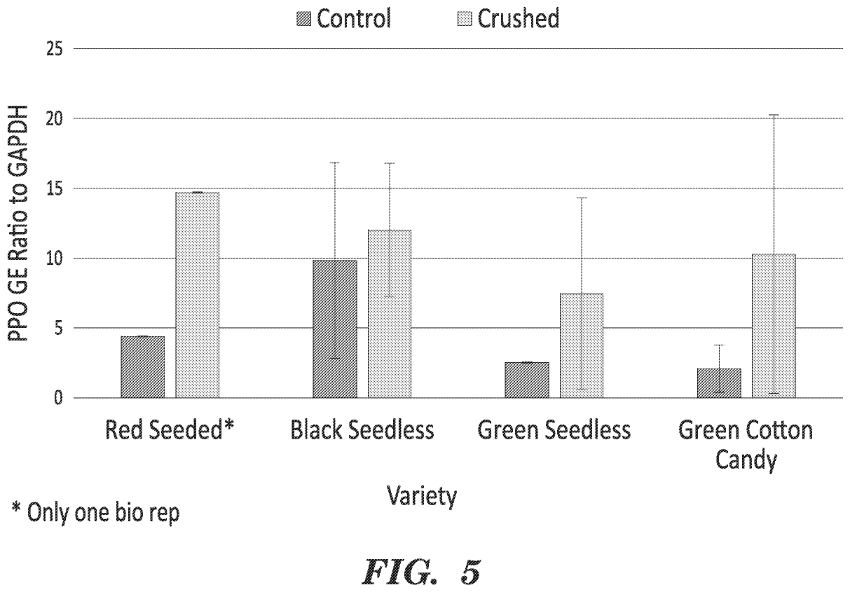

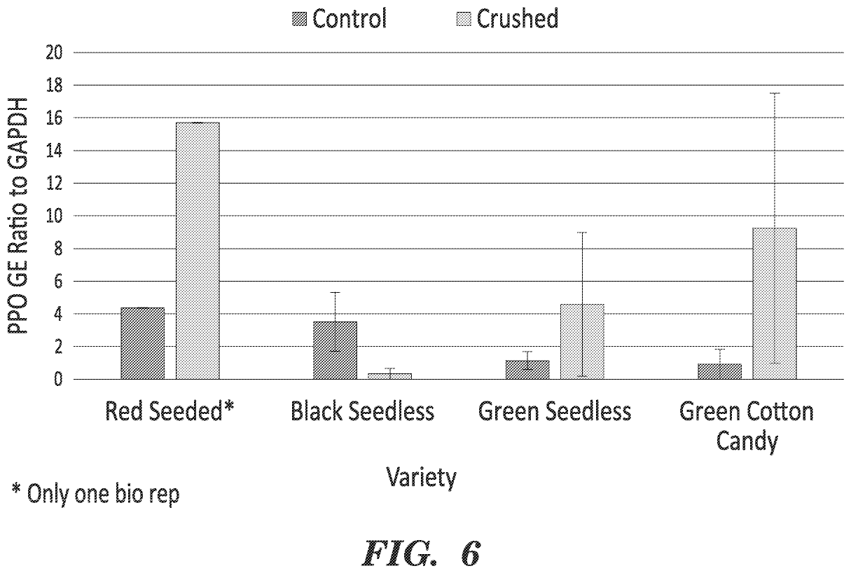

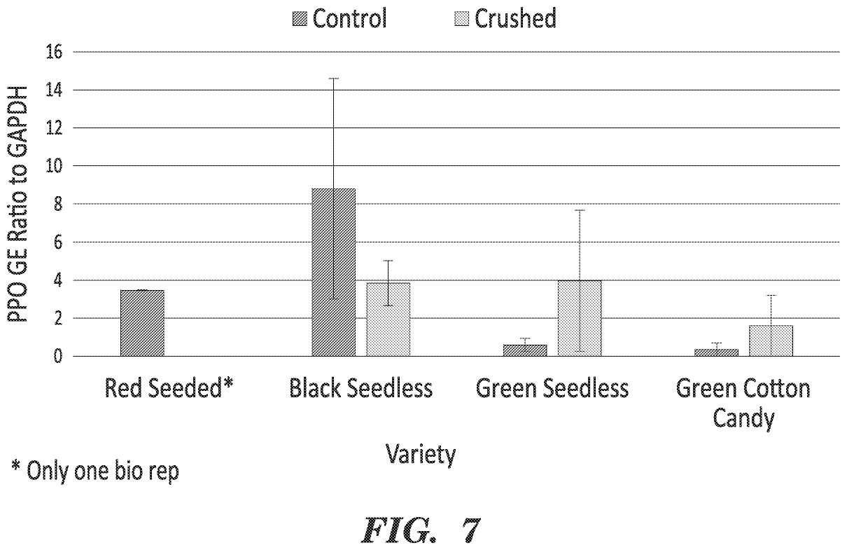

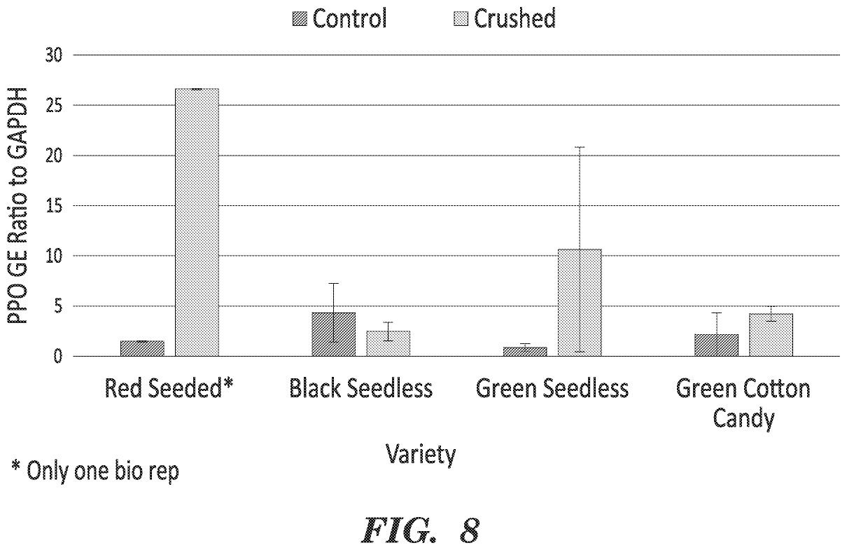

PPO1 and PPO3 gene expression generally increased in crushed fruit of grapes relative to the control (uncrushed) fruit of grapes (see FIG. 3 and FIG. 5, respectively). PPO2 expression increased after crushing fruit of grapes in three of the four varieties (FIG. 4) with PPO5 (FIG. 6) and PPO6 (FIG. 7) showing a similar trend.

### Example 3—Grape PPO Gene Editing

crRNA sequences targeting the tyrosinase domain of one or more PPO genes were identified using the “Find CRISPR sites” prediction tool from Geneious Prime software. crRNAs and tracrRNAs were then synthesized in vitro by a third-party provider (Integrated DNA Technologies (“IDT”), San Diego, CA).

Additional crRNAs and gRNAs can be developed and tested for editing efficiency. Software such as Geneious Prime can be used to select crRNAs that specifically target a particular PPO gene in order to edit only that gene. Alternatively, crRNAs can be identified using sequences that are identical in multiple PPO genes to target more than one PPO gene for genome editing at the same time. Additional gRNA sequences can be developed that are directed to one or more of the grape PPO genes in any part of the various PPO gene sequences using similar methods.

Two-part guide RNAs were assembled by mixing an equal amount of a crRNA comprising one of SEQ ID NOs: 17-24 with tracrRNA (IDT) and incubating the two RNA molecules together at 95° C. for 5 minutes. Once cooled down at room temperature to form the gRNAs, ribonucleoprotein particles (RNPs) were assembled by association of the gRNAs with SpCas9 genome editing nuclease using a molar ratio of genome editing nuclease to gRNA of 1:7. Cas9 protein and gRNAs were incubated together for 5 minutes at room temperature to allow RNP formation before being used for transfection. Eight different RNP mixtures were prepared, each with a gRNA comprising one of SEQ ID NOs: 17-24.

These various gRNA were evaluated for editing efficiency by transient transfection of ~100,000 protoplasts of grape variety Colombard with each RNP mixture. Transfection of protoplasts with RNPs was performed as described in U.S. Patent Publication No. 2023/0265451 (which is hereby incorporated by reference in its entirety) with modifications. Briefly, grape protoplasts were prepared from embryogenic callus and adjusted to a density of 1×10⁷ protoplast per ml in MG media. An aliquot of 100 μl (1×10⁶ protoplasts) was combined with 25 μl RNP mixture followed immediately by 125 μl of freshly prepared PEG 4000. Protoplasts were incubated for 12-20 minutes and transfections were stopped by adding 5.5-6× volume of W5 solution then mixed gently. Protoplasts were gently pelleted, washed 1-3 times, and resuspended in alginate solution (2.5-3.5%) and expelled dropwise or plated into calcium-containing media with mannitol. Protoplasts were incubated with liquid culture media The suspension was incubated at 50 rpm at 25° C. in the dark. The mannitol concentration in the medium was reduced by 50% at 7-14 days and 14-28 days post embedding.

For transient transfection, the protoplasts were cultured for 4 days. The protoplasts were then collected and DNA was isolated using standard extraction protocols. Genomic DNA concentrations were estimated using a nanodrop 8000 (Thermo Fisher Scientific, Waltham, MA) then diluted for further analysis.

After genomic DNA from the protoplast was isolated, PCR of PPO genes was performed, and PCR products were sequenced. Efficiencies for RNPs with gRNAs comprising one of SEQ ID NOs: 17-24 are shown in Table 2. In some cases, more than one PPO gene shared sequence identity matching the position targeted by the crRNA (i.e., matching 20/20 nucleotides), but in the absence of an NGG Protospacer Adjacent Motif (PAM) adjacent to the target sequence, no editing would occur. Editing was expected to be most efficient for PPO genes having 100% sequence similarity and an NGG PAM site adjacent to the target sequence. These PPO genes are identified as targeted PPO genes in Table 2 below.

TABLE 2
gRNA Editing Efficiencies in Transfected Protoplasts

| Grape PPO Gene(s) | Sequence of gRNA | PPO Gene Editing |
|---|---|---|
| Targeted | Targeting PPO Genes | Efficiency |
| PPO2 (matches 20/20 nt) | GCCUGUUGCAUGAAGCUCCG | 81% |
| PPO5 (matches 19/20 nt) | (SEQ ID NO: 17) | |
| PPO2 (matches 20/20 nt) | UCAUUGCGCCUACUGCAAUG | 28% |
| | (SEQ ID NO: 18) | |
| PPO2 (matches 20/20 nt) | GAUCAUUAAUCAACUUUCCC | 75% |
| | (SEQ ID NO: 19) | |
| PPO2 (matches 20/20 nt) | GGCAAAGGCGAAUGGAUCAG | 86% |
| | (SEQ ID NO: 20) | |
| PPO1 (matches 20/20 nt) | AUGAACCUGGAGUUCUAGGU | 0% - no editing detected |
| PPO3 (matches 20/20 nt) | (SEQ ID NO: 21) | |
| PPO4 (matches 20/20 nt) | | |
| PPO1 (matches 20/20 nt) | UGAAGUAGAGAUAGUAACGG | 76.5% |
| PPO3 (matches 20/20 nt) | (SEQ ID NO: 22) | |
| PPO1 (matches 20/20 nt) | GGGGAACUAGCAUAGAUGGU | 52% |
| PPO3 (matches 20/20 nt) | (SEQ ID NO: 23) | |
| PPO1 (matches 20/20 nt) | GUGAUCGAUCUCGACUACGA | 68% |
| PPO3 (matches 20/20 nt) | (SEQ ID NO: 24) | |

Based on the gene-editing efficiencies, RNPs with gRNAs comprising SEQ ID NO:17 and SEQ ID NO:22 were co-transfected into grape protoplasts as described above. Grape protoplasts from multiple grape varieties were transfected with RNPs in multiple different experiments. The grape varieties included Colombard (a white wine grape), Pixie (a dwarf variety derived from Pino Meunier, a red wine grape), and Malbec (a red wine grape).

Grape protoplasts were regenerated into plants from the Colombard variety based on established protocols described in Scintilla et al., “Regeneration of Plants from DNA-free Edited Grapevine Protoplasts,” bioRxiv doi.org/10.1101/2021.07.16.452503 (2021), which is hereby incorporated by reference in its entirety.

Protoplasts isolated from embryogenic cultures of Chardonnay 17 (Char17, a white wine grape) and Sauvignon Blanc (SB, a white wine grape) were transfected with RNP with a gRNA directed to PPO2 (SEQ ID NO:17 or SEQ ID NO:19) using the method described above.

After transfection, a subset of protoplasts were harvested and used for CRISPR next generation sequencing (“NGS”), while most of the protoplasts were embedded into alginate for culturing. The CRISPR NGS data revealed 15% gene editing of PPO2 in Char17 and 27% in SB varieties. These embedded protoplasts will be regenerated into grape plants using the methods described above and evaluated for PPO2 gene edits and PPO activity. Transfection and regeneration of plants from genome edited protoplasts of Pixie, Malbec, Chardonnay 7, Sauvingnon Blanc, Grenache, and Gruner Veltliner is ongoing.

### Example 4—PPO Gene Mutations

Genomic DNA was extracted from leaf tissue collected from fourteen regenerated genome edited Colombard grape plants.

The region of the grape PPO gene targeted by the gRNA was PCR amplified using Phusion Plus DNA Polymerase (Thermo Fisher Scientific, Waltham, MA) and a specific pair of primers for each target gene (see Table 3 below, SEQ ID NOs:50-51, or SEQ ID NOs:52-53 for PPO1; SEQ ID NOs:54-55, SEQ ID NOs:56-57, SEQ ID NOs:58-59, SEQ ID NOs:60-61, or SEQ ID NOs:62-63 for PPO2; SEQ ID NOs:64-65 for PPO3, and SEQ ID NOs:66-67 for PPO1) using 30 ng/2 μl genomic DNA following the manufacturer instructions. PCR reactions were run using 1100 Thermal Cycler (Bio-Rad, Hercules, CA). Amplicon sizes are based on the wild-type gene sequence.

TABLE 3
PCR Primers for Amplification of Genome Edited Targeted Genes

| Target | | Primer | | SEQ ID | Amplicon |
|---|---|---|---|---|---|
| Gene | Primer Name | ID | Primer Sequence | NO: | size (bp) |
| PPO1 | VvPPO1_Tyrosinasc_AT_F | BD16 | GCAGAAAGCTCTTCCTGATG | 50 | 254 |
| | VvPPO1_AT_DS_G1-2_R | BD95 | GCATATACATGCCATCAGGG | 51 | |
| PPO1 | VvPPO1_AT_DS_G3-4_F | BD96 | TCCACCGTTACTATCTCTACTTCA | 52 | 260 |
| | VvPPO1_AT_DS_G3-4_R | BE01 | CATGATTGCCAGATTGTCG | 53 | |
| PPO2 | VvPPO2_AT_DS_G4_F | BE02 | AACCTCTAGCTCCAAAAGTGG | 54 | 230 |
| | VvPPO2_AT_DS G4_R | BE03 | AATCTATGATCTTGCTGCTCACT | 55 | |
| PPO2 | VvPPO2_AT_DS_G1-2_F | BE04 | AGTGAGCAGCAAGATCATAGATT | 56 | 249 |
| | VvPPO2_AT_DS_G1-2_R | BE05 | CATGAATTGTGGACTTGGAG | 57 | |
| PPO2 | VvPPO2_AT_DS_G3_F | BE06 | TCCAAGTCCACAATTCATGG | 58 | 200 |
| | VvPPO2_AT_DS G3_R | BE07 | TTTGGCGTCGCGAAGCTT | 59 | |
| PPO2 | VvPPO2_AT_DS_G3_F | BE06 | TCCAAGTCCACAATTCATGG | 60 | 200 |
| | VvPPO2_AT_DS_G3_R | BE07 | TTTGGCGTCGCGAAGCTT | 61 | |
| PPO2 | VvPPO2_AT_DS_G3_F | BE06 | TCCAAGTCCACAATTCATGG | 62 | 200 |
| | VvPPO2_AT_DS_G3_R | BE07 | TTTGGCGTCGCGAAGCTT | 63 | |
| PPO3 | VvPPO4_5′_AT_F | BD24 | ATGGCTTCTTTGCCTTGGT | 64 | 1,163 |
| | VvPPO3_5′_AT_R | BD22 | CTATAGTTTTCCATATATTCCACAT | 65 | |
| | | | TC | | |
| PPO5 | VvPPO5_5′_AT_F | BD26 | CAAGTGTTTTCTCGACATCCCT | 66 | 1,000 |
| | VvPPO5_5′_AT_R | BD27 | TGTGGGTTCGGTGAAATC | 67 | |

Next generation sequencing (NGS) has been used to deeply sequence the specific PCR products for each target gene and assess gene editing frequency by comparing the number of pair reads showing a mutation (substitutions, insertions, deletions, or combinations) at the target site to the number of pair reads depicting a wild-type pattern. NGS was performed by the Center for Computational and Integrative Biology DNA Core Facility at Massachusetts General Hospital, Boston, USA using standard protocols.

Alignments of PPO gene sequences having genome edited mutations are shown in FIGS. 9A-B for PPO1, FIGS. 10A-B for PPO2, FIGS. 11A-B for PPO3, and FIGS. 12A-B for PPO3. The edited sequences were aligned to the coding sequences of PPO1 (SEQ ID NO:2), PPO2 (SEQ ID NO:5), PPO3 (SEQ ID NO:8), or PPO5 (SEQ ID NO:14).

Table 4 shows the mutations introduced by genome editing in PPO genes that are present in each Colombard grape line. Deletions are indicated by a “D” followed by the number of nucleotides deleted. Different mutations with the same number of nucleotides deleted, but at different nucleotide positions, are indicated with a dash followed by a number. For example, a mutation with a first deletion of 5 nucleotides is named “D5-1”. A second mutation with a different deletion of 5 nucleotides is named “D5-2”. Heterozygous lines having a different mutation in each chromosomal allele of a specific gene are indicated with a “/” symbol. For example, line 22300-8 grape with a genotype of “D117/D118” has a mutation with a deletion of 117 nucleotides in one allele of PPO2 (SEQ ID NO:73), and another mutation with a deletion of 118 nucleotides in the other allele of PPO2 (SEQ ID NO:72). All other mutation lines were homozygous for the indicated mutation or wild-type allele. Lines 22300-1, 8, 11, and 35 also have undetermined (Und) mutations in PPO1, PPO3 and/or PPO3 as indicated. The deletion and insertion mutations of SEQ ID NOs:68-79 are loss-of-function mutations, and more specifically knockout mutations, since they eliminate amino acids in the tyrosinase domain (FIG. 16) and/or cause a frameshift leading to a premature stop codon in the coding sequence.

Specific enzymatic activity of PPO was measured in a number of PPO genome edited lines and compared to wild-type as shown in Table 4 and discussed in Example 5.

TABLE 4
PPO Mutation Combinations in Various Grape Lines

| | | | | | PPO |
|---|---|---|---|---|---|
| Plant | | | | | Specific |
| Line | PPO1 | PPO2 | PPO3 | PPO5 | Activity |
| 22300 | wild-type | wild-type | wild-type | wild-type | 1003 |
| | (SEQ ID | (SEQ ID | (SEQ ID | (SEQ ID | |
| | NO: 2) | NO: 5) | NO: 8) | NO: 11) | |
| 22300-1 | D126-1 | D118 | D126-2 | NS | 79 |
| | (SEQ ID | (SEQ ID | (SEQ ID | | |
| | NO: 68) | NO: 71) | NO: 74) | | |
| 22300-8 | Und | D117/D118 | Und | I1-2 | 129 |
| | | (SEQ ID | | (SEQ ID | |
| | | NO: 72)/ | | NO: 77) | |
| | | (SEQ ID | | | |
| | | NO: 71) | | | |
| 22300-11 | Und | D117/D118 | Und | I1-2 | 65 |
| | | (SEQ ID | | (SEQ ID | |
| | | NO: 72)/ | | NO: 77) | |
| | | (SEQ ID | | | |
| | | NO: 71) | | | |
| 22300-12 | D5-1/D5-2 | I1-1 | D5-3/D5-4 | NS | |
| | (SEQ ID | (SEQ ID | (SEQ ID | | |
| | NO: 69)/ | NO: 73) | NO: 75)/ | | |
| | (SEQ ID | | (SEQ ID | | |
| | NO: 70) | | NO: 76) | | |
| 22300-13 | D5-1/D5-2 | D118 | D5-3/D5-4 | NS | 94 |
| | (SEQ ID | (SEQ ID | (SEQ ID | | |
| | NO: 69)/ | NO: 71) | NO: 75) | | |
| | (SEQ ID | | (SEQ ID | | |
| | NO: 70) | | NO: 76) | | |
| 22300-17 | D5-1/D5-2 | D118 | D5-3/D5-4 | NS | 83 |
| | (SEQ ID | (SEQ ID | (SEQ ID | | |
| | NO: 69)/ | NO: 71) | NO: 75)/ | | |
| | (SEQ ID | | (SEQ ID | | |
| | NO: 70) | | NO: 76) | | |
| 22300-20 | D5-1/D5-2 | I1-1 | D5-3/D5-4 | NS | |
| | (SEQ ID | (SEQ ID | (SEQ ID | | |
| | NO: 69)/ | NO: 73) | NO: 75)/ | | |
| | (SEQ ID | | (SEQ ID | | |
| | NO: 70) | | NO: 76) | | |
| 22300-35 | Und | D117/D118 | Und | Und | 56 |
| | | (SEQ ID | | | |
| | | NO: 72)/ | | | |
| | | (SEQ ID | | | |
| | | NO: 71) | | | |
| 22300-51 | D5-1/D5-2 | I1-1 | D5-3/D5-4 | NS | |
| | (SEQ ID | (SEQ ID | (SEQ ID | | |
| | NO: 69)/ | NO: 73) | NO: 75)/ | | |
| | (SEQ ID | | (SEQ ID | | |
| | NO: 70) | | NO: 76) | | |
| 22300-61 | D5-1/D5-2 | I1-1 | D5-3/D5-4 | D2/D4 | |
| | (SEQ ID | (SEQ ID | (SEQ ID | (SEQ ID | |
| | NO: 69)/ | NO: 73) | NO: 75)/ | NO: 78/ | |
| | (SEQ ID | | (SEQ ID | SEQ ID | |
| | NO: 70) | | NO: 76) | NO: 79) | |
| 22300-62 | D5-1/D5-2 | I1-1 | D5-3/D5-4 | NS | |
| | (SEQ ID | (SEQ ID | (SEQ ID | | |
| | NO: 69)/ | NO: 73) | NO: 75)/ | | |
| | (SEQ ID | | (SEQ ID | | |
| | NO: 70) | | NO: 76) | | |

Und-mutation present with undetermined sequence;
NS-not sequenced

### Example 5—PPO Enzymatic Activity Assay

For evaluation of enzymatic PPO activity, 43-68 mg of in-vitro grown leaf tissue was collected, sandwiched between wax paper, and rolled over three times with a glass hybridization tube in different directions, applying ~1.5 kg of force, to crush tissue. Samples were transferred to 2 ml lock tubes with two mm glass beads and left for 24 hrs at room temperature. The PPO activity assay was performed in a flat bottom 96-well assay plate containing either 10 mM pyrocatechol or 10 mM 4-methylcatechol substrate in 100 mM sodium phosphate pH 5 buffer and 0.2% SDS and thirty microliter of crude protein extract was quickly added to the substrate using a multichannel pipet and measurement of PPO activity at 405 nm was monitored. As shown in FIG. 13 using the 4-methylcatechol substrate, a kinetic window was chosen between 5-6 minutes, and the change in optical density (“OD”) over time was recorded. The OD of the wild-type (top line) conspicuously increased at a faster rate than the PPO-edited lines (22300-1, -8, -11, -13, -17, and -35) indicating that the PPO-edited lines had low PPO activity.

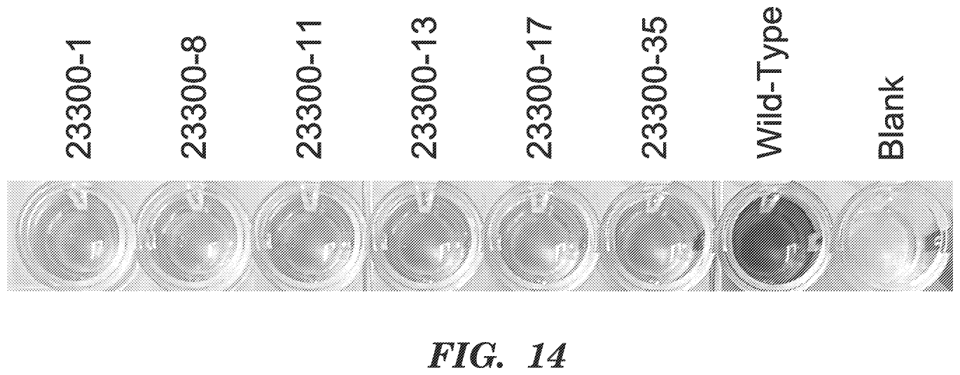

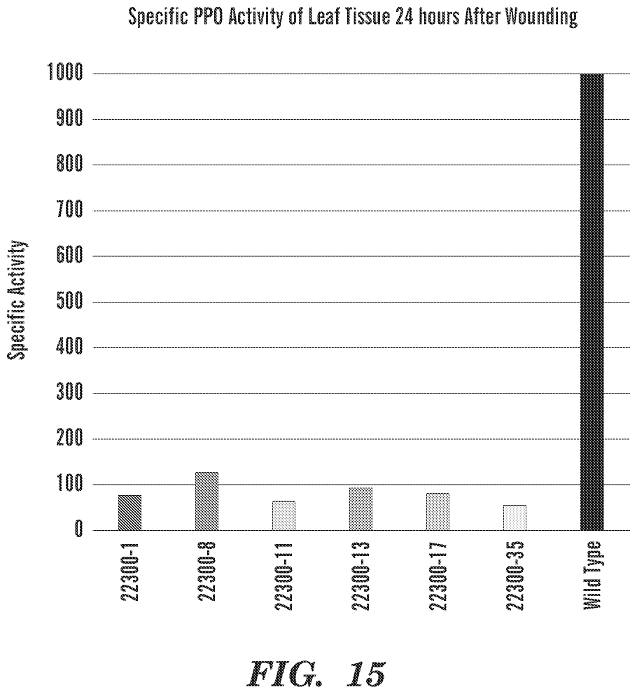

A photograph of the PPO assay using the pyrocatechol substrate and taken after 18 hours is shown in FIG. 14. As in the other experiments, an in vitro grown wild-type Colombard plant (“22300-WT”) was used as a control sample in comparison to the PPO edited lines (22300-1, -8, -11, -13, -17, and -35). The control sample produced a dark brown color, whereas the PPO-edited lines had a light yellow color indicating that the PPO-edited lines had low PPO activity.

The specific activity of PPO in a wild-type (Colombard-22300) in comparison to PPO edited lines (22300-1, -8, -11, -13, -17, and -35) is shown in FIG. 15. One Unit (U)=0.001 OD per minute. Specific activity is defined as one U per mg protein. A one-minute kinetic window was chosen between 5 to 6 minutes of the PPO assay and protein concentration was determined by Bradford assay. PPO activity of the wild-type was 7.8 to 17.9 times higher than the PPO gene edited lines. Specific activity values are also shown in Table 4.

### Example 6. Grape PPO & Other Sequences

Vitis vinifera PPO gene, coding, and amino acid sequences are shown in Table 5 below. The tyrosinase domain is indicated in bold text for SEQ ID NOs:2, 3, 5, 6, 8, 9, 11, 12, 14, and 15. SEQ ID NO: 13 was updated with additional sequence at the 5′ end of the gene, based on the sequence of SEQ ID NO:14.

TABLE 5
Grape PPO & Other Sequences

| SEQ ID | | |
|---|---|---|
| NO: | Gene | Sequence |
| 1 | Vitis | AGCTATGGCT TCTTTGCCTT GGTCGCTCAC AACCTTCACC GCCATCGCCA |
| | vinifera | ACACCACCAA CATTTCAGCC TTCCCACCTT CTCCCTTGTT TCAAAGGGCT |
| | PPO1 | TCTCATGTCC CCGTAGCCAG AAACCGAAGC CGCAGATTTG CTCCTAGTAA |
| | nucleotide | GGTGTCGTGC AATTCTGCGA ATGGTGATCC AAACTCGGAT TCTACCTCCG |
| | sequence | ACGTTCGAGA AACTTCCTCA GGGAAGTTAG ATAGGAGGAA TGTGCTTCTT |
| | | GGCATAGGAG GGCTGTATGG TGCTGCTGGC GGTCTCGGCG CCACTAAGCC |
| | | ATTAGCCTTT GGGGCTCCCA TCCAGGCACC GGATATATCC AAGTGTGGTA |
| | | CCGCCACCGT GCCTGATGGT GTAACGCCCA CAAATTGTTG CCCGCCAGTC |
| | | ACCACAAAGA TTATAGATTT CCAGCTACCT TCCTCAGGTT CCCCCATGCG |
| | | TACCAGGCCA GCTGCTCACT TGGTCAGCAA AGAGTACTTA GCCAAGTATA |
| | | AAAAGGCCAT TGAGCTGCAG AAAGCTCTTC CTGATGATGA CCCGCGTAGT |
| | | TTCAAGCAAC AGGCTAATGT CCATTGCACC TATTGCCAAG GGGCTTATGA |
| | | TCAGGTTGGG TATACCGACC TAGAACTCCA GGTTCATGCT TCATGGCTTT |
| | | TCCTCCCTTT CCACCGTTAC TATCTCTACT TCAATGAGAG AATTCTTGCA |
| | | AAGTTGATCG ACGATCCCAC CATCGCTTTG GCCTATTGGG CTTGGGATAA |
| | | CCCTGATGGC ATGTATATGC CGACCATCTA TGCTAGTTCC CCATCATCAC |
| | | TCTACGACGA GAAGCGCAAC GCCAAGCACC TGCCTCCGAC TGTGATCGAT |
| | | CTCGACTACG ATGGCACCGA ACCCACAATC CCTGATGACG AACTAAAAAC |
| | | CGACAATCTG GCAATCATGT ACAAACAAAT TGTGTCGGGT GCCACGACTC |
| | | CTAAGCTTTT CCTTGGTTAC CCATACCGCG CCGGCGATGC GATTGACCCT |
| | | GGAGCGGGTA CCCTTGAGCA CGTCCCACAT AATATAGTCC ACAAATGGAC |
| | | TGGTCTTGCT GATAAGCCTA GTGAGGACAT GGGAAACTTC TATACTGCCG |
| | | GCAGAGACCC CATATTCTTC GGTCACCACG CCAATGTCGA TCGGATGTGG |
| | | AATATATGGA AAACTATAGG AGGTAAAAAT AGAAAGGATT TCACGGATAC |
| | | GGATTGGCTT GACGCCACGT TCGTCTTCTA CGACGAGAAC AAACAACTTG |
| | | TTAAAGTCAA GGTCTCGGAC TGTGTCGACA CTTCCAAGCT GAGATACCAA |
| | | TATCAGGATA TTCCTATTCC ATGGCTACCA AAAAAGACGA AGGCCAAAGC |
| | | GAAGACGACC ACCAAAAGTT CCAAGTCGGG AGTAGCGAAA GCGGCCGAAC |
| | | TCCCAAAGAC GACGATCAGC AGCATCGGAG ACTTCCCAAA AGCTCTTAAC |
| | | TCAGTGATAA GAGTAGAAGT TCCAAGGCCA AAGAAATCAA GAAGCAAGAA |
| | | GGAGAAAGAG GATGAGGAAG AGGTGTTACT GATAAAAGGA ATAGAGCTAG |
| | | ATAGAGAGAA TTTCGTGAAG TTTGATGTGT ACATCAACGA CGAAGATTAT |
| | | TCAGTGAGTA GGCCTAAGAA TAGTGAGTTT GCAGGAAGCT TTGTGAACGT |
| | | ACCACACAAG CATATGAAAG AAATGAAGAC GAAGACCAAT CTGAGGTTCG |
| | | CGATAAATGA GCTGTTAGAG GACTTGGGAG CCGAAGATGA TGAGAGTGTG |
| | | ATCGTGACTA TAGTCCCTCG TGCTGGGGGC GATGATGTCA CCATTGGTGG |
| | | AATTGAGATC GAGTTTGTTT CCGATTGATC CCATCTTTCA ATGATTATCC |
| | | ATTATATGTA TGTATCAGGT AAGTCACATC TTTATGTGAC TAATGGAAAA |
| | | TGTGAGACTT CTCTGTACTC TCCCGTCAAG TCTTTTATTA ATTTAGAGCT |
| | | GTTTGGTTTA AAAAAAAAAA AAAAAAAA |
| 2 | Vitis | ATGGCTTCTT TGCCTTGGTC GCTCACAACC TTCACCGCCA TCGCCAACAC |
| | vinifera | CACCAACATT TCAGCCTTCC CACCTTCTCC CTTGTTTCAA AGGGCTTCTC |
| | PPO1 | ATGTCCCCGT AGCCAGAAAC CGAAGCCGCA GATTTGCTCC TAGTAAGGTG |
| | coding | TCGTGCAATT CTGCGAATGG TGATCCAAAC TCGGATTCTA CCTCCGACGT |
| | sequence | TCGAGAAACT TCCTCAGGGA AGTTAGATAG GAGGAATGTG CTTCTTGGCA |
| | | TAGGAGGGCT GTATGGTGCT GCTGGCGGTC TCGGCGCCAC TAAGCCATTA |
| | | GCCTTTGGGG CTCCCATCCA GGCACCGGAT ATATCCAAGT GTGGTACCGC |
| | | CACCGTGCCT GATGGTGTAA CGCCCACAAA TTGTTGCCCG CCAGTCACCA |
| | | CAAAGATTAT AGATTTCCAG CTACCTTCCT CAGGTTCCCC CATGCGTACC |
| | | AGGCCAGCTG CTCACTTGGT CAGCAAAGAG TACTTAGCCA AGTATAAAAA |
| | | GGCCATTGAG CTGCAGAAAG CTCTTCCTGA TGATGACCCG CGTAGTTTCA |
| | | AGCAACAGGC TAATGTCCAT TGCACCTATT GCCAAGGGGC TTATGATCAG |
| | | GTTGGGTATA CCGACCTAGA ACTCCAGGTT CATGCTTCAT GGCTTTTCCT |
| | | CCCTTTCCAC CGTTACTATC TCTACTTCAA TGAGAGAATT CTTGCAAAGT |
| | | TGATCGACGA TCCCACCATC GCTTTGGCCT ATTGGGCTTG GGATAACCCT |
| | | GATGGCATGT ATATGCCGAC CATCTATGCT AGTTCCCCAT CATCACTCTA |
| | | CGACGAGAAG CGCAACGCCA AGCACCTGCC TCCGACTGTG ATCGATCTCG |
| | | ACTACGATGG CACCGAACCC ACAATCCCTG ATGACGAACT AAAAACCGAC |
| | | AATCTGGCAA TCATGTACAA ACAAATTGTG TCGGGTGCCA CGACTCCTAA |
| | | GCTTTTCCTT GGTTACCCAT ACCGCGCCGG CGATGCGATT GACCCTGGAG |
| | | CGGGTACCCT TGAGCACGTC CCACATAATA TAGTCCACAA ATGGACTGGT |
| | | CTTGCTGATA AGCCTAGTGA GGACATGGGA AACTTCTATA CTGCCGGCAG |
| | | AGACCCCATA TTCTTCGGTC ACCACGCCAA TGTCGATCGG ATGTGGAATA |
| | | TATGGAAAAC TATAGGAGGT AAAAATAGAA AGGATTTCAC GGATACGGAT |
| | | TGGCTTGACG CCACGTTCGT CTTCTACGAC GAGAACAAAC AACTTGTTAA |
| | | AGTCAAGGTC TCGGACTGTG TCGACACTTC CAAGCTGAGA TACCAATATC |
| | | AGGATATTCC TATTCCATGG CTACCAAAAA AGACGAAGGC CAAAGCGAAG |
| | | ACGACCACCA AAAGTTCCAA GTCGGGAGTA GCGAAAGCGG CCGAACTCCC |
| | | AAAGACGACG ATCAGCAGCA TCGGAGACTT CCCAAAAGCT CTTAACTCAG |
| | | TGATAAGAGT AGAAGTTCCA AGGCCAAAGA AATCAAGAAG CAAGAAGGAG |
| | | AAAGAGGATG AGGAAGAGGT GTTACTGATA AAAGGAATAG AGCTAGATAG |
| | | AGAGAATTTC GTGAAGTTTG ATGTGTACAT CAACGACGAA GATTATTCAG |
| | | TGAGTAGGCC TAAGAATAGT GAGTTTGCAG GAAGCTTTGT GAACGTACCA |
| | | CACAAGCATA TGAAAGAAAT GAAGACGAAG ACCAATCTGA GGTTCGCGAT |
| | | AAATGAGCTG TTAGAGGACT TGGGAGCCGA AGATGATGAG AGTGTGATCG |
| | | TGACTATAGT CCCTCGTGCT GGGGGCGATG ATGTCACCAT TGGTGGAATT |
| | | GAGATCGAGT TTGTTTCCGA TTGA |
| 3 | Vitis | MASLPWSLTT FTAIANTTNI SAFPPSPLFQ RASHVPVARN RSRRFAPSKV |
| | vinifera | SCNSANGDPN SDSTSDVRET SSGKLDRRNV LLGIGGLYGA AGGLGATKPL |
| | PPO1 | AFGAPIQAPD ISKCGTATVP DGVTPTNCCP PVTTKIIDFQ LPSSGSPMRT |
| | amino acid | RPAAHLVSKE YLAKYKKAIE LQKALPDDDP RSFKQQANVH CTYCQGAYDQ |
| | sequence | VGYTDLELQV HASWLFLPFH RYYLYFNERI LAKLIDDPTI ALAYWAWDNP |
| | | DGMYMPTIYA SSPSSLYDEK RNAKHLPPTV IDLDYDGTEP TIPDDELKTD |
| | | NLAIMYKQIV SGATTPKLFL GYPYRAGDAI DPGAGTLEHV PHNIVHKWTG |
| | | LADKPSEDMG NFYTAGRDPI FFGHHANVDR MWNIWKTIGG KNRKDFTDTD |
| | | WLDATFVFYD ENKQLVKVKV SDCVDTSKLR YQYQDIPIPW LPKKTKAKAK |
| | | TTTKSSKSGV AKAAELPKTT ISSIGDFPKA LNSVIRVEVP RPKKSRSKKE |
| | | KEDEEEVLLI KGIELDRENF VKFDVYINDE DYSVSRPKNS EFAGSFVNVP |
| | | HKHMKEMKTK TNLRFAINEL LEDLGAEDDE SVIVTIVPRA GGDDVTIGGI |
| | | EIEFVSD |
| 4 | Vitis | GTCCTAGGCC TTTTTCATAT CCATATCGCC TGATCAGCAC CATGGCTTCT |
| | vinifera | CTTTCTCCTC CGGCCACCAC CACCACTACC AACATCTCCT CCCTGCAACC |
| | PPO2 | TTCTCCTTTG TTTCACAAAG CTTCTCAAGT CTCCCTAGTT GGAAAGCGAA |
| | nucleotide | ATCCTAGATT TGTGCGTAGA GTAGTATCGT GTAACGCTAC AAATGGTGAT |
| | sequence | GAAAGGTCAA CCTCTAGCTC CAAAAGTGGG GAATCTCCAC TAGGAAAGCT |
| | | TGATAGGAGG AATGTGCTTC TTGGCTTGGG AGGGCTTTAT GGTGTCTCCG |
| | | GTCTTGGTGC CGCTGATCCA TTCGCCTTTG CCGCCCCGAT ATCTCCACCG |
| | | GATCTAAGCG AGTGTGGGGC TGCAGACTTG CCTGCTGGTG CGCAGCCCAC |
| | | CAATTGTTGC CCGCCAGTGA GCAGCAAGAT CATAGATTTC AAGCTGCCTC |
| | | CTAAGAATTC CCCATTTCGC ATTAGGCCGG CGGCTCACTT GGCCAGCAAA |
| | | GAGTACATAG CCAAGTATAA AAAGGCCATT GAGCTCATGA AAGCTCTTCC |
| | | TGATGATGAT CCGCGGAGCT TCATGCAACA GGCCGATGTT CATTGCGCCT |
| | | ACTGCAATGG GGCCTACCAC CAAGTTGGGT TTCCCGATTT AGACCTCCAA |
| | | GTCCACAATT CATGGCTCTT CCTCCCATAC CACCGTTACT ATCTTTACTT |
| | | CTACGAGAAA ATCCTGGGAA AGTTGATTAA TGATCCCACC TTCGCTTTGC |
| | | CGTTTTGGAA CTGGGATGCT CCTGCTGGCA TGCAAATGCC TGCCATGTTT |
| | | GCTGACCCTA ACTCACCACT CTACGACAAG CTTCGCGACG CCCAAACATC |
| | | AGCCGCCAAA GCTGATCGAT CTCGACTACA CCTCACCGAC AGCAATGATA |
| | | CCAATGAGCA ACAAATTTCC AGCAATCTCT CCATTATGTA CCGGCAAATT |
| | | GTATCGAGTG CCAAGACTAC TAGTCTTTTC ATGGGAGCCG CTTATCGTGC |
| | | CGGCGACGAA CCTGACCCCG GCCAGGGTAC CCTTGAGAAC ATCCCACATG |
| | | GTCCAGTCCA CATATGGTGT GGTGACCGCA CCCAGCCGAA TATTGAGGAC |
| | | ATGGGCAACT TCTACTCTGC GGCCAGAGAC CCAATCTTCT TCTCTCACCA |
| | | TTCTAATGTG GACCGCATGT GGACGGTGTG GAAAGGGTTA GGAGGCAAGA |
| | | GAAAGGACTT CACCGACCCA GATTGGCTAG ACGCTGGGTT CGTCTTCTAC |
| | | GACGAGAATG CCCAACTTGT TCGCGTGAAG GTCCGCGACT GTCTCGACCC |
| | | TACCAAACTC AGGTATGCCT ATCAGGAGGT TGATATTCCA TGGCTGAAAT |
| | | CCCGTCCAAA GCCCCGTCAA ATATTGAAGA AGGTCGTCAA TAAATTGAAG |
| | | CACAAGTCTG GAGTAGCGAA TGCAGCCGAA ATTCCAAGTA GCAGCAGCAT |
| | | CGTCTTCCCC ATAAAATTGG ACTCAACGTT GAGAGTTGAT GTTCCAAGGC |
| | | CAAGGAAATC AAGGAGTAAG AAGGAGAAGG AGGATGAAGA AGAAGTGCTG |
| | | GTGATCAAAG GAATAGAGCT AGAAAGGGAT AAGTTCGTGA AGTTTGACAT |
| | | ATTCATCAAC GACGAAGACG ATCCAGTCAG CAGGCCTGAC AACACGGAGT |
| | | TTGCCGGAAG CTTTGTGAAC GTGCCACACC AACACTCCCA TGGGAAAAAG |
| | | AAGAACACGA TCCTCAGGAT AGGGATTAGT GAATTGTTGG AGGATTTGGA |
| | | AGCCGAAGAT GATGACAGTG TGGTGGTGAC TTTGGTCCCT CGTTATGGGG |
| | | CCGATGTTAT CACCATTGGC GGAATTGAAA TTGAGCTTGC CTCTTGATCA |
| | | CCTCACTACT CTTCATCAGA ATAATGGCGC CCTGATTTCC ATCCAGGATT |
| | | GCAATGATTT CCAGCTCTTT TTGTGTGTGC ATCAAATCGA ATTTCGTCGA |
| | | CACTTCCTCC CTTTATGTGA TTGGAATGTC TGTAATACCC CATTTTTGGA |
| | | TTTGAACTAT GGATTTCTTT GATCCACTGG GACATGATGT AATGGGGAAT |
| | | GAATAAATTC AACGTTATGT ATGGAACATT AGTATGCTTA ATTCGATAGC |
| | | CATGATTCTG CACTTTGTGT A |
| 5 | Vitis | ATGGCTTCTC TTTCTCCTCC GGCCACCACC ACCACTACCA ACATCTCCTC |
| | vinifera | CCTGCAACCT TCTCCTTTGT TTCACAAAGC TTCTCAAGTC TCCCTAGTTG |
| | PPO2 | GAAAGCGAAA TCCTAGATTT GTGCGTAGAG TAGTATCGTG TAACGCTACA |
| | coding | AATGGTGATG AAAGGTCAAC CTCTAGCTCC AAAAGTGGGG AATCTCCACT |
| | sequence | AGGAAAGCTT GATAGGAGGA ATGTGCTTCT TGGCTTGGGA GGGCTTTATG |
| | | GTGTCTCCGG TCTTGGTGCC GCTGATCCAT TCGCCTTTGC CGCCCCGATA |
| | | TCTCCACCGG ATCTAAGCGA GTGTGGGGCT GCAGACTTGC CTGCTGGTGC |
| | | GCAGCCCACC AATTGTTGCC CGCCAGTGAG CAGCAAGATC ATAGATTTCA |
| | | AGCTGCCTCC TAAGAATTCC CCATTTCGCA TTAGGCCGGC GGCTCACTTG |
| | | GCCAGCAAAG AGTACATAGC CAAGTATAAA AAGGCCATTG AGCTCATGAA |
| | | AGCTCTTCCT GATGATGATC CGCGGAGCTT CATGCAACAG GCCGATGTTC |
| | | ATTGCGCCTA CTGCAATGGG GCCTACCACC AAGTTGGGTT TCCCGATTTA |
| | | GACCTCCAAG TCCACAATTC ATGGCTCTTC CTCCCATACC ACCGTTACTA |
| | | TCTTTACTTC TACGAGAAAA TCCTGGGAAA GTTGATTAAT GATCCCACCT |
| | | TCGCTTTGCC GTTTTGGAAC TGGGATGCTC CTGCTGGCAT GCAAATGCCT |
| | | GCCATGTTTG CTGACCCTAA CTCACCACTC TACGACAAGC TTCGCGACGC |
| | | CCAAACATCA GCCGCCAAAG CTGATCGATC TCGACTACAC CTCACCGACA |
| | | GCAATGATAC CAATGAGCAA CAAATTTCCA GCAATCTCTC CATTATGTAC |
| | | CGGCAAATTG TATCGAGTGC CAAGACTACT AGTCTTTTCA TGGGAGCCGC |
| | | TTATCGTGCC GGCGACGAAC CTGACCCCGG CCAGGGTACC CTTGAGAACA |
| | | TCCCACATGG TCCAGTCCAC ATATGGTGTG GTGACCGCAC CCAGCCGAAT |
| | | ATTGAGGACA TGGGCAACTT CTACTCTGCG GCCAGAGACC CAATCTTCTT |
| | | CTCTCACCAT TCTAATGTGG ACCGCATGTG GACGGTGTGG AAAGGGTTAG |
| | | GAGGCAAGAG AAAGGACTTC ACCGACCCAG ATTGGCTAGA CGCTGGGTTC |
| | | GTCTTCTACG ACGAGAATGC CCAACTTGTT CGCGTGAAGG TCCGCGACTG |
| | | TCTCGACCCT ACCAAACTCA GGTATGCCTA TCAGGAGGTT GATATTCCAT |
| | | GGCTGAAATC CCGTCCAAAG CCCCGTCAAA TATTGAAGAA GGTCGTCAAT |
| | | AAATTGAAGC ACAAGTCTGG AGTAGCGAAT GCAGCCGAAA TTCCAAGTAG |
| | | CAGCAGCATC GTCTTCCCCA TAAAATTGGA CTCAACGTTG AGAGTTGATG |
| | | TTCCAAGGCC AAGGAAATCA AGGAGTAAGA AGGAGAAGGA GGATGAAGAA |
| | | GAAGTGCTGG TGATCAAAGG AATAGAGCTA GAAAGGGATA AGTTCGTGAA |
| | | GTTTGACATA TTCATCAACG ACGAAGACGA TCCAGTCAGC AGGCCTGACA |
| | | ACACGGAGTT TGCCGGAAGC TTTGTGAACG TGCCACACCA ACACTCCCAT |
| | | GGGAAAAAGA AGAACACGAT CCTCAGGATA GGGATTAGTG AATTGTTGGA |
| | | GGATTTGGAA GCCGAAGATG ATGACAGTGT GGTGGTGACT TTGGTCCCTC |
| | | GTTATGGGGC CGATGTTATC ACCATTGGCG GAATTGAAAT TGAGCTTGCC |
| | | TCTTGA |
| 6 | Vitis | MASLSPPATT TTTNISSLQP SPLFHKASQV SLVGKRNPRF VRRVVSCNAT |
| | vinifera | NGDERSTSSS KSGESPLGKL DRRNVLLGLG GLYGVSGLGA ADPFAFAAPI |
| | PPO2 | SPPDLSECGA ADLPAGAQPT NCCPPVSSKI IDFKLPPKNS PFRIRPAAHL |
| | amino acid | ASKEYIAKYK KAIELMKALP DDDPRSFMQQ ADVHCAYCNG AYHQVGFPDL |
| | sequence | DLQVHNSWLF LPYHRYYLYF YEKILGKLIN DPTFALPFWN WDAPAGMQMP |
| | | AMFADPNSPL YDKLRDAQTS AAKADRSRLH LTDSNDTNEQ QISSNLSIMY |
| | | RQIVSSAKTT SLFMGAAYRA GDEPDPGQGT LENIPHGPVH IWCGDRTQPN |
| | | IEDMGNFYSA ARDPIFFSHH SNVDRMWTVW KGLGGKRKDF TDPDWLDAGF |
| | | VFYDENAQLV RVKVRDCLDP TKLRYAYQEV DIPWLKSRPK PRQILKKVVN |
| | | KLKHKSGVAN AAEIPSSSSI VFPIKLDSTL RVDVPRPRKS RSKKEKEDEE |
| | | EVLVIKGIEL ERDKFVKFDI FINDEDDPVS RPDNTEFAGS FVNVPHQHSH |
| | | GKKKNTILRI GISELLEDLE AEDDDSVVVT LVPRYGADVI TIGGIEIELA |
| | | S |
| 7 | Vitis | TCATCACTTC TCCTCTAAAG CTATGGCTTC TTTGCCTTGG TCGCTCACAA |
| | vinifera | CCTCCACCGC CATCGCCAAC ACCACCAACA TTTCAGCCTT GCCACCTTCT |
| | PPO3 | CCCTTGTTTC ACAGGGTTTC TCATGTCCCC ATAGCCAGAA ACCGAAGCCG |
| | nucleotide | CAGATTTGCT CCTAGTAAGG TGTCGTGCAA TGCTGCGAAT GGTGATCCAA |
| | sequence | ACTCGGATTC TACCTCCGAC ATTCGAGAAA CTTCCCCAGG GAAGTTAGAT |
| | | AGGAGGAATG TGCTTCTTGG CATAGGAGGG CTGTATGGTG CTGCTGCCGG |
| | | TCTCGGCGCC ACTAAGCCAT TAGCCTTTGG GGCTCCCATC CAGGCACCGG |
| | | ATATATCCAA GTGTGGTACC GCCACCGTGC CTGATGGTGT AACGCCCACA |
| | | ATTGTTGCCC GCCAGTCACC ACAAAGATTA TAGATTTCCA GCTACCTTCC |
| | | TCAGGTTCCC CCATGCGTAC CAGGCCAGCT GCTCACTTGG TCAGCAAAGA |
| | | GTACTTAGCC AAGTATAAAA AGGCCATTGA GCTGCAGAAA GCTCTTCCTG |
| | | ATGATGATCC GCGTAGTTTC AAGCAACAGG CTAATGTCCA TTGCACCTAT |
| | | TGCCAAGGGG CTTATGATCA GGTTGGGTAT ACCGACCTAG AACTCCAGGT |
| | | TCATGCTTCA TGGCTTTTCC TCCCTTTCCA CCGTTACTAT CTCTACTTCA |
| | | ATGAGAGAAT TCTTGCAAAA TTGATCGACG ATCCCACCTT CGCTTTGCCC |
| | | TATTGGGCTT GGGATAACCC TGATGGCATG TATATGCCGG CCATCTATGC |
| | | TAGTTCCCCA TCATCACTTT ACGACGAGAA GCGCAACGCC AAGCACCTGC |
| | | CTCCGACTGT GATCGATCTC GACTACGATG GCACCGAACC CACAATCCCT |
| | | GATGACGAAC TAAAAGCCGA CAATCTGGCA ATCATGTACA AACAAATTGT |
| | | GTCGGGTGCC ACGACTCCTA AGCTTTTCTT TGGTTACCCA TACCGCGCCG |
| | | GCGATGCGAT TGACCCTGGA GCGGGTACCC TTGAGCTTGT CCCACATAAT |
| | | ATAGTCCACA AATGGACTGG TCTTGCTGAT AAGCCTAGTG AGGACATGGG |
| | | AAACTTCTAT ACTGCCGGCA GAGACCCCAT ATTCTTCGGT CACACGCCAA |
| | | TGTCGATCGA ATGTGGAATA TATGGAAAAC TATAGGAAGG AAAAATAAAA |
| | | AGGATTTCAC GGGATCCGGA TGGTCTGGAC GCCACTTTGG TCTTTTACGA |
| | | CGGGAACAAA CAATTGGTAG AAGTGAAGGT CTCGGAGTGG TTCGACACTT |
| | | CCAAGCTGAG ATACCAATAT CAGGATATTC TTCTTCCATG GCTACCAAAA |
| | | AATACGAAGG CCAAAGCGAA GACGACCACC AAAAGTTCCA AGTCGGGAGT |
| | | AGCGAAAGCG GCCGAACTCC CAAAGACGAC GATCAGCAGC ATCGGAGACT |
| | | TCCCAAAAGC TCTTAACTCA GTGATAAGAG TAGAAGTTCC AAGGCCAAAG |
| | | AAATCAAGAA GCAAGAAGGA GAAAGAGGAC GAGGAAGAGG TGTTACTGAT |
| | | AAAAGGAATA GAGCTAGATA GAGAGAATTT CGTGAAGTTT GATGTGTACA |
| | | TCAACGACGA AGATTATTCA GTGAGTAGGC CTAAGAATAG TGAGTTTGCA |
| | | GGAAGCTTTG TGAACGTACC ACACAAGCAT ATGAAAGAAA TGAAGACGAA |
| | | GACCAATCTG AGGTTGGCGA TAAATGAGCT GTTAGAGGAC TTGGGAGCCG |
| | | AAGATGATGA GAGTGTGATC GTGACTATAG TCCCTCGTGC TGGGGGCGAT |
| | | GATGTCACCA TTGGTGGAAT TGAGATCGAG TTTGTTTCCG ATTGATCCCA |
| | | CCTTTCAATG ATTATCCATT ATATGTATGT ATCAGGTAAG TCACATCTTT |
| | | ATGTGACTAA TGGAAAATGT GAGACTTCTC TGTACTCTCC CGTCAAGTCT |
| | | TTTATTAATT TAGAGCTGTT TGGTTTAAAT TTAA |
| 8 | Vitis | ATGCGTACCA GGCCAGCTGC TCACTTGGTC AGCAAAGAGT ACTTAGCCAA |
| | vinifera | GTATAAAAAG GCCATTGAGC TGCAGAAAGC TCTTCCTGAT GATGATCCGC |
| | PPO3 | GTAGTTTCAA GCAACAGGCT AATGTCCATT GCACCTATTG CCAAGGGGCT |
| | coding | TATGATCAGG TTGGGTATAC CGACCTAGAA CTCCAGGTTC ATGCTTCATG |
| | sequence | GCTTTTCCTC CCTTTCCACC GTTACTATCT CTACTTCAAT GAGAGAATTC |
| | | TTGCAAAATT GATCGACGAT CCCACCTTCG CTTTGCCCTA TTGGGCTTGG |
| | | GATAACCCTG ATGGCATGTA TATGCCGGCC ATCTATGCTA GTTCCCCATC |
| | | ATCACTTTAC GACGAGAAGC GCAACGCCAA GCACCTGCCT CCGACTGTGA |
| | | TCGATCTCGA CTACGATGGC ACCGAACCCA CAATCCCTGA TGACGAACTA |
| | | AAAGCCGACA ATCTGGCAAT CATGTACAAA CAAATTGTGT CGGGTGCCAC |
| | | GACTCCTAAG CTTTTCTTTG GTTACCCATA CCGCGCCGGC GATGCGATTG |
| | | ACCCTGGAGC GGGTACCCTT GAGCTTGTCC CACATAATAT AGTCCACAAA |
| | | TGGACTGGTC TTGCTGATAA GCCTAGTGAG GACATGGGAA ACTTCTATAC |
| | | TGCCGGCAGA GACCCCATAT TCTTCGGTCA CACGCCAATG TCGATCGAAT |
| | | GTGGAATATA TGGAAAACTA TAG |
| 9 | Vitis | MRTRPAAHLV SKEYLAKYKK AIELQKALPD DDPRSFKQQA NVHCTYCQGA |
| | vinifera | YDQVGYTDLE LQVHASWLFL PFHRYYLYFN ERILAKLIDD PTFALPYWAW |
| | PPO3 | DNPDGMYMPA IYASSPSSLY DEKRNAKHLP PTVIDLDYDG TEPTIPDDEL |
| | amino acid | KADNLAIMYK QIVSGATTPK LFFGYPYRAG DAIDPGAGTL ELVPHNIVHK |
| | sequence | WTGLADKPSE DMGNFYTAGR DPIFFGHTPM SIECGIYGKL |
| 10 | Vitis | GACCGAGGCC TTAGCGCGTT ATCACTCATC ACTTCTCCTC TAAAGCTATG |
| | vinifera | GCTTCTTTGC CTTGGTCGCT CACAACCTCC ACCGCCATCG CCAACACCAC |
| | PPO4 | CAACATTTCA GCCTTGCCAC CTTCTCCCTT GTTTCACAGG GTTTCTCATG |
| | nucleotide | TCCCCATAGC CAGAAACCGA AGCCGCAGAT TTGCTCCTAG TAAGGTGTCG |
| | sequence | TGCAATGCTG CGAATGGTGA TCCAAACTCG GATTCTACCT CCGACATTCG |
| | | AGAAACTTCC CCAGGGAAGT TAGATAGGAG GAATGTGCTT CTTGGCATAG |
| | | GAGGGCTGTA TGGTGCTGCT GCCGGTCTCG GCGCCACTAA GCCATTAGCC |
| | | TTTGGGGCTC CCATCCAGGC ACCGGATATA CGCAAGTGTG GTACCGCCAC |
| | | CGTGCCTAAT GGTGTAATTC CCACAAATTG TTGCCCGCCA GTCACCACAA |
| | | AGCCTATACC TTTCCAGCTA CCTTCCTCAG ATTCCCCCAT GCGTACCAGG |
| | | CCAGCTGCTC ACTTGGTCAG CAAAGAGTAC TTAGCCAAGT ATAAAAAGGC |
| | | CATTGAGCTG CAGAAAGCTC TTCCTGATGA TGATCCGCGT AGTTTCAAGC |
| | | AACAGGCCAA TGTCCATTGC GCCTATTGCC AAGGGGCTTA TGATCAGGTT |
| | | GGGTATACCG ACCTAGAACT CCAGGTTCAT GCTTCATGGG TTTCTCATGT |
| | | CCCCATAGCC AGAAACCGAA GCCGCAGATT TGCTCCTAGT AAGGTGTCGT |
| | | GCAATGCTGC GAATGGTGAT CCAAACTCGG ATTCTACCTC CGACATTCGA |
| | | GAAACTTCCC CAGGGAAGTT AGATAGGAGG AATGTGCTTC TTGGCATAGG |
| | | AGGGCTGTAT GGTGCTGCTG CCGGTCTCGG CGCCACTAAG CCATTAGCCT |
| | | TTGGGGCTCC CATCCAGGCA CCGGATATAT CCAAGTGTGG TACCGCCACC |
| | | GTGCCTGATG GTGTAACGCC CACAATTGTT GCCCGCCAGC CAGCTGCTCA |
| | | CTTGGTCAGC AAAGAGTACT TAGCCAAGTA TAAAAAGGCC ATTGAGCTGC |
| | | AGAAAGCTCT TCCTGATGAT GATCCGCGTA GTTTCAAGCA ACAGGCTAAT |
| | | GTCCATTGCA CCTATTGCCA AGGGGCTTAT GATCAGGTTG GGTATACCGA |
| | | CCTAGAACTC CAGAAAGTCA AGGTCTCGGA CTGTGTCGAC ACTTCCAAGC |
| | | TGAGATACCA ATATCAGGAT ATTNCAATCC CATGGCTACC AAAAAATACG |
| | | AAGGCCAAAG CGAAGACGAC CACCAAAAGT TCCAAGTCGG GAGTAGCGAA |
| | | AGCGGCCGAA CTCCCAAAGA CGACGATCAG CAGCATCGGA GACTTCCCAA |
| | | AAGCTCTTAA CTCAGTGATA AGAGTAGAAG TTCCAAGGCC AAAGAAATCA |
| | | AGAAGCAAGA AGGAGAAAGA GGACGAGGAA GAGGTGTTAC TGATAAAAGG |
| | | AATAGAGCTA GATAGAGAGA ATTTCGTGAA GTTTGATGTG TACATCAACG |
| | | ACGAAGATTA TTCAGTGAGT AGGCCTAAGA ATAGTGAGTT TGCAGGAAGC |
| | | TTTGTGAACG TACCACACAA GCATATGAAA GAAATGAAGA CGAAGACCAA |
| | | TCTGAGGTTG GCGATAAATG AGCTGTTAGA GGACTTGGGA GCCGAAGATG |
| | | ATGAGAGTGT GATCGTGACT ATAGTCCCTC GTGCTGGGGG CGATGATGTC |
| | | ACCATTGGTG GAATTGAGAT CGAGTTTGTT TCCGATTGAT CCCACCTTTC |
| | | AATGATTATC CATTATATGT ATGTATCAGG TAAGTCACAT CTTTATGTGA |
| | | CTAATGGAAA ATGTGAGACT TCTCTGTACT CTCCCGTCAA GTCTTTTATT |
| | | AATTTAGAGC TGTTTGGTTT AAATTTAA |
| 11 | Vitis | ATGGCTTCTT TGCCTTGGTC GCTCACAACC TCCACCGCCA TCGCCAACAC |
| | vinifera | CACCAACATT TCAGCCTTGC CACCTTCTCC CTTGTTTCAC AGGGTTTCTC |
| | PPO4 | ATGTCCCCAT AGCCAGAAAC CGAAGCCGCA GATTTGCTCC TAGTAAGGTG |
| | coding | TCGTGCAATG CTGCGAATGG TGATCCAAAC TCGGATTCTA CCTCCGACAT |
| | sequence | TCGAGAAACT TCCCCAGGGA AGTTAGATAG GAGGAATGTG CTTCTTGGCA |
| | | TAGGAGGGCT GTATGGTGCT GCTGCCGGTC TCGGCGCCAC TAAGCCATTA |
| | | GCCTTTGGGG CTCCCATCCA GGCACCGGAT ATACGCAAGT GTGGTACCGC |
| | | CACCGTGCCT AATGGTGTAA TTCCCACAAA TTGTTGCCCG CCAGTCACCA |
| | | CAAAGCCTAT ACCTTTCCAG CTACCTTCCT CAGATTCCCC CATGCGTACC |
| | | AGGCCAGCTG CTCACTTGGT CAGCAAAGAG TACTTAGCCA AGTATAAAAA |
| | | GGCCATTGAG CTGCAGAAAG CTCTTCCTGA TGATGATCCG CGTAGTTTCA |
| | | AGCAACAGGC CAATGTCCAT TGCGCCTATT GCCAAGGGGC TTATGATCAG |
| | | GTTGGGTATA CCGACCTAGA ACTCCAGGTT CATGCTTCAT GGGTTTCTCA |
| | | TGTCCCCATA GCCAGAAACC GAAGCCGCAG ATTTGCTCCT AGTAAGGTGT |
| | | CGTGCAATGC TGCGAATGGT GATCCAAACT CGGATTCTAC CTCCGACATT |
| | | CGAGAAACTT CCCCAGGGAA GTTAGATAGG AGGAATGTGC TTCTTGGCAT |
| | | AGGAGGGCTG TATGGTGCTG CTGCCGGTCT CGGCGCCACT AAGCCATTAG |
| | | CCTTTGGGGC TCCCATCCAG GCACCGGATA TATCCAAGTG TGGTACCGCC |
| | | ACCGTGCCTG ATGGTGTAAC GCCCACAATT GTTGCCCGCC AGCCAGCTGC |
| | | TCACTTGGTC AGCAAAGAGT ACTTAGCCAA GTATAAAAAG GCCATTGAGC |
| | | TGCAGAAAGC TCTTCCTGAT GATGATCCGC GTAGTTTCAA GCAACAGGCT |
| | | AATGTCCATT GCACCTATTG CCAAGGGGCT TATGATCAGG TTGGGTATAC |
| | | CGACCTAGAA CTCCAGAAAG TCAAGGTCTC GGACTGTGTC GACACTTCCA |
| | | AGCTGAGATA CCAATATCAG GATATTNCAA TCCCATGGCT ACCAAAAAAT |
| | | ACGAAGGCCA AAGCGAAGAC GACCACCAAA AGTTCCAAGT CGGGAGTAGC |
| | | GAAAGCGGCC GAACTCCCAA AGACGACGAT CAGCAGCATC GGAGACTTCC |
| | | CAAAAGCTCT TAACTCAGTG ATAAGAGTAG AAGTTCCAAG GCCAAAGAAA |
| | | TCAAGAAGCA AGAAGGAGAA AGAGGACGAG GAAGAGGTGT TACTGATAAA |
| | | AGGAATAGAG CTAGATAGAG AGAATTTCGT GAAGTTTGAT GTGTACATCA |
| | | ACGACGAAGA TTATTCAGTG AGTAGGCCTA AGAATAGTGA GTTTGCAGGA |
| | | AGCTTTGTGA ACGTACCACA CAAGCATATG AAAGAAATGA AGACGAAGAC |
| | | CAATCTGAGG TTGGCGATAA ATGAGCTGTT AGAGGACTTG GGAGCCGAAG |
| | | ATGATGAGAG TGTGATCGTG ACTATAGTCC CTCGTGCTGG GGGCGATGAT |
| | | GTCACCATTG GTGGAATTGA GATCGAGTTT GTTTCCGATT GA |
| 12 | Vitis | MASLPWSLTT STAIANTTNI SALPPSPLFH RVSHVPIARN RSRRFAPSKV |
| | vinifera | SCNAANGDPN SDSTSDIRET SPGKLDRRNV LLGIGGLYGA AAGLGATKPL |
| | PPO4 | AFGAPIQAPD IRKCGTATVP NGVIPTNCCP PVTTKPIPFQ LPSSDSPMRT |
| | amino acid | RPAAHLVSKE YLAKYKKAIE LQKALPDDDP RSFKQQANVH CAYCQGAYDQ |
| | sequence | VGYTDLELQV HASWVSHVPI ARNRSRRFAP SKVSCNAANG DPNSDSTSDI |
| | | RETSPGKLDR RNVLLGIGGL YGAAAGLGAT KPLAFGAPIQ APDISKCGTA |
| | | TVPDGVTPTI VARQPAAHLV SKEYLAKYKK AIELQKALPD DDPRSFKQQA |
| | | NVHCTYCQGA YDQVGYTDLE LQKVKVSDCV DTSKLRYQYQ DIIPWLPKNT |
| | | KAKAKTTTKS SKSGVAKAAE LPKTTISSIG DFPKALNSVI RVEVPRPKKS |
| | | RSKKEKEDEE EVLLIKGIEL DRENFVKEDV YINDEDYSVS RPKNSEFAGS |
| | | FVNVPHKHMK EMKTKTNLRL AINELLEDLG AEDDESVIVT IVPRAGGDDV |
| | | TIGGIEIEFV SD |
| 13 | Vitis | ATGGATAATG GGAGATTTCC ATGGGGGGCC TATACAGTGC AGAAAATCAG |
| | vinifera | TTTTCCTTGT CCTTTTCAGC TTTTTATTTA TTTACTTTTC TACGG |
| | PPO5 | CAAGTGTTTT CTCGACATCC CTAAGGTAAG AATACGTACT ACCTACCCCA |
| | nucleotide | GTTCATTCTG TACAGAGACT GAGGCCATAG AGCGGTCTCA CTTATCACCC |
| | sequence | GTAACTCCTC TAAAGCTATG GCTTCTTTGC CTTGGCAGCT CACAACCTCC |
| | | ACCGCCACCG CCACCGCCAC CGTCACCACC TCCAACATTT CGGCCTTGTC |
| | | ACCTTCTCCC TTGTTTCACA GGGTTTCTCG GGGGAGTCAG AAATCGAAAC |
| | | CACAGATCTG CTCCTAGTAA AGTGTCGTGC AATTCTACAA ATGGTGATCC |
| | | AAACTCGGAT TCTACCTCCG ACATTCGAGA AACTTCCCCA GGGAAGTTAG |
| | | ATAGGAGGAA TGTGCTTCTT GGCATAGGAG GGCTGTATGG TGCCGCTGGC |
| | | GGCCTCGGCG CCACTGAGCC ATTAGCCTTT GGGGCTCCCA TCCAGGCACC |
| | | GGATGTAAAC AAGTGTGGTG CCGCCACCGT GCCTGAGGGT GTAACGCCCA |
| | | CAAATTGTTG CCCGCCAGCC ACCACAAAGA TTATAGATTT CCAGCTACCT |
| | | TCCCCAGATT CCCCCATGCG CATTAGGCCA GCTGCTCACT TGGTCAGCAA |
| | | AGAGTACATA GCCAAGTATA AAAAGGCCAT TGAGCTAATG AAAGCTCTTC |
| | | CTGATGATGA TCCACGGAGC TTCATGCAGC AGGCCAATGT CCATTGCGCC |
| | | TATTGCGATG GGGCTTATGA TCAGGTTGGG TTTCCGGACC TAGAGCTCCA |
| | | GGTTCATTTT TCATGGCTTT TCTTCCCTTT CCACCGTTTC TATTGCTACT |
| | | TTCATGAGAG AATTCTTGCA AAACTGATTA ACGATCCCAC CTTCGCTTTG |
| | | CCCTATTGGA GCTGGGATTC CCCTCCTCCT GGAATGCAAA TGCCGGGCAT |
| | | CTATGCTGAT TCCACATCAA CACTCTACGA CGCGAAGCGC AACGCCAACC |
| | | ACCTGCCTCC GACTGTAATC GATCTCGACT ACGATTTCAC CGAACCCACA |
| | | ATCCCAGTTT CGGAATTAGT TGCCAACAAC CTGTCAATCA TGTACCGGCA |
| | | AGTTGTGTCG GGTGCCACGA CTGCTAAGCT ATTCTTTGGT GCCCCATACC |
| | | GTGCTGGCGA TCAGCCTGAC CCCGGACCGG GTACCATTGA GAACGTCCCA |
| | | CATGGTCCAG TCCACATATG GACTGGACTT TTTGACGAGC CTCATGAGGA |
| | | CATGGGAAAC TTCTATACTG CCGGCAGAGA CCCCATATTT TTCGGTCACC |
| | | ACTCCAACGT CGACCGTATG TGGACGCTAT GGAAAGCTAT AGGAGGTAAA |
| | | AATAGAACGG AATTCACGGA CCCAGATTGG CTAAACGCCG GGTTCGTCTT |
| | | CTACGACGAG AACGCCCAAC TTGTTCGCGT CAAGGTCCGA GACTGCCTCG |
| | | ACCATCTCAA GCTGAGATAC AAATATCAGG ATATTCCTAT TCCATGGCTA |
| | | AAAAAAAGTA CGCAGCCCAA AGCGAAGACG ACCACCAAAA GTTCCAAGTC |
| | | GGGAGTAGCG AAAGCGGCGG AACTCCCAAA GACGACGATC AGCAGCAGTG |
| | | GAGGCTTCCC AAAACCTCTT AACTCAGTAA TAAGAGTAGA AGTTCCGAGG |
| | | CCAAAGAAAT CAAGAAGCAA GAAGGAGAAA GAGGATGAAG AAGAGGTGTT |
| | | GCTGATAAAA GGAATAGAGC TAGATAGAGA TGATTTCGTG AAGTTTGATG |
| | | TGTACATCAA CGACGAAGAT TATTCAGTGA GTAAGCCTAG TAATAGTGAG |
| | | TTCGCAGGAA GCTTGGTGAA CGTACCACAC AAGCATACGC AGAGAATGAA |
| | | GACAAAGACC AGCCTGAGGT TAGCGATAAA TGAATTACTA GATAACTTGG |
| | | GGGCTGAAGA TGATGAGAGT GTGATCGTGA CTATAGTCCC TCGTGCTGGG |
| | | ACCGATGGTG TCACCATTGG TGGAATTGAG ATCGAGTTTG TTTCCGATTG |
| | | ATCCCACCTT TCGATTCTTA TCCATTATAT GTATCCGGTC ACGTGTGTTT |
| | | CGAAATCAAT GAAGGTCATT TCTACGCATT ATGTATCATG TAAATCACAT |
| | | CAGTATGTGA CTAATGGAAA ATGTGAAATT TCTTTCTACT TTCCCCTTCC |
| 14 | Vitis | ATGGATAATG GGAGATTTCC ATGGGGGGCC TATACAGTGC AGAAAATCAG |
| | vinifera | TTTTCCTTGT CCTTTTCAGC TTTTTATTTA TTTACTTTTC TACGGCAAGT |
| | PPO5 | GTTTTCTCGA CATCCCTAAG GTAAGAATAC GTACTACCTA CCCCAGTTCA |
| | coding | TTCTGTACAG AGACTGAGGC CATAGAGCGG TCTCACTTAT CACCCGTAAC |
| | sequence | TCCTCTAAAG CTATGGCTTC TTTGCCTTGG CAGCTCACAA CCTCCACCGC |
| | | CACCGCCACC GCCACCGTCA CCACCTCCAA CATTTCGGCC TTGTCACCTT |
| | | CTCCCTTGTT TCACAGGGTT TCTCGGGGGA GTCAGAAATC GAAACCACAG |
| | | ATCTGCTCCT AGTAAAGTGT CGTGCAATTC TACAAATGGT GATCCAAACT |
| | | CGGATTCTAC CTCCGACATT CGAGAAACTT CCCCAGGGAA GTTAGATAGG |
| | | AGGAATGTGC TTCTTGGCAT AGGAGGGCTG TATGGTGCCG CTGGCGGCCT |
| | | CGGCGCCACT GAGCCATTAG CCTTTGGGGC TCCCATCCAG GCACCGGATG |
| | | TAAACAAGTG TGGTGCCGCC ACCGTGCCTG AGGGTGTAAC GCCCACAAAT |
| | | TGTTGCCCGC CAGCCACCAC AAAGATTATA GATTTCCAGC TACCTTCCCC |
| | | AGATTCCCCC ATGCGCATTA GGCCAGCTGC TCACTTGGTC AGCAAAGAGT |
| | | ACATAGCCAA GTATAAAAAG GCCATTGAGC TAATGAAAGC TCTTCCTGAT |
| | | GATGATCCAC GGAGCTTCAT GCAGCAGGCC AATGTCCATT GCGCCTATTG |
| | | CGATGGGGCT TATGATCAGG TTGGGTTTCC GGACCTAGAG CTCCAGGTTC |
| | | ATTTTTCATG GCTTTTCTTC CCTTTCCACC GTTTCTATTG CTACTTTCAT |
| | | GAGAGAATTC TTGCAAAACT GATTAACGAT CCCACCTTCG CTTTGCCCTA |
| | | TTGGAGCTGG GATTCCCCTC CTCCTGGAAT GCAAATGCCG GGCATCTATG |
| | | CTGATTCCAC ATCAACACTC TACGACGCGA AGCGCAACGC CAACCACCTG |
| | | CCTCCGACTG TAATCGATCT CGACTACGAT TTCACCGAAC CCACAATCCC |
| | | AGTTTCGGAA TTAGTTGCCA ACAACCTGTC AATCATGTAC CGGCAAGTTG |
| | | TGTCGGGTGC CACGACTGCT AAGCTATTCT TTGGTGCCCC ATACCGTGCT |
| | | GGCGATCAGC CTGACCCCGG ACCGGGTACC ATTGAGAACG TCCCACATGG |
| | | TCCAGTCCAC ATATGGACTG GACTTTTTGA CGAGCCTCAT GAGGACATGG |
| | | GAAACTTCTA TACTGCCGGC AGAGACCCCA TATTTTTCGG TCACCACTCC |
| | | AACGTCGACC GTATGTGGAC GCTATGGAAA GCTATAGGAG GTAAAAATAG |
| | | AACGGAATTC ACGGACCCAG ATTGGCTAAA CGCCGGGTTC GTCTTCTACG |
| | | ACGAGAACGC CCAACTTGTT CGCGTCAAGG TCCGAGACTG CCTCGACCAT |
| | | CTCAAGCTGA GATACAAATA TCAGGATATT CCTATTCCAT GGCTAAAAAA |
| | | AAGTACGCAG CCCAAAGCGA AGACGACCAC CAAAAGTTCC AAGTCGGGAG |
| | | TAGCGAAAGC GGCGGAACTC CCAAAGACGA CGATCAGCAG CAGTGGAGGC |
| | | TTCCCAAAAC CTCTTAACTC AGTAATAAGA GTAGAAGTTC CGAGGCCAAA |
| | | GAAATCAAGA AGCAAGAAGG AGAAAGAGGA TGAAGAAGAG GTGTTGCTGA |
| | | TAAAAGGAAT AGAGCTAGAT AGAGATGATT TCGTGAAGTT TGATGTGTAC |
| | | ATCAACGACG AAGATTATTC AGTGAGTAAG CCTAGTAATA GTGAGTTCGC |
| | | AGGAAGCTTG GTGAACGTAC CACACAAGCA TACGCAGAGA ATGAAGACAA |
| | | AGACCAGCCT GAGGTTAGCG ATAAATGAAT TACTAGATAA CTTGGGGGCT |
| | | GAAGATGATG AGAGTGTGAT CGTGACTATA GTCCCTCGTG CTGGGACCGA |
| | | TGGTGTCACC ATTGGTGGAA TTGAGATCGA GTTTGTTTCC GATTGA |
| 15 | Vitis | MDNGRFPWGA YTVQKISFPC PFQLFIYLLF YGKCFLDIPK VRIRTTYPSS |
| | vinifera | FCTETEAIER SHLSPVTPLK LWLLCLGSSQ PPPPPPPPPS PPPTERPCHL |
| | PPO5 | LPCFTGFLGG VRNRNHRSAP SKVSCNSTNG DPNSDSTSDI RETSPGKLDR |
| | amino acid | RNVLLGIGGL YGAAGGLGAT EPLAFGAPIQ APDVNKCGAA TVPEGVTPTN |
| | sequence | CCPPATTKII DFQLPSPDSP MRIRPAAHLV SKEYIAKYKK AIELMKALPD |
| | | DDPRSFMQQA NVHCAYCDGA YDQVGFPDLE LQVHESWLFF PFHRFYCYFH |
| | | ERILAKLIND PTFALPYWSW DSPPPGMQMP GIYADSTSTL YDAKRNANHL |
| | | PPTVIDLDYD FTEPTIPVSE LVANNLSIMY RQVVSGATTA KLFFGAPYRA |
| | | GDQPDPGPGT IENVPHGPVH IWTGLEDEPH EDMGNFYTAG RDPIFFGHHS |
| | | NVDRMWTLWK AIGGKNRTEF TDPDWLNAGF VFYDENAQLV RVKVRDCLDH |
| | | LKLRYKYQDI PIPWLKKSTQ PKAKTTTKSS KSGVAKAAEL PKTTISSSGG |
| | | FPKPLNSVIR VEVPRPKKSR SKKEKEDEEE VLLIKGIELD RDDFVKEDVY |
| | | INDEDYSVSK PSNSEFAGSL VNVPHKHTQR MKTKTSLRLA INELLDNLGA |
| | | EDDESVIVTI VPRAGTDGVT IGGIEIEFVS D |
| 16 | Vitis | ATGGGAAAAT CATCTCTATT TCCTTTGAAT TTTTTTTTAA AAAAATTAGT |
| | vinifera | TTGGAATTTC AATTTTTTGT AATATTAATT TTTTCTTTTA ATTTGGGATT |
| | PPO6 | TTTTAAAAAA AAAATTATTT TTTATAATAT TAATTTTAAA AAAACTTTTG |
| | nucleotide | AATTTTTTTT TAAATGGGAA AATAGTCTCT TGAAGAGAAA ATTTCCCTTT |
| | sequence | TTTAAATTAG TTTCCATTTT TATGTAATAT TAAGGGATTT TTTTTAATTT |
| | | TTTTTTAAAT TATTTTTTAT AATATTAATT TTTTTTAAAA AAAATTCAAA |
| | | GGGAAATCGT CTCTTGAAGA GATGATTTCC CTTTAATATT AATTTTGGAT |
| | | TTTGTTTTTT TTTTTTTTAA ATTTAATTAT TTTTTATAAT ATTAATTTTA |
| | | AATTTTTTTT TTAAATTAGT TTGGAAAATA TATATTTTTT TTTTAAATGG |
| | | TAAATTAAAT TTTTTTTTTT AAGGGAAATT GTCTCTTCAA GAGGAAATTT |
| | | ATTTATTTTT TAAAATGGTA AATTAAATTT TTTTTAAGGC AAGAGACGAT |
| | | TTCCCTTTTG GAAATTTTTT TTTTCTTTTT TTTAAATTAG TTTGAAAAAT |
| | | ATATATATTT TTTAAAATGG TAAATTAAAA TTTTTTTAAA GGGAAATCAT |
| | | CTCTTGAAGA GACAGATTTC CCTTTTGGAT TTTTTTTTTT TTTAAATTAG |
| | | TTTGGAAAAT TTTTTTTTTA AATGGTAAAT TTAAAAAAAA TTTAAGGAAA |
| | | TTTTTTTTTT TTTTTAAATG GTAAATTTAA AATTTTTTTT AAGGGAAATC |
| | | GTCATTTCAA GAGATGATTT CCCTTTTGGA ATTTTTTTTT TTTAAATTAG |
| | | TTTGGAAAAT ATATATATTT TTTAAAATGG TAAATTAAAT TTTTTTTTTT |
| | | TTTAAGGGAA ATCGGCTCTT GAAGAGACGA TTTCCCTTTT GGAATTTTTT |
| | | TTTAAAAATT AGTTTGGAAA ATATATATAT TTTTTTAAAT GGTAAATTAA |
| | | AATATTTTTA AAGGAAAATC GTCTCTTCAA GAGACACTTT TGGAATTTTT |
| | | TTTTGTTTTT TAAATTAGTT TGGAATTTTT TTTAAATGGT AAATTAAAAT |
| | | TTTTTTAAGG GAATTTTGGA TTTTTTTTTT TTTAAATGAA GAGAAGATTT |
| | | CCCTTTGAAT TTCTTTTTAT TTCCATTTAT GATTTTAAAA CCGTCTTTTT |
| | | TAAAGACGAT TTTGGTTTTA AAAAAAAAAA ATTTTAGCGT GCCTCTCTCT |
| | | CCTACCTACA AAATGTTCTT GGAATAACTT TTTCTACTAA GACAATTTAA |
| | | GAATAATTTT TTTTAATTAC CGTTATCTTG TGAAAAGTCC CCGCCACCAC |
| | | CACCAACATT TCGGCCTTGT CACCTTCCCC CTTGTTTCTC AGGGTTTCTC |
| | | ATGTCCCCAT CGTCAGAAAT CGAAACCACA GATCTGCTCC AAGTAAAATG |
| | | ACTTGCAATT CTGCAAATGG TGATCCAAAC TCGGATTCCA CCTCCGACAT |
| | | TCGAGGAACT ATGCCAAGAA GCACATTCCT CCTATCTAAC TTCCCTTCCC |
| | | TAGGGAAGTT AGATTGGAGG AATGTGCTTC TTGGCATAGG AGGGCTGTAT |
| | | GGTGCCGCTG GCAGCCTCGC GCCACTGAGC CATTAGCCTT CGGAGCTCCC |
| | | ATCCAGGCAC CGGATGTAAC TAAGTGTGGT GTCGCCACCG TGCCTGCGGG |
| | | TGTAACGGCC ACAAATTGTT GCCCGCCAGC CACCACAAAG ATTATAGATT |
| | | TCCAGCTGCC TTCCCCAGAT TCCCTCCTGC GCATTAGGCC AGCTGCTCAC |
| | | TTGGTCATCA AAGAGTACAG AGCCAAGTAT AAAAAGGCCA TTCAGCTAAT |
| | | GAAAGCTCTT CCTGATGATG ATCCATGGAG CTTCAGGCAA CAGGCCAGTG |
| | | TCCGTTGCGC CTATTGCAAT GGGGCTGATG ATCAGGTTGG GTTTCCGGAT |
| | | CTAGAAATCC AGGTTCATGC TTCATGGCTT TTCTTCCCTT TCCACCGTTT |
| | | ATATGCCTGC TTCCATGAGA GAATTCTTGC AGAACTGATT AACGATCCCA |
| | | CCTCCGCTTT GCCCTATTGG AGCTGGGATT CCCCTCCTGG TATGCAAATG |
| | | CCGGCCATCT ATGCTGCTTC CACATCAACA CGCTACGACG CGAAGCGCAA |
| | | CGCCAAACAC CTGCCTCCGA CTGTAATCAA TCTTGACTAC GATTTCACCG |
| | | AATCCACATC CCCGATTTGG AAATAGTTGC CAACAATCTG TCAATCGTAT |
| | | ACCGGCAAGT TGTGTCGGGT GCCACGACTG CTAAGCTTTT CTTTGGTAGC |
| | | CCATACCGTG CCGGAGATCA GCCTGACCCT GGAGAGGGTA CCATTGATAG |
| | | CGTCCCGGCC ACATGGTCCA GTCCACATAT GGACTGGTCT TTTTGACAAG |
| | | CCTCATGAGG ACATGGGAAA CTTCTATACT GCTGGCAAAG ACCCCATATT |
| | | CTTCGGTCAC CACGCCAACG TCGACCGTAT GTGGATGCTA TGGAAAGCTA |
| | | TAGGAGGTAA AAATAGAACT GAATTCACGG ACCCAGATTC GCTAAACGCC |
| | | GGGTTCGTCT TCTACGATGA GAACGCCCAA CTTGTTCGAG TCAAGGTCCG |
| | | AGACTGCCTG GACCATCTCA AACTGAGATA CAAATATCAG GATATTCCTA |
| | | TTCCATAGCT AAAAAAAAGT ACGCAGCCCA AAGCGAAGAC GACCACCAAA |
| | | AGTTCCAAGT CGGGAGTAGC GAAAGCGGCG GAACTCCCAA AGACGACGAT |
| | | CAGCAGCAGT GGAGGCTTCC CAAAACCTCT TAACTCAGTA ATAAGAGTAG |
| | | AAGTTCCATG GCCAAAGAAA TCAAGAAGCA AGAAGAAGAA AGAGGATGAA |
| | | GAAGAGGTGT TGCTGAAGAC TATTCAGTAA GCCCAGCAAT AGTGAGTTAA |
| | | CAGGAAGCTT TGTTAACGTA CCACAAGCAT AAGCAGAGAA TGAAGACGAA |
| | | GACCAGCCTG CTCAGTTAGC GATAAAGAGG ACTGGGGCTG AAGACGATGA |
| | | CAGTTGTCCC TCATGCTCGG ACTGATGATG CCACCATTGG TGGAATTGAG |
| | | ATCGAGTTTA TTTCTGATTG ATTCCACCTT TCAATTATCA TCCATAATAT |
| | | ATATCCGATC AAGGAAAAGT CCATGCGGTA CTGCACCAAT GCAGTACTAC |
| | | ACCCAATGCA GTACTACACC AAATGCAGTA CTGTAGCCTA CTCCGAATTC |
| | | AGTACTACAC CAAATGCAGT AGTACCAAAA CTTTTTTCAT CTGTTGACTT |
| | | GGCCATCCAT TTTAATTTTT TTTTTTTAAA AAAGATATGA CCGGTACCAA |
| | | AGATTGAGGG TATGGTACCA AAACATGTGG CCTATTGTTA TTGGTGGAAG |
| | | ATTCAGGGTA TTGGGTACCC AAGCATGACC TGATCATGTG TTTTTCAAAA |
| | | TCAATGAAGG TCATTTCCGC GGATGATGCA TCATGTATGT GAAATTGCTT |
| | | TCTACCTTCC CCTCCAAGTC TTTTGCTAAT CTAGAGCTGT TTTCTTTATA |
| | | TTAAGGGATT TTTAATTAGG GATAGTTTCT TGTGCCTTCC TTCTCCTTTA |
| | | CATCGAAATT GAAGTTGAAG CCGAAGTCAA TTAAATTCAT AATTTTTATA |
| | | AGACATTATT GATATTATAA AAATTTTATT AAATATAAAA TAAATTTTTA |
| | | TATAAGTTCA AATATAATTT TTATAATACA AAATTTATTT TACTTTTGTG |
| | | TTAGTTATTT TTTTTTTTTT TAAGTTATAA GGTCTTTAAA TAAAATTAAT |
| | | CAAATAGATA TGTACACATT CAAATTCTAA TTATAGGGCT TAGAAAATAG |
| | | TTTTTGGTTT CTTAAAAAGT CGATCCAAGT AGATCCTACA TATATCACGG |
| | | GATATTCAAG ATCATATGAG TCGAAATTTC AAATTAGGTC CGGAACTAGA |
| | | GGCTTATTTG GGCAATGTCT TTCTTGTCTC TAGTTTTCAA GTATGGTTCC |
| | | TAAATAGACC TTTAAAGTTC TTGAGATAGA AAATTAGATA TAGTAGAAAA |
| | | TTATAGTTTA TAATTTTTTT TTTAATATAA AAAAGAAAAT AAGAGGAACA |
| | | AAATCGAAAA AGCATCTTAA ACAAAATTTA TGGAGCTGAA CCAACCTTGT |
| | | GTGCTTGTGT TTCTTCTCCC TTATTTTCCC TTTTATCAAA CTTTTCAAAA |
| | | TTCAAAAGTG GCTTTGAAAA CCTTGTTTTG CATAGAGAGC TAGCTGATTT |
| | | TAATCAGAGC CACAAGCAAA AATATCAAAT CCATTGCCTA ATTAATCATT |
| | | TGACTTACTA TCCGATTTGG GTTTTGGATT TAATGTGAAC AATTTTCAAA |
| | | CTCTAACTAC TTGAAACCCA ATTCTAGATT GGGCCTGGCC CAAGTTGAAG |
| | | CGGCCTTTTT AACCCATGCT GAAAGGTTGC TGCTTCAATT TGAATACCTC |
| | | GCGAGGGTAT TGTCTTTGAA AGAACAACTT TACTTTAGAT TTATTTATAT |
| | | ACATAGAGAG AGATATGAAA ATAGATTTAA AGTTAATAAA TTATTTTTAC |
| | | ATGTTTTTTG AAATTTGTTA TACATATTTT TTTATCTTCC ATATAAAAAT |
| | | TAAATAATTT TAAAATGTAT AAATGTCTAA CAAATTTGAA TTATATCTTC |
| | | TTTTTTTAAT TTGAATTATA TTTAATTTTT TATATATTTC TTATTGTAAA |
| | | TGGAACTTAA GAAAACATTT TACTTATCAT TTCTGTTCTT TTTATTTTCC |
| | | TTCAAATAAA ACAAATTTAC CATTTTTTTA CTTAGTATTT TCCTTGGAAT |
| | | CAAACATAGT TCAACTCTGT CTTAGAAAGA AAGAAAAATA AATGAAATAC |
| | | TAGATATGAT GATCTTCAAA GACTCATAAA TCGAATCTTA AGAAATAAGT |
| | | AAAATATTTG AGGATAAATA TCAAATTTCA ATTTAAAATA ATTTATCATA |
| | | GTTATGAGTT AAACTTGATC ATTATCCTTG AAATTATTTA AGGGGATATG |
| | | GTTGTTTCAT ATAGGATATC ATGATGTCCT TATTGAGAGT TTCCATGTTG |
| | | TAGCATGTTG AATAAAATAA TTGTAGGAAA CTTTGGATTA CTTCATTCTC |
| | | CGACCTTAGT TCATTAAGGT GCATGCAATC ATTAAGGTGA AGTAGTGGGA |
| | | TTTCGGGTTT TTGAGAATAT GGGGAAGAAG AGGAGAGGTT AGGGATTCAT |
| | | GGAATTTCAT GATTTTTGGA GAAGTGAAAG AAGTTAAGGT TTCAAGAAAT |
| | | TTTGAGAATG AGGAAGAAAA GGAAAGGTTA GATGATTCAT GGGATTTTTA |
| | | GAGAAAAAAC GAGATTAGGG CTTCTAGAGA TCTTGAGAAA AGAGGTAGAA |
| | | TTGGTTAAGA CTTTGTTGGA ATTTTGGGTT TGTGTCTTTC TCTTCTTTGA |
| | | TTTTTTTTTT TTTCACATGT GGGAGACTTA GCTGGCAATG TGGATGGCAT |
| | | TTTTTAAATT GAATTACACA CAAGCTAAAA GTATATGAGG AAGGATTTTA |
| | | GATCGAATCA TAAAAGTTCT GTTGTTCGTG TAGATATCTT CGAAGTCTGC |
| | | TTGA |
| 46 | Vitis | ATGGCGCTCT ATTCTCTCTT CAGATCCGCG CCTTCCGCCG CCGCCGCCGC |
| | vinifera | CGCCGCTACT GTGGTGGATT CTTCTCGTCC TTCTGATCGA TGCAAGGTGT |
| | GAPDH | CTTGCATTGG TTTCAATAGC AATGCGGACT CACTGAAGGT TCAGACCACC |
| | coding | ATATTTGGTA CTCCTTGTAT TGGAAATTCA TCTTCCTTAC AGAAATGCAG |
| | sequence | TGCCAGGAGT CTCCAGCCCA TCAAAGCCAC AGCCACAGAA TTGCCTCCTA |
| | | CAGTTCAAAA GTCATGGAGC AGTGGGCAGA CAAAGATTGG AATCAATGGT |
| | | TTTGGTCGAA TTGGAAGGTT GGTTTTACGA GTAGCAACCT CTAGGGATGA |
| | | TATTGATGTG GTAGCAGTCA ATGATCCATT TGTTGATGCC AAATACATGG |
| | | CTTACATGTT CAAGTATGAT TCTACTCATG GAGTTTTCAA GGGAACCATC |
| | | AAGGTATTGG ATGAGTCCAC TTTGGAAATC AATGGCAAGC AGATAAAAGT |
| | | TACAAGCAAA AGGGACCCAT CAGAAATCCC TTGGGGTGAT TATGGGATTG |
| | | ATTATGTTGT TGAATCTTCT GGAGTTTTCA CAACAACCGA GAAGGCTTCT |
| | | GCACACATAA AGGGTGGTGC CAAGAAAGTG GTAATTTCAG CTCCATCAGC |
| | | TGATGCACCG ATGTTTGTGG TAGGAGTAAA TGAGACTTCA TACAAGCCAA |
| | | GCATGAATAT TGTTTCTAAT GCAAGCTGTA CCACCAATTG CCTTGCTCCT |
| | | CTTGCCAAGG TTGTTCATGA GGAATTTGGT ATCGTTGAGG GTTTAATGAC |
| | | AACTGTGCAT GCCACTACAG CCACACAGAA GACCGTGGAT GGGCCTTCAA |
| | | TGAAGGATTG GCGAGGGGGC CGAGGTGCTT CACAAAATAT CATCCCAAGC |
| | | TCCACTGGTG CTGCAAAGGC TGTTGGAAAA GTCCTTCCAG AACTCAACGG |
| | | AAAGCTCACT GGAATGGCCT TCCGCGTCCC AACACCTAAT GTCTCTGTTG |
| | | TGGACTTAAC TTGTCGACTT AAGAAGAGTG CTACATATGA AGATGTCAAA |
| | | GCAGCCGTAA AGTATGCCTC AGAGGGGCCA TTGAAGGGTA TTCTTGGATA |
| | | CACGGATGAG GATGTTGTCT CCAATGATTT TGTTGGTGAC TCAAGGTCAA |
| | | GTATATTTGA TGCAAAGGCC GGGATAGGGC TGAGTGCTTC CTTCATGAAG |
| | | CTCGTTGCAT GGTATGACAA CGAATGGGGA TACAGCAACC GAGTGCTGGA |
| | | CCTCATAGAG CACATGGCAT TGGTGGCGGC CAGCAAATGA |
| 47 | Vitis | MALYSLFRSA PSAAAAAAAT VVDSSRPSDR CKVSCIGFNS NADSLKVQTT |
| | vinifera | IFGTPCIGNS SSLQKCSARS LQPIKATATE LPPTVQKSWS SGQTKIGING |
| | GAPDH | FGRIGRLVLR VATSRDDIDV VAVNDPFVDA KYMAYMFKYD STHGVFKGTI |
| | amino acid | KVLDESTLEI NGKQIKVTSK RDPSEIPWGD YGIDYVVESS GVFTTTEKAS |
| | sequence | AHIKGGAKKV VISAPSADAP MFVVGVNETS YKPSMNIVSN ASCTTNCLAP |
| | | LAKVVHEEFG IVEGLMTTVH ATTATQKTVD GPSMKDWRGG RGASQNIIPS |
| | | STGAAKAVGK VLPELNGKLT GMAFRVPTPN VSVVDLTCRL KKSATYEDVK |
| | | AAVKYASEGP LKGILGYTDE DVVSNDFVGD SRSSIFDAKA GIGLSASFMK |
| | | LVAWYDNEWG YSNRVLDLIE HMALVAASK |
| 48 | Vitis | ATGGGTGGAA ACTATGGCAA CCGAGAGATC AGAGTGCCTT TTGCACATTT |
| | vinifera | TCTTATGGGA AGGTCTGGTA GATTTAACTT AGATGTCCCA TATTCATCTC |
| | ACTIN2 | GTCTTCAACA GCAACAACAA CATGCTTCAT CAATTGGAAG CGGTGGACCA |
| | coding | CCTTCCATAG GTCCATTACT GCTGCCACCG CAACTGCCTC AATCTCTTTT |
| | sequence | TCCTCTTCAC ATGGGTTCAG CCGCTCCTCG TCCCGCCGTG ATCATTAACT |
| | | ACGGCACCAG GTATATTAAA ATGGGTTTTT CCGGTAACGT GGAGCCGTGC |
| | | TTCATTGCCC CGACAGTTGT TGCTGTCAAC GAGTCGTTTT TGAATCAAAA |
| | | TCGGAGCACC ACCAACCCAA GCTATCCGAT TGTTCACGGC CAGGTTTATA |
| | | ATTGGGATGT CATGGAGAGA TTTTGGCAGC AGGGTATATT TAATTATTTG |
| | | AGATGTGATC AGGAGGATCA CTACTTTCTT TTGGCGGGAA GTCCACTCAC |
| | | TGCCCCGGAG AGCCGTGAAT ACACTGGTGA AATTATGTTT GAAACCTTCA |
| | | AATGTTCCTG GTCTTTATAT TGCAGTGCAG CCGGTACTCG CCCATGTGAC |
| | | AGGATACACT ACATCCAAGT GGCAGTAGAT GACAAGGATG GTGCAATTTC |
| | | TGGGGAGATA AGAAGCAATG GCCTTAAAGC CTACGTTCAT CTATATCTTG |
| | | CTGCACTCAA TGGTGATTGG AAAAGTGCAA AAGCATTCCT GGAGTCAAAT |
| | | CCACAAACAG TGAGAACGAG GATCACAAGG CTCTTAGAAA CTGCTCTTCA |
| | | TATTGCTGCT ATGGCAGGAT TTGCATCTGC AAGTGATAGA GGTATCAGGA |
| | | GAAGCAGATA A |
| 49 | Vitis | MGGNYGNREI RVPFAHFLMG RSGRFNLDVP YSSRLQQQQQ HASSIGSGGP |
| | vinifera | PSIGPLLLPP QLPQSLFPLH MGSAAPRPAV IINYGTRYIK MGFSGNVEPC |
| | ACTIN2 | FIAPTVVAVN ESFLNQNRST TNPSYPIVHG QVYNWDVMER FWQQGIFNYL |
| | amino acid | RCDQEDHYFL LAGSPLTAPE SREYTGEIMF ETFKCSWSLY CSAAGTRPCD |
| | sequence | RIHYIQVAVD DKDGAISGEI RSNGLKAYVH LYLAALNGDW KSAKAFLESN |
| | | PQTVRTRITR LLETALHIAA MAGFASASDR GIRRSR |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A grape plant cell comprising one or more loss-of-function mutations of a nucleic acid sequence encoding a polyphenol oxidase (PPO) gene selected from one or more of PPO1, PPO2, PPO3, PPO4, and PPO5, wherein the grape plant cell has reduced polyphenol oxidase gene expression or activity compared to a wild-type grape plant cell.

2. The grape plant cell of claim 1, wherein the one or more loss-of-function mutations are in at least one chromosomal allele of the nucleic acid sequence.

3. The grape plant cell of claim 1, wherein the one or more loss-of-function mutations are in both chromosomal alleles of the nucleic acid sequence.

4. The grape plant cell of any one of claims 1-3, wherein the one or more loss-of-function mutations are in the PPO1 nucleotide sequence of SEQ ID NO:2, or a nucleotide sequence that has at least 95% sequence identity to SEQ ID NO:2.

5. The grape plant cell of any one of claims 1-4, wherein the one or more loss-of-function mutations are in the PPO2 nucleotide sequence of SEQ ID NO:5, or a nucleotide sequence that has at least 95% sequence identity to SEQ ID NO: 5.

6. The grape plant cell of any one of claims 1-5, wherein the one or more loss-of-function mutation are in the PPO3 nucleotide sequence of SEQ ID NO: 8, or a nucleotide sequence that has at least 95% sequence identity to the nucleotide sequence of SEQ ID NO:8.

7. The grape plant cell of any one of claims 1-6, wherein the one or more loss-of-function mutations are in the PPO4 nucleotide sequence of SEQ ID NO:11, or a nucleotide sequence that has at least 95% sequence identity to the nucleotide sequence of SEQ ID NO:11.

8. The grape plant cell of any one of claims 1-7, wherein the one or more loss-of-function mutations are in the PPO5 nucleotide sequence of SEQ ID NO:14, or a nucleotide sequence that has at least 95% sequence identity to the nucleotide sequence of SEQ ID NO:14.

9. The grape plant cell of claim 1 comprising a loss-of-function mutation in both chromosomal alleles of the PPO2 gene and a loss-of-function mutation in both chromosomal alleles of one or more of the PPO1, PPO3, PPO4, and PPO5 genes.

10. The grape plant cell of claim 1 comprising a loss-of-function mutation in both chromosomal alleles of the PPO2 gene and a loss-of-function mutation in both chromosomal alleles of the PPO1 and PPO3 genes.

11. The grape plant cell of claim 1 comprising a loss-of-function mutation in both chromosomal alleles of the PPO2 gene and a loss-of-function mutation in both chromosomal alleles of the PPO1, PPO3, and PPO5 genes.

12. The grape plant cell of any one of claims 1, 4, or 9-11, wherein the one or more loss-of-function mutations in PPO1 are selected from SEQ ID NOs:68-70.

13. The grape plant cell of any one of claims 1, 5, or 9-11, wherein the one or more loss-of-function mutations in PPO2 are selected from SEQ ID NOs:71-73.

14. The grape plant cell of any one of claims 1, 6, or 9-11, wherein the one or more loss-of-function mutations in PPO3 are selected from SEQ ID NOs:74-76.

15. The grape plant cell of any one of claims 1, 8, or 9-11, wherein the one or more loss-of-function mutations in PPO5 are selected from SEQ ID NOs:77-79.

16. The grape plant cell of any one of claims 1-15, wherein the grape plant cell is a protoplast.

17. The grape plant cell of any one of claims 1-16, wherein the loss-of-function mutation(s) encodes a knockout mutation.

18. The grape plant cell of any one of claims 1-17, wherein the grape plant cell is free of exogenous DNA.

19. The grape plant cell of any one of claims 1-18, wherein the polyphenol oxidase gene activity is reduced by at least 10% compared to polyphenol oxidase gene expression or activity of a wild-type grape plant cell.

20. The grape plant cell of any one of claims 1-19, wherein the polyphenol oxidase activity is reduced by at least 20% compared to polyphenol oxidase gene expression or activity of a wild-type grape plant cell.

21. A grape plant comprising the grape plant cell of any one of claims 1-20.

22. A grape fruit comprising the grape plant cell of any one of claims 1-20.

23. A grape plant, plant part, seed, or fruit propagated from the grape cell of any one of claims 1-20, the grape plant of claim 21, or the grape fruit of claim 22, wherein the grape plant, plant part, seed, or fruit comprises a loss-of-function mutation in a polyphenol oxidase (PPO) gene.

24. A beverage made from the grape fruit of claim 22 or claim 23.

25. A food product made from the grape fruit of claim 22 or claim 23.

26. The grape plant of claim 21, grape plant part or seed of claim 22, grape fruit of claim 23, beverage of claim 24, or food product of claim 25 comprise a characteristic selected from the group consisting of reduced browning, increased shelf life, and increased polyphenolics compared to a wild-type grape plant, plant part, seed, fruit, beverage, or food product.

27. The grape plant cell of any one of claims 1-20, wherein the grape is of the variety Arbane, Chardonnay, Chenin Blanc, Gewürztraminer, Grenache, Grenache Blanc, Gruner Veltliner, Gamay, Marsanne, Muscat, Pinot Blanc, Pinot Gris, Reisling, Roussanne, Sauvignon Blanc, Sémillion, Viognier, Pinot Noir, Tempranillo, Sangiovese, Merlot, Zinfandel, Cabernet Sauvignon, Nebbiolo, Syrah, Petit Sirah, Malbec, Shiraz, Garnacha, Páis Grenache, Mourvédre, Muscat, Picpoul, Petit Meslier, Pixie, Terret, Counoise, Muscardin, Rioja, Rose, Barbera, Cabernet Franc, Carignane, Charbono, Tinto Madeira, Vaccarēse, Picordin, Cinsault, Clairette, Roussanne, Bourboulenc, Dolcetto, and Barbera, or a hybrid thereof.

28. A method of making a grape plant with reduced polyphenol oxidase (PPO) gene expression or activity, said method comprising:
introducing one or more loss-of-function mutations into a nucleic acid sequence encoding a polyphenol oxidase (PPO) gene selected from one or more of PPO1, PPO2, PPO3, PPO4, and PPO5 of a grape plant cell, wherein the grape plant cell has reduced polyphenol oxidase gene expression or activity compared to a wild-type grape plant cell; and
regenerating a plant from the grape plant cell.

29. The method of claim 28, wherein the grape plant cell is a protoplast.

30. The method of claim 28 or claim 29, wherein said introducing comprises:
transfecting the plant cell with at least one ribonucleoprotein (RNP) complex comprising a guide RNA and a genome editing nuclease; and
editing the plant cell's genome to induce the one or more loss-of-function mutations.

31. The method of claim 30, comprising two or more different RNP complexes.

32. The method of claim 30, wherein the guide RNA comprises one or more of SEQ ID NOs: 17-24.

33. The method of any one of claims 28-32, wherein the one or more loss-of-function mutations are in at least one chromosomal allele of the nucleic acid sequence.

34. The method of any one of claims 28-33, one or more loss-of-function mutations are in both chromosomal alleles of the nucleic acid sequence.

35. The method of any one of claims 28-34, wherein the one or more loss-of-function mutations are in the nucleic acid sequence encoding the PPO2 gene.

36. The method of any one of claims 28-35, wherein said introducing is carried out with respect to the PPO2 gene and one or more of the PPO1, PPO3, PPO4, and PPO5 genes.

37. The method of any one of claims 28-36, wherein the polyphenol oxidase gene expression or activity is reduced by at least 10% compared to the polyphenol oxidase gene expression or activity of a wild-type grape plant.

38. The method of any one of claims 28-37, wherein the grape plant is of the variety Arbane, Chardonnay, Chenin Blanc, Gewürztraminer, Grenache, Grenache Blanc, Gruner Veltliner, Gamay, Marsanne, Muscat, Pinot Blanc, Pinot Gris, Reisling, Roussanne, Sauvignon Blanc, Sémillion, Viognier, Pinot Noir, Tempranillo, Sangiovese, Merlot, Zinfandel, Cabernet Sauvignon, Nebbiolo, Syrah, Petit Sirah, Malbec, Shiraz, Garnacha, Páis Grenache, Mourvédre, Muscat, Picpoul, Petit Meslier, Pixie, Terret, Counoise, Muscardin, Rioja, Rose, Barbera, Cabernet Franc, Carignane, Charbono, Tinto Madeira, Vaccarēse, Picordin, Cinsault, Clairette, Roussanne, Bourboulenc, Dolcetto, and Barbera, or a hybrid thereof.
